(19) United States
(12) Patent Application Publication
(10) Pub. No.: US 2003/0044864 A1
(43) Pub. Date: Mar. 6, 2003

(54) Cellular engineering, protein expression profiling, differential labeling of peptides, and novel reagents therefor

(75) Inventors: Jay M. Short, Rancho Santa Fe, CA (US); Martin Latterich, San Diego, CA (US); Jing Wei, San Diego, CA (US); Michael Levin, San Diego, CA (US)

Correspondence Address: FISH & RICHARDSON, PC, 4350 LA JOLLA VILLAGE DRIVE, SUITE 500, SAN DIEGO, CA, 92122, US

(73) Assignee: Diversa Corporation, San Diego, CA (US)

(21) Appl. No.: 10/202,399

(22) Filed: Jul. 22, 2002

Related U.S. Application Data
(60) Provisional application No. 60/307,064, filed on Jul. 20, 2001.
(60) Provisional application No. 60/337,526, filed on Nov. 9, 2001.
(60) Provisional application No. 60/326,654, filed on Oct. 1, 2001.

(51) Int. Cl. G01N 33/574; G06F 19/00; G01N 33/48; G01N 33/50

(52) U.S. Cl. 435/7.23; 702/19

(57) ABSTRACT

The invention provides cellular transformation, directed evolution, and screening methods for creating novel transgenic organisms having desirable properties. Thus in one aspect, this invention relates to a method of generating a transgenic organism, such as a microbe or a plant, having a plurality of traits that are differentially activatable. Also, a method of retooling genes and gene pathways by the introduction of regulatory sequences, such as promoters, that are operable in an intended host, thus conferring operability to a novel gene pathway when it is introduced into an intended host. For example a novel man-made gene pathway, generated based on microbially-derived progenitor templates, that is operable in a plant cell. Furthermore, a method of generating novel host organisms having increased expression of desirable traits, recombinant genes, and gene products. Additionally, the invention provides novel methods for determining polypeptide profiles, and protein expression variations, which methods are applicable to all sample types disclosed herein. The present invention provides methods of simultaneously identifying and quantifying individual proteins in complex protein mixtures. Protein expression levels can be globally quantified.

### TECHNICAL FIELD

This invention relates to proteomics and mass spectrometry technology. In particular, the invention provides novel methods for determining polypeptide profiles and protein expression variations, as with proteome analyses. The present invention provides methods of simultaneously identifying and quantifying individual proteins in complex protein mixtures by selective differential labeling of amino acid residues followed by chromatographic and mass spectrographic analysis.

### BACKGROUND

The predisposition for diagnosis and treatment of a variety of diseases and disorders may often be accomplished through identification and quantitative measurement of polypeptide expression variations between different cell types and cell states. Biochemical pathways and metabolic networks can also be analyzed by globally and quantitatively measuring protein expression in various cell types and biological states (see, e.g., Ideker (2001) Science 292:929-934).

State-of-the-art techniques such as liquid-chromatography-electospray-ionization tandem mass spectrometry have, in conjunction with database-searching computer algorithms, revolutionized the analysis of biochemical species from complex biological mixtures. With these techniques, it is now possible to perform high-throughput protein identification at picomolar to subpicomolar levels from complex mixtures of biological molecules (see, e.g., Dongre (1997) Trends Biotechnol. 15:418-425).

One such method is based on a class of chemical reagents termed isotope-coded affinity tags (ICATs) and tandem mass spectrometry. The method labels multiple cysteinyl residues and uses stable isotope dilution techniques. For example, Gygi (1999) Nat. Biotechnol. 10:994-999, compared protein expression in a yeast using ethanol or galactose as a carbon source. The measured differences in protein expression correlated with known yeast metabolic function under glucose-repressed conditions.

In another technique, two different protein mixtures for quantitative comparison are digested to peptide mixtures, the peptides mixtures are separately methylated using either d0- or d3-methanol, the mixtures of methylated peptide combined and subjected to microcapillary HPLC-MS/MS (see, e.g., Goodlett, David R., et al., (2000) “Differential Isotopic Labeling of Peptides for Global Quantification of Proteins and de novo Sequence Derivation,” 49th ASMS). Parent proteins of methylated peptides are identified by correlative database searching of fragment ion spectra using a computer program assisted paradigms or automated de novo sequencing that compares all tandem mass spectra of d0- and d3-methylated peptide ion pairs. In Goodlett (2000) supra, ratios of proteins in two different mixtures were calculated for d0- to d3-methylated peptide pairs. However, there are several limitations to this approach, including: use of differential labeling reagents, which relied on stable isotopes, which are expensive, and not flexible to differential labeling of more than two mixtures of peptides; labeling methods limited only to methylation of carboxy-termini; protein expression profiling limited to duplex comparison; one dimensional capillary HPLC chromatography was employed to separate peptides, which doesn't has enough capacity and resolving power for complex mixtures of peptides.

### Screening and Selection

Overview of Screening and Selection

Screening is, in general, a two-step process in which one first determines which cells do and do not express a screening marker and then physically separates the cells having the desired property. Screening markers include, for example, luciferase, beta-galactosidase, and green fluorescent protein. Screening can also be done by observing a cell holistically including but not limited to utilizing methods pertaining to genomics, RNA profiling, proteomics, metabolomics, and lipidomics as well as observing such aspects of growth as colony size, halo formation, etc. Additionally, screening for production of a desired compound, such as a therapeutic drug or “designer chemical” can be accomplished by observing binding of cell products to a receptor or ligand, such as on a solid support or on a column. Such screening can additionally be accomplished by binding to antibodies, as in an ELISA. In some instances the screening process can be automated so as to allow screening of suitable numbers of colonies or cells. Some examples of automated screening devices include fluorescence activated cell sorting (FACS), especially in conjunction with cells immobilized in agarose (see Powell et. al. Bio/Technology 8:333-337 (1990); Weaver et. al. Methods 2:234-247 (1991)), automated ELISA assays, scintillation proximity assays (Hart, H. E. et al., Molecular Immunol. 16:265-267 (1979)) and the formation of fluorescent, colored or UV absorbing compounds on agar plates or in microtiter wells (Krawiec, S., Devel. Indust. Microbiology 31:103-114 (1990)).

Selection is a form of screening in which identification and physical separation are achieved simultaneously, for example, by expression of a selectable marker, which, in some genetic circumstances, allows cells expressing the marker to survive while other cells die (or vice versa). Selectable markers can include, for example, drug, toxin resistance, or nutrient synthesis genes. Selection is also done by such techniques as growth on a toxic substrate to select for hosts having the ability to detoxify a substrate, growth on a new nutrient source to select for hosts having the ability to utilize that nutrient source, competitive growth in culture based on ability to utilize a nutrient source, etc.

In particular, uncloned but differentially expressed proteins (e.g., those induced in response to new compounds, such as biodegradable pollutants in the medium) can be screened by differential display (Appleyard et al. Mol. Gen. Gent. 247:338-342 (1995)). Hopwood (Phil Trans R. Soc. Lond B 324:549-562) provides a review of screens for antibiotic production. Omura (Microbio. Rev. 50:259-279 (1986) and Nisbet (Ann Rev. Med. Chem. 21:149-157 (1986)) disclose screens for antimicrobial agents, including supersensitive bacteria, detection of beta-lactamase and D,D-carboxypeptidase inhibition, beta-lactamase induction, chromogenic substrates and monoclonal antibody screens.

Antibiotic targets can also be used as screening targets in high throughput screening. Antifungals are typically screened by inhibition of fungal growth. Pharmacological agents can be identified as enzyme inhibitors using plates containing the enzyme and a chromogenic substrate, or by automated receptor assays. Hydrolytic enzymes (e.g., proteases, amylases) can be screened by including the substrate in an agar plate and scoring for a hydrolytic clear zone or by using a colorimetric indicator (Steele et al. Ann. Rev. Microbiol. 45:89-106 (1991)). This can be coupled with the use of stains to detect the effects of enzyme action (such as congo red to detect the extent of degradation of celluloses and hemicelluloses).

Tagged substrates can also be used. For example, lipases and esterases can be screened using different lengths of fatty acids linked to umbelliferyl. The action of lipases or esterases removes this tag from the fatty acid, resulting in a quenching or enhancement of umbelliferyl fluorescence. These enzymes can be screened in microtiter plates by a robotic device.

High-throughput Cellular Screening: Utilizing Various Types of “Omics”

Functional genomics seeks to discover gene function once nucleotide sequence information is available. Proteomics (the study of protein properties such as expression, post-translational modifications, interactions, etc.) and metabolomics (analysis of metabolite pools) are fast-emerging fields complementing functional genomics, that provide a global, integrated view of cellular processes. The variety of techniques and methods used in this effort include the use of bioinformatics, gene-array chips, mRNA differential display, disease models, protein discovery and expression, and target validation. The ultimate goal of many of these efforts has been to develop high-throughput screens for genes of unknown function. For review see Greenbaum D. et al. Genome Res, 11(9):1463-8 (2001).

Genomics

Genomics can refer to various investigative techniques that are broad in scope but often refers to measuring gene expression for multitudes of genes simultaneously. For a review see Lockhart, D. J. and Winzeler, E. A. 2000. Genomics, gene expression and DNA arrays. Nature, 405(6788):827-36.

Biological Chips

General Considerations

In some systems, an oligonucleotide probe is tethered, i.e., by covalent attachment, to a solid support, and arrays of oligonucleotide probes immobilized on solid supports have been used to detect specific nucleic acid sequences in a target nucleic acid. See, e.g., PCT patent publication Nos. WO 89/10977 and 89/11548. Others have proposed the use of large numbers of oligonucleotide probes to provide the complete nucleic acid sequence of a target nucleic acid but failed to provide an enabling method for using arrays of immobilized probes for this purpose. See U.S. Pat. Nos. 5,202,231 and 5,002,867 and PCT patent publication No. WO 93/17126. See U.S. Pat. No. 5,143,854 and PCT patent publication Nos. WO 90/15070 and 92/10092. Microfabricated arrays of large numbers of oligonucleotide probes, called “DNA chips” offer great promise for a wide variety of applications. New methods and reagents are required to realize this promise.

Informatics

Informatics is the study and application of computer and statistical techniques to the management of information. In genome projects, bioinformatics includes the development of methods to search databases quickly, to analyze nucleic acid sequence information, and to predict protein sequence, structure and function from DNA sequence data. Increasingly, molecular biology is shifting from the laboratory bench to the computer desktop. Today's researchers require advanced quantitative analyses, database comparisons, and computational algorithms to explore the relationships between sequence and phenotype. Thus, by all accounts, researchers can not and will not be able to avoid using computer resources to explore gene expression, gene sequencing and molecular structure.

One use of bioinformatics involves studying an organism's genome to determine the sequence and placement of its genes and their relationship to other sequences and genes within the genome or to genes in other organisms. Another use of bioinformatics involves studying genes differentially or commonly expressed in different tissues or cell lines (e.g. normal and cancerous tissue). Such information is of significant interest in biomedical and pharmaceutical research, for instance to assist in the evaluation of drug efficacy and resistance.

The sequence tag method involves generation of a large number (e.g., thousands) of Expressed Sequence Tags (“ESTs”) from cDNA libraries (each produced from a different tissue or sample). ESTs are partial transcript sequences that may cover different parts of the cDNA(s) of a gene, depending on cloning and sequencing strategy. Each EST includes about 50 to 300 nucleotides. If it is assumed that the number of tags is proportional to the abundance of transcripts in the tissue or cell type used to make the cDNA library, then any variation in the relative frequency of those tags, stored in computer databases, can be used to detect the differential abundance and potentially the expression of the corresponding genes.

To make genomic and EST information manipulation easy to perform and understand, sophisticated computer database systems have been developed. In one database system, developed by Incyte Pharmaceuticals, Inc. of Palo Alto, Calif., genomic sequence data and the abundance levels of mRNA species represented in a given sample is electronically recorded and annotated with information available from public sequence databases such as GenBank. Examples of such databases include GenBank (NCBI) and TIGR. The resulting information is stored in a relational database that may be employed to determine relationships between sequences and genes within and among genomes and establish a cDNA profile for a given tissue and to evaluate changes in gene expression caused by disease progression, pharmacological treatment, aging, etc.

In one database system, developed by Incyte Pharmaceuticals, Inc. of Palo Alto, Calif., abundance levels of mRNA species represented in a given sample are electronically recorded and annotated with information available from public sequence databases such as GenBank. The resulting information is stored in a relational database that may be employed to establish a cDNA profile for a given tissue and to evaluate changes in gene expression caused by disease progression, pharmacological treatment, aging, etc.

Genetic information for a number of organisms has been catalogued in computer databases. Genetic databases for organisms such as Escherichia coli, Haemophilus influenzae, Mycoplasma genitalium, and Mycoplasma pneumoniae, among others, are publicly available. At present, however, complete sequence data is available for relatively few species, and the ability to manipulate sequence data within and between species and databases is limited.

While genetic data processing and relational database systems such as those developed by Incyte Pharmaceuticals, Inc. provide great power and flexibility in analyzing genetic information and gene expression information, this area of technology is still in its infancy and further improvements in genetic data processing and relational database systems and their content will help accelerate biological research for numerous applications.

In genome projects, bioinformatics includes the development of methods to search databases quickly, to analyze nucleic acid sequence information, and to predict protein sequence and structure from DNA sequence data. Increasingly, molecular biology is shifting from the laboratory bench to the computer desktop. Advanced quantitative analyses, database comparisons, and computational algorithms are needed to explore the relationships between sequence and phenotype.

The predisposition for or diagnosis and treatment of a variety of diseases and disorders may often be accomplished through identification and quantitative measurement of polypeptide expression variations between different cell types and cell states. Biochemical pathways and metabolic networks can also be analyzed by globally and quantitatively measuring protein expression in various cell types and biological states (see, e.g., Ideker (2001) Science 292:929-934).

State-of-the-art techniques such as liquid-chromatography-electrospray-ionization tandem mass spectrometry have, in conjunction with database-searching computer algorithms, revolutionized the analysis of biochemical species from complex biological mixtures. With these techniques, it is now possible to perform high-throughput protein identification at picomolar to subpicomolar levels from complex mixtures of biological molecules (see, e.g., Dongre (1997) Trends Biotechnol. 15:418-425).

One such method is based on a class of chemical reagents termed isotope-coded affinity tags (ICATs) and tandem mass spectrometry. The method labels multiple cysteinyl residues and uses stable isotope dilution techniques. For example, Gygi (1999) Nat. Biotechnol. 10:994-999, compared protein expression in a yeast using ethanol or galactose as a carbon source. The measured differences in protein expression correlated with known yeast metabolic function under glucose-repressed conditions.

In another technique, two different protein mixtures for quantitative comparison are digested to peptide mixtures, the peptides mixtures are separately methylated using either d0- or d3-methanol, the mixtures of methylated peptide combined and subjected to microcapillary HPLC-MS/MS (see, e.g., Goodlett, David R., et al., (2000) “Differential Isotopic Labeling of Peptides for Global Quantification of Proteins and de novo Sequence Derivation,” 49th ASMS). Parent proteins of methylated peptides are identified by correlative database searching of fragment ion spectra using a computer program assisted paradigms or automated de novo sequencing that compares all tandem mass spectra of d0- and d3-methylated peptide ion pairs. In Goodlett (2000) supra, ratios of proteins in two different mixtures were calculated for d0- to d3-methylated peptide pairs. However, there are several limitations to this approach, including: use of differential labeling reagents, which relied on stable isotopes, which are expensive, and not flexible to differential labeling of more than two mixtures of peptides; labeling methods limited only to methylation of carboxy-termini; protein expression profiling limited to duplex comparison; one dimensional capillary HPLC chromatography was employed to separate peptides, which doesn't has enough capacity and resolving power for complex mixtures of peptides.

### SUMMARY

The invention provides methods for cellular screening, including cellular screening in genomics, e.g., as in high throughput genomics. “High throughput genomics” refers to application of genomic or genetic data or analysis techniques that use microarrays or other genomic technologies to rapidly identify large numbers of genes or proteins, or distinguish their structure, expression or function from normal or abnormal cells or tissues. In the methods of the invention, an observer can be a person viewing a slide with a microscope or an observer who views digital images. Alternatively, an observer can be a computer-based image analysis system, which automatically observes, analyses and quantitates biological arrayed samples with or without user interaction.

The present invention provides for the use of arrays of oligonucleotide probes immobilized in microfabricated patterns on silica chips for analyzing molecular interactions of biological interest.

The invention provides several strategies employing immobilized arrays of probes for comparing a reference sequence of known sequence with a target sequence showing substantial similarity with the reference sequence, but differing in the presence of, e.g., mutations. In one aspect, the invention provides a tiling strategy employing an array of immobilized oligonucleotide probes comprising at least two sets of probes. A first probe set comprises a plurality of probes, each probe comprising a segment of at least three nucleotides exactly complementary to a subsequence of the reference sequence, the segment including at least one interrogation position complementary to a corresponding nucleotide in the reference sequence. A second probe set comprises a corresponding probe for each probe in the first probe set, the corresponding probe in the second probe set being identical to a sequence comprising the corresponding probe from the first probe set or a subsequence of at least three nucleotides thereof that includes the at least one interrogation position, except that the at least one interrogation position is occupied by a different nucleotide in each of the two corresponding probes from the first and second probe sets. The probes in the first probe set have at least two interrogation positions corresponding to two contiguous nucleotides in the reference sequence. One interrogation position corresponds to one of the contiguous nucleotides, and the other interrogation position to the other.

In another aspect, the invention provides a tiling strategy employing an array comprising four probe sets. A first probe set comprises a plurality of probes, each probe comprising a segment of at least three nucleotides exactly complementary to a subsequence of the reference sequence, the segment including at least one interrogation position complementary to a corresponding nucleotide in the reference sequence. Second, third and fourth probe sets each comprise a corresponding probe for each probe in the first probe set.

The probes in the second, third and fourth probe sets are identical to a sequence comprising the corresponding probe from the first probe set or a subsequence of at least three nucleotides thereof that includes the at least one interrogation position, except that the at least one interrogation position is occupied by a different nucleotide in each of the four corresponding probes from the four probe sets. The first probe can have at least 100 interrogation positions corresponding to 100 contiguous nucleotides in the reference sequence. The first probe set can have an interrogation position corresponding to every nucleotide in the reference sequence. The segment of complementarity within the probe set is usually about 9 to 21 nucleotides. Although probes may contain leading or trailing sequences in addition to the 9-21 sequences, many probes consist exclusively of a 9-21 segment of complementarity.

In another aspect, the invention provides immobilized arrays of probes tiled for multiple reference sequences. one such array comprises at least one pair of first and second probe groups, each group comprising first and second sets of probes as defined in the first aspect. Each probe in the first probe set from the first group is exactly complementary to a subsequence of a first reference sequence, and each probe in the first probe set from the second group is exactly complementary to a subsequence of a second reference sequence.

Thus, the first group of probes are tiled with respect to a first reference sequence and the second group of probes with respect to a second reference sequence. Each group of probes can also include third and fourth sets of probes as defined in the second aspect. In some arrays of this type, the second reference sequence is a mutated form of the first reference sequence.

In another aspect, the invention provides arrays for block tiling. Block tiling is a species of the general tiling strategies described above. The usual unit of a block tiling array is a group of probes comprising a wildtype probe, a first set of three mutant probes and a second set of three mutant probes. The wildtype probe comprises a segment of at least three nucleotides exactly complementary to a subsequence of a reference sequence. The segment has at least first and second interrogation positions corresponding to first and second nucleotides in the reference sequence. The probes in the first set of three mutant probes are each identical to a sequence comprising the wildtype probe or a subsequence of at least three nucleotides thereof including the first and second interrogation positions, except in the first interrogation position, which is occupied by a different nucleotide in each of the three mutant probes and the wildtype probe. The probes in the second set of three mutant probes are each identical to a sequence comprising the wildtype probes or a subsequence of at least three nucleotides thereof including the first and second interrogation positions, except in the second interrogation position, which is occupied by a different nucleotide in each of the three mutant probes and the wildtype probe.

In another aspect, the invention provides methods of comparing a target sequence with a reference sequence using arrays of immobilized pooled probes. The arrays employed in these methods represent a further species of the general tiling arrays noted above. In these methods, variants of a reference sequence differing from the reference sequence in at least one nucleotide are identified and each is assigned a designation. An array of pooled probes is provided, with each pool occupying a separate cell of the array. Each pool comprises a probe comprising a segment exactly complementary to each variant sequence assigned a particular designation.

The array is then contacted with a target sequence comprising a variant of the reference sequence. The relative hybridization intensities of the pools in the array to the target sequence are determined. The identity of the target sequence is deduced from the pattern of hybridization intensities. Often, each variant is assigned a designation having at least one digit and at least one value for the digit. In this case, each pool comprises a probe comprising a segment exactly complementary to each variant sequence assigned a particular value in a particular digit. When variants are assigned successive numbers in a numbering system of base m having n digits, n×(m−1) pooled probes are used are used to assign each variant a designation.

In another aspect, the invention provides a pooled probe for trellis tiling, a further species of the general tiling strategy. In trellis tiling, the identity of a nucleotide in a target sequence is determined from a comparison of hybridization intensities of three pooled trellis probes. A pooled trellis probe comprises a segment exactly complementary to a subsequence of a reference sequence except at a first interrogation position occupied by a pooled nucleotide N, a second interrogation position occupied by a pooled nucleotide selected from the group of three consisting of (1) M or K, (2) R or Y and (3) S or W, and a third interrogation position occupied by a second pooled nucleotide selected from the group. The pooled nucleotide occupying the second interrogation position comprises a nucleotide complementary to a corresponding nucleotide from the reference sequence when the second pooled probe and reference sequence are maximally aligned, and the pooled nucleotide occupying the third interrogation position comprises a nucleotide complementary to a corresponding nucleotide from the reference sequence when the third pooled probe and the reference sequence are maximally aligned. Standard IUPAC nomenclature is used for describing pooled nucleotides.

In trellis tiling, an array comprises at least first, second and third cells, respectively occupied by first, second and third pooled probes, each according to the generic description above. However, the segment of complementarity, location of interrogation positions, and selection of pooled nucleotide at each interrogation position may or may not differ between the three pooled probes subject to the following constraint. One of the three interrogation positions in each of the three pooled probes must align with the same corresponding nucleotide in the reference sequence. This interrogation position must be occupied by a N in one of the pooled probes, and a different pooled nucleotide in each of the other two pooled probes.

In another aspect, the invention provides arrays for bridge tiling. Bridge tiling is a species of the general tiling strategies noted above, in which probes from the first probe set contain more than one segment of complementarity. In bridge tiling, a nucleotide in a reference sequence is usually determined from a comparison of four probes. A first probe comprises at least first and second segments, each of at least three nucleotides and each exactly complementary to first and second subsequences of a reference sequences. The segments including at least one interrogation position corresponding to a nucleotide in the reference sequence. Either (1) the first and second subsequences are noncontiguous in the reference sequence, or (2) the first and second subsequences are contiguous and the first and second segments are inverted relative to the first and second subsequences.

The arrays of the invention can further comprise second, third and fourth probes, which are identical to a sequence comprising the first probe or a subsequence thereof comprising at least three nucleotides from each of the first and second segments, except in the at least one interrogation position, which differs in each of the probes. In a species of bridge tiling, referred to as deletion tiling, the first and second subsequences are separated by one or two nucleotides in the reference sequence.

In another aspect, the invention provides arrays of probes for multiplex tiling. Multiplex tiling is a strategy, in which the identity of two nucleotides in a target sequence is determined from a comparison of the hybridization intensities of four probes, each having two interrogation positions. Each of the probes comprising a segment of at least 7 nucleotides that is exactly complementary to a subsequence from a reference sequence, except that the segment may or may not be exactly complementary at two interrogation positions. The nucleotides occupying the interrogation positions are selected by the following rules: (1) the first interrogation position is occupied by a different nucleotide in each of the four probes, (2) the second interrogation position is occupied by a different nucleotide in each of the four probes, (3) in first and second probes, the segment is exactly complementary to the subsequence, except at no more than one of the interrogation positions, (4) in third and fourth probes, the segment is exactly complementary to the subsequence, except at both of the interrogation positions.

In another aspect, the invention provides arrays of immobilized probes including helper mutations. Helper mutations are useful for, e.g., preventing self-annealing of probes having inverted repeats. In this strategy, the identity of a nucleotide in a target sequence is usually determined from a comparison of four probes. A first probe comprises a segment of at least 7 nucleotides exactly complementary to a subsequence of a reference sequence except at one or two positions, the segment including an interrogation position not at the one or two positions. The one or two positions are occupied by helper mutations.

Second, third and fourth mutant probes are each identical to a sequence comprising the wildtype probe or a subsequence thereof including the interrogation position and the one or two positions, except in the interrogation position, which is occupied by a different nucleotide in each of the four probes.

In another aspect, the invention provides arrays of probes comprising at least two probe sets, but lacking a probe set comprising probes that are perfectly matched to a reference sequence. Such arrays are usually employed in methods in which both reference and target sequence are hybridized to the array. The first probe set comprising a plurality of probes, each probe comprising a segment exactly complementary to a subsequence of at least 3 nucleotides of a reference sequence except at an interrogation position. The second probe set comprises a corresponding probe for each probe in the first probe set, the corresponding probe in the second probe set being identical to a sequence comprising the corresponding probe from the first probe set or a subsequence of at least three nucleotides thereof that includes the interrogation position, except that the interrogation position is occupied by a different nucleotide in each of the two corresponding probes and the complement to the reference sequence.

In another aspect, the invention provides methods of comparing a target sequence with a reference sequence comprising a predetermined sequence of nucleotides using any of the arrays described above. The methods comprise hybridizing the target nucleic acid to an array and determining which probes, relative to one another, in the array bind specifically to the target nucleic acid. The relative specific binding of the probes indicates whether the target sequence is the same or different from the reference sequence. In some such methods, the target sequence has a substituted nucleotide relative to the reference sequence in at least one undetermined position, and the relative specific binding of the probes indicates the location of the position and the nucleotide occupying the position in the target sequence. In some methods, a second target nucleic acid is also hybridized to the array. The relative specific binding of the probes then indicates both whether the target sequence is the same or different from the reference sequence, and whether the second target sequence is the same or different from the reference sequence. In some methods, when the array comprises two groups of probes tiled for first and second reference sequences, respectively, the relative specific binding of probes in the first group indicates whether the target sequence is the same or different from the first reference sequence. The relative specific binding of probes in the second group indicates whether the target sequence is the same or different from the second reference sequence.

Such methods are particularly useful for analyzing heterologous alleles of a gene. Some methods entail hybridizing both a reference sequence and a target sequence to any of the arrays of probes described above. Comparison of the relative specific binding of the probes to the reference and target sequences indicates whether the target sequence is the same or different from the reference sequence.

In another aspect, the invention provides arrays of immobilized probes in which the probes are designed to tile a reference sequence from a human immunodeficiency virus. Reference sequences from either the reverse transcriptase gene or protease gene of HIV are of particular interest. Some chips further comprise arrays of probes tiling a reference sequence from a 16S RNA or DNA encoding the 16S RNA from a pathogenic microorganism. The invention further provides methods of using such arrays in analyzing a HIV target sequence. The methods are particularly useful where the target sequence has a substituted nucleotide relative to the reference sequence in at least one position, the substitution conferring resistance to a drug use in treating a patient infected with a HIV virus. The methods reveal the existence of the substituted nucleotide. The methods are also particularly useful for analyzing a mixture of undetermined proportions of first and second target sequences from different HIV variants. The relative specific binding of probes indicates the proportions of the first and second target sequences.

In another aspect, the invention provides arrays of probes tiled based on reference sequence from a CFTR gene. An exemplary array comprises at least a group of probes comprising a wildtype probe, and five sets of three mutant probes. The wildtype probe is exactly complementary to a subsequence of a reference sequence from a cystic fibrosis gene, the segment having at least five interrogation positions corresponding to five contiguous nucleotides in the reference sequence. The probes in the first set of three mutant probes are each identical to the wildtype probe, except in a first of the five interrogation positions, which is occupied by a different nucleotide in each of the three mutant probes and the wildtype probe. The probes in the second set of three mutant probes are each identical to the wildtype probe, except in a second of the five interrogation positions, which is occupied by a different nucleotide in each of the three mutant probes and the wildtype probe. The probes in the third set of three mutant probes are each identical to the wildtype probe, except in a third of the five interrogation positions, which is occupied by a different nucleotide in each of the three mutant probes and the wildtype probe. The probes in the fourth set of three mutant probes are each identical to the wildtype probe, except in a fourth of the five interrogation positions, which is occupied by a different nucleotide in each of the three mutant probes and the wildtype probe. The probes in the fifth set of three mutant probes are each identical to the wildtype probe, except in a fifth of the five interrogation positions, which is occupied by a different nucleotide in each of the three mutant probes and the wildtype probe. A chip can comprise two such groups of probes. The first group comprises a wildtype probe exactly complementary to a first reference sequence, and the second group comprises a wildtype probe exactly complementary to a second reference sequence that is a mutated form of the first reference sequence.

The invention further provides methods of using the arrays of the invention for analyzing target sequences from a CFTR gene. The methods are capable of simultaneously analyzing first and second target sequences representing heterozygous alleles of a CFTR gene.

In another aspect, the invention provides arrays of probes tiling a reference sequence from a p53 gene, an hMLHl gene and/or an MSH2 gene. The invention further provides methods of using the arrays described above to analyze these genes. The method are useful, e.g., for diagnosing patients susceptible to developing cancer.

In another aspect, the invention provides arrays of probes tiling a reference sequence from a mitochondrial genome. The reference sequence may comprise part or all of the D-loop region, or all, or substantially all, of the mitochondrial genome. The invention further provides method of using the arrays described above to analyze target sequences from a mitochondrial genome. The methods are useful for identifying mutations associated with disease, and for forensic, epidemiological and evolutionary studies.

The invention provides a method for identifying proteins by differential labeling of peptides, the method comprising the following steps: (a) providing a sample comprising a polypeptide; (b) providing a plurality of labeling reagents which differ in molecular mass but do not differ in chromatographic retention properties and do not differ in ionization and detection properties in mass spectrographic analysis, wherein the differences in molecular mass are distinguishable by mass spectrographic analysis; (c) fragmenting the polypeptide into peptide fragments by enzymatic digestion or by non-enzymatic fragmentation; (d) contacting the labeling reagents of step (b) with the peptide fragments of step (c), thereby labeling the peptides with the differential labeling reagents; (e) separating the peptides by chromatography to generate an eluate; (f) feeding the eluate of step (e) into a mass spectrometer and quantifying the amount of each peptide and generating the sequence of each peptide by use of the mass spectrometer; (g) inputting the sequence to a computer program product which compares the inputted sequence to a database of polypeptide sequences to identify the polypeptide from which the sequenced peptide originated.

In one aspect, the sample of step (a) comprises a cell or a cell extract. The method can further comprise providing two or more samples comprising a polypeptide. One or more of the samples can be derived from a wild type cell and one sample can be derived from an abnormal or a modified cell. The abnormal cell can be a cancer cell. The modified cell can be a cell that is mutagenized &/or treated with a chemical, a physiological factor, or the presence of another organism (including, e.g. a eukaryotic organism, prokaryotic organism, virus, vector, prion, or part thereof), &/or exposed to an environmental factor or change or physical force (including, e.g., sound, light, heat, sonication, and radiation). The modification can be genetic change (including, for example, a change in DNA or RNA sequence or content) or otherwise.

In one aspect, the method further comprises purifying or fractionating the polypeptide before the fragmenting of step (c). The method can further comprise purifying or fractionating the polypeptide before the labeling of step (d). The method can further comprise purifying or fractionating the labeled peptide before the chromatography of step (e). In alternative aspects, the purifying or fractionating comprises a method selected from the group consisting of size exclusion chromatography, size exclusion chromatography, HPLC, reverse phase HPLC and affinity purification. In one aspect, the method further comprises contacting the polypeptide with a labeling reagent of step (b) before the fragmenting of step (c).

In one aspect, the labeling reagent of step (b) comprises the general formulae selected from the group consisting of: Z^{A}OH and Z^{B}OH, to esterify peptide C-terminals and/or Glu and Asp side chains; Z^{A}NH₂ and Z^{B}NH₂, to form amide bond with peptide C-terminals and/or Glu and Asp side chains; and Z^{A}CO₂H and Z^{B}CO₂H. to form amide bond with peptide N-terminals and/or Lys and Arg side chains; wherein Z^{A} and Z^{B} independently of one another comprise the general formula R-Z¹-A¹-Z²-A²-Z³-A³-Z⁴-A⁴-, Z¹, Z², Z³, and Z⁴ independently of one another, are selected from the group consisting of nothing, O, OC(O), OC(S), OC(O)O, OC(O)NR, OC(S)NR, OSiRR¹, S, SC(O), SC(S), SS, S(O), S(O₂), NR, NRR¹⁺, C(O), C(O)O, C(S), C(S)O, C(O)S, C(O)NR, C(S)NR, SiRR¹, (Si(RR¹)O)n, SNRR¹, Sn(RR¹)O, BR(OR¹), BRR¹, B(OR)(OR¹), OBR(OR¹), OBRR¹, and OB(OR)(OR¹), and R and R¹ is an alkyl group, A¹, A², A³, and A⁴ independently of one another, are selected from the group consisting of nothing or (CRR¹)n, wherein R, R¹, independently from other R and R¹ in Z¹ to Z⁴ and independently from other R and R¹ in A¹ to A⁴, are selected from the group consisting of a hydrogen atom, a halogen atom and an alkyl group; “n” in Z¹ to Z⁴, independent of n in A¹ to A⁴, is an integer having a value selected from the group consisting of 0 to about 51; 0 to about 41; 0 to about 31; 0 to about 21, 0 to about 11 and 0 to about 6.

In one aspect, the alkyl group (see definition below) is selected from the group consisting of an alkenyl, an alkynyl and an aryl group. One or more C—C bonds from (CRR¹)n can be replaced with a double or a triple bond; thus, in alternative aspects, an R or an R¹ group is deleted. The (CRR¹)n can be selected from the group consisting of an o-arylene, an m-arylene and a p-arylene, wherein each group has none or up to 6 substituents. The (CRR¹)n can be selected from the group consisting of a carbocyclic, a bicyclic and a tricyclic fragment, wherein the fragment has up to 8 atoms in the cycle with or without a heteroatom selected from the group consisting of an O atom, a N atom and an S atom.

In one aspect, two or more labeling reagents have the same structure but a different isotope composition. For example, in one aspect, Z^{A} has the same structure as Z^{B}, while Z^{A} has a different isotope composition than Z^{B}. In alternative aspects, the isotope is boron-10 and boron-11; carbon-12 and carbon-13; nitrogen-14 and nitrogen-15; and, sulfur-32 and sulfur-34. In one aspect, where the isotope with the lower mass is x and the isotope with the higher mass is y, and x and y are integers, x is greater than y.

In alternative aspects, x and y are between 1 and about 11, between 1 and about 21, between 1 and about 31, between 1 and about 41, or between 1 and about 51.

In one aspect, the labeling reagent of step (b) comprises the general formulae selected from the group consisting of: CD₃(CD₂)ₙOH/CH₃(CH₂)ₙOH, to esterify peptide C-terminals, where n=0, 1, 2 or y; CD₃(CD₂)ₙNH₂/CH₃(CH₂)ₙNH₂, to form amide bond with peptide C-terminals, where n=0, 1, 2 or y; and, D(CD₂)ₙCO₂H/H(CH₂)ₙCO₂H, to form amide bond with peptide N-terminals, where n=0, 1, 2 or y; wherein D is a deuteron atom, and y is an integer selected from the group consisting of about 51; about 41; about 31; about 21, about 11; about 6 and between about 5 and 51.

In one aspect, the labeling reagent of step (b) can comprise the general formulae selected from the group consisting of: Z^{A}OH and Z^{B}OH to esterify peptide C-terminals; Z^{A}NFH₂/Z^{B}NH₂ to form an amide bond with peptide C-terminals; and, Z^{A}CO₂H/Z^{B}CO₂H to form an amide bond with peptide N-terminals; wherein Z^{A} and Z^{B} have the general formula R-Z¹-A¹-Z²-A²-Z³-A³-Z⁴-A⁴-; Z¹, Z², Z³, and Z⁴, independently of one another, are selected from the group consisting of nothing, O, OC(O), OC(S), OC(O)O, OC(O)NR, OC(S)NR, OSiRR¹, S, SC(O), SC(S), SS, S(O), S(O₂), NR, NRR¹⁺, C(O), C(O)O, C(S), C(S)O, C(O)S, C(O)NR, C(S)NR, SiRR¹, (Si(RR¹)O)n, SnRR¹, Sn(RR¹)O, BR(OR¹), BRR¹, B(OR)(OR¹), OBR(OR¹), OBRR¹, and OB(OR)(OR¹); A¹, A², A³, and A⁴, independently of one another, are selected from the group consisting of nothing and the general formulae (CRR¹)n, and, R and R¹ is an alkyl group.

In one aspect, a single C—C bond in a (CRR¹)n group is replaced with a double or a triple bond; thus, the R and R¹ can be absent. The (CRR¹)n can comprise a moiety selected from the group consisting of an o-arylene, an m-arylene and a p-arylene, wherein the group has none or up to 6 substituents. The group can comprise a carbocyclic, a bicyclic, or a tricyclic fragments with up to 8 atoms in the cycle, with or without a heteroatom selected from the group consisting of an O atom, an N atom and an S atom. In one aspect, R, R¹, independently from other R and R¹ in Z¹-Z⁴ and independently from other R and R¹ in A¹-A⁴, are selected from the group consisting of a hydrogen atom, a halogen and an alkyl group. The alkyl group (see definition below) can be an alkenyl, an alkynyl or an aryl group.

In one aspect, the “n” in Z¹-Z⁴ is independent of n in A¹-A⁴ and is an integer selected from the group consisting of about 51; about 41; about 31; about 21, about 11 and about 6. In one aspect, Z^{A} has the same structure a Z^{B} but Z^{A} further comprises x number of —CH₂— fragment(s) in one or more A¹-A⁴ fragments, wherein x is an integer. In one aspect, Z^{A} has the same structure a Z^{B} but Z^{A} further comprises x number of —CF₂— fragment(s) in one or more A¹-A⁴ fragments, wherein x is an integer. In one aspect, Z^{A} comprises x number of protons and Z^{B} comprises y number of halogens in the place of protons, wherein x and y are integers. In one aspect, Z^{A} contains x number of protons and Z^{B} contains y number of halogens, and there are x-y number of protons remaining in one or more A¹-A⁴ fragments, wherein x and y are integers. In one aspect, Z^{A} further comprises x number of —O— fragment(s) in one or more A¹-A⁴ fragments, wherein x is an integer. In one aspect, Z^{A} further comprises x number of —S— fragment(s) in one or more A¹-A⁴ fragments, wherein x is an integer. In one aspect, Z^{A} further comprises x number of —O— fragment(s) and Z^{B} further comprises y number of —S— fragment(s) in the place of —O— fragment(s), wherein x and y are integers. In one aspect, Z^{A} further comprises x-y number of —O— fragment(s) in one or more A¹-A⁴ fragments, wherein x and y are integers.

In alternative aspects, x and y are integers selected from the group consisting of between 1 about 51; between 1 about 41; between 1 about 31; between 1 about 21, between I about 11 and between 1 about 6, wherein x is greater than y.

In one aspect, the labeling reagent of step (b) comprises the general formulae selected from the group consisting of: CH₃(CH₂)ₙOH/CH₃(CH₂)ₙ₊ₘOH, to esterify peptide C-terminals, where n=0, 1, 2, . . . , y; m=1, 2, . . . , y; CH₃(CH₂)ₙ NH₂/CH₃(CH₂)ₙ₊ₘNH₂, to form amide bond with peptide C-terminals, where n=0, 1, 2, . . . , y; m=1, 2, . . . , y; and, H(CH₂)ₙCO₂H/H(CH₂)ₙ₊ₘCO₂H, to form amide bond with peptide N-terminals, where n=0, 1, 2, . . . , y; m=1, 2, . . . , y; wherein n, m and y are integers. In one aspect, n, m and y are integers selected from the group consisting of about 51; about 41; about 31; about 21, about 11; about 6 and between about 5 and 51.

In one aspect, the separating of step (e) comprises a liquid chromatography system, such as a multidimensional liquid chromatography or a capillary chromatography system. In one aspect, the mass spectrometer comprises a tandem mass spectrometry device. In one aspect, the method further comprises quantifying the amount of each polypeptide or each peptide.

The invention provides a method for defining the expressed proteins associated with a given cellular state, the method comprising the following steps: (a) providing a sample comprising a cell in the desired cellular state; (b) providing a plurality of labeling reagents which differ in molecular mass but do not differ in chromatographic retention properties and do not differ in ionization and detection properties in mass spectrographic analysis, wherein the differences in molecular mass are distinguishable by mass spectrographic analysis; (c) fragmenting polypeptides derived from the cell into peptide fragments by enzymatic digestion or by non-enzymatic fragmentation; (d) contacting the labeling reagents of step (b) with the peptide fragments of step (c), thereby labeling the peptides with the differential labeling reagents; (e) separating the peptides by chromatography to generate an eluate; (f) feeding the eluate of step (e) into a mass spectrometer and quantifying the amount of each peptide and generating the sequence of each peptide by use of the mass spectrometer; (g) inputting the sequence to a computer program product which compares the inputted sequence to a database of polypeptide sequences to identify the polypeptide from which the sequenced peptide originated, thereby defining the expressed proteins associated with the cellular state.

The invention provides a method for quantifying changes in protein expression between at least two cellular states, the method comprising the following steps: (a) providing at least two samples comprising cells in a desired cellular state; (b) providing a plurality of labeling reagents which differ in molecular mass but do not differ in chromatographic retention properties and do not differ in ionization and detection properties in mass spectrographic analysis, wherein the differences in molecular mass are distinguishable by mass spectrographic analysis; (c) fragmenting polypeptides derived from the cells into peptide fragments by enzymatic digestion or by non-enzymatic fragmentation; (d) contacting the labeling reagents of step (b) with the peptide fragments of step (c), thereby labeling the peptides with the differential labeling reagents, wherein the labels used in one same are different from the labels used in other samples; (e) separating the peptides by chromatography to generate an eluate; (f) feeding the eluate of step (e) into a mass spectrometer and quantifying the amount of each peptide and generating the sequence of each peptide by use of the mass spectrometer; (g) inputting the sequence to a computer program product which identifies from which sample each peptide was derived, compares the inputted sequence to a database of polypeptide sequences to identify the polypeptide from which the sequenced peptide originated, and compares the amount of each polypeptide in each sample, thereby quantifying changes in protein expression between at least two cellular states.

The invention provides a method for identifying proteins by differential labeling of peptides, the method comprising the following steps: (a) providing a sample comprising a polypeptide; (b) providing a plurality of labeling reagents which differ in molecular mass but do not differ in chromatographic retention properties and do not differ in ionization and detection properties in mass spectrographic analysis, wherein the differences in molecular mass are distinguishable by mass spectrographic analysis; (c) fragmenting the polypeptide into peptide fragments by enzymatic digestion or by non-enzymatic fragmentation; (d) contacting the labeling reagents of step (b) with the peptide fragments of step (c), thereby labeling the peptides with the differential labeling reagents; (e) separating the peptides by multidimensional liquid chromatography to generate an eluate; (f) feeding the eluate of step (e) into a tandem mass spectrometer and quantifying the amount of each peptide and generating the sequence of each peptide by use of the mass spectrometer; (g) inputting the sequence to a computer program product which compares the inputted sequence to a database of polypeptide sequences to identify the polypeptide from which the sequenced peptide originated.

The invention provides a chimeric labeling reagent comprising (a) a first domain comprising a biotin; and (b) a second domain comprising a reactive group capable of covalently binding to an amino acid, wherein the chimeric labeling reagent comprises at least one isotope. The isotope(s) can be in the first domain or the second domain. For example, the isotope(s) can be in the biotin.

In alternative aspects, the isotope can be a deuterium isotope, a boron-10 or boron-11 isotope, a carbon-12 or a carbon-13 isotope, a nitrogen-14 or a nitrogen-15 isotope, or, a sulfur-32 or a sulfur-34 isotope. The chimeric labeling reagent can comprise two or more isotopes. The chimeric labeling reagent reactive group capable of covalently binding to an amino acid can be a succimide group, an isothiocyanate group or an isocyanate group. The reactive group can be capable of covalently binding to an amino acid binds to a lysine or a cysteine.

The chimeric labeling reagent can further comprising a linker moiety linking the biotin group and the reactive group. The linker moiety can comprise at least one isotope. In one aspect, the linker is a cleavable moiety that can be cleaved by, e.g., enzymatic digest or by reduction.

The invention provides a method of comparing relative protein concentrations in a sample comprising (a) providing a plurality of differential small molecule tags, wherein the small molecule tags are structurally identical but differ in their isotope composition, and the small molecules comprise reactive groups that covalently bind to cysteine or lysine residues or both; (b) providing at least two samples comprising polypeptides; (c) attaching covalently the differential small molecule tags to amino acids of the polypeptides; (d) determining the protein concentrations of each sample in a tandem mass spectrometer; and, (d) comparing relative protein concentrations of each sample. In one aspect, the sample comprises a complete or a fractionated cellular sample.

In one aspect of the method, the differential small molecule tags comprise a chimeric labeling reagent comprising (a) a first domain comprising a biotin; and, (b) a second domain comprising a reactive group capable of covalently binding to an amino acid, wherein the chimeric labeling reagent comprises at least one isotope. The isotope can be a deuterium isotope, a boron-10 or boron-11 isotope, a carbon-12 or a carbon-13 isotope, a nitrogen-14 or a nitrogen-15 isotope, or, a sulfur-32 or a sulfur-34 isotope. The chimeric labeling reagent can comprise two or more isotopes. The reactive group can be capable of covalently binding to an amino acid is selected from the group consisting of a succimide group, an isothiocyanate group and an isocyanate group.

The invention provides a method of comparing relative protein concentrations in a sample comprising (a) providing a plurality of differential small molecule tags, wherein the differential small molecule tags comprise a chimeric labeling reagent comprising (i) a first domain comprising a biotin; and, (ii) a second domain comprising a reactive group capable of covalently binding to an amino acid, wherein the chimeric labeling reagent comprises at least one isotope; (b) providing at least two samples comprising polypeptides; (c) attaching covalently the differential small molecule tags to amino acids of the polypeptides; (d) isolating the tagged polypeptides on a biotin-binding column by binding tagged polypeptides to the column, washing non-bound materials off the column, and eluting tagged polypeptides off the column; (e) determining the protein concentrations of each sample in a tandem mass spectrometer; and, (f) comparing relative protein concentrations of each sample.

The details of one or more embodiments of the invention are set forth in the accompanying drawings and the description below. Other features, objects, and advantages of the invention will be apparent from the description and drawings, and from the claims.

All publications, patents and patent applications cited herein are hereby expressly incorporated by reference for all purposes.

### DESCRIPTION OF DRAWINGS

The following drawings are illustrative of aspects of the invention and are not meant to limit the scope of the invention as encompassed by the claims.

FIG. 1 illustrates an exemplary process of the invention wherein samples are combined, separated by multidimensional chromatography, and analyzed by mass spectrometry methods, as described in detail, below.

FIG. 2 is an illustration of a MALDI MS spectrum of a peptide pairs, as described in detail, below.

FIG. 3 illustrates an exemplary 3D LC set-up and process, as described in detail, below.

Like reference symbols in the various drawings indicate like elements.

### DETAILED DESCRIPTION

Specific Strategies for Utilizing Nucleic Acid Arrays

The invention provides a number of strategies for comparing a polynucleotide of known sequence (a reference sequence) with variants of that sequence (target sequences).

The comparison can be performed at the level of entire genomes, chromosomes, genes, exons or introns, or can focus on individual mutant sites and immediately adjacent bases. The strategies allow detection of variations, such as mutations or polymorphisms, in the target sequence irrespective whether a particular variant has previously been characterized. The strategies both define the nature of a variant and identify its location in a target sequence.

The strategies employ arrays of oligonucleotide probes immobilized to a solid support. Target sequences are analyzed by determining the extent of hybridization at particular probes in the array. The strategy in selection of probes facilitates distinction between perfectly matched probes and probes showing single-base or other degrees of mismatches.

The strategy usually entails sampling each nucleotide of interest in a target sequence several times, thereby achieving a high degree of confidence in its identity. This level of confidence is further increased by sampling of adjacent nucleotides in the target sequence to nucleotides of interest.

The number of probes on the chip can be quite large (e.g., 10⁵-10⁶). However, usually only a small proportion of the total number of probes of a given length are represented. Some advantage of the use of only a small proportion of all possible probes of a given length include: (i) each position in the array is highly informative, whether or not hybridization occurs; (ii) nonspecific hybridization is minimized; (iii) it is straightforward to correlate hybridization differences with sequence differences, particularly with reference to the hybridization pattern of a known standard; and (iv) the ability to address each probe independently during synthesis, using high resolution photolithography, allows the array to be designed and optimized for any sequence. For example the length of any probe can be varied independently of the others.

The present tiling strategies result in sequencing and comparison methods suitable for routine large-scale practice with a high degree of confidence in the sequence output.

General Tiling Strategies

Selection of Reference Sequence

The chips can be designed to contain probes exhibiting complementarity to one or more selected reference sequence whose sequence is known. The chips are used to read a target sequence comprising either the reference sequence itself or variants of that sequence. Target sequences may differ from the reference sequence at one or more positions but show a high overall degree of sequence identity with the reference sequence (e.g., at least 75, 90, 95, 99, 99.9 or 99-99%). Any polynucleotide of known sequence can be selected as a reference sequence. Reference sequences of interest include sequences known to include mutations or polymorphisms associated with phenotypic changes having clinical significance in human patients. For example, the CFTR gene and P53 gene in humans have been identified as the location of several mutations resulting in cystic fibrosis or cancer respectively. Other reference sequences of interest include those that serve to identify pathogenic microorganisms and/or are the site of mutations by which such microorganisms acquire drug resistance (e.g., the HIV reverse transcriptase gene). Other reference sequences of interest include regions where polymorphic variations are known to occur (e.g., the D-loop region of mitochondrial DNA). These reference sequences have utility for, e.g., forensic or epidemiological studies. Other reference sequences of interest include p34 (related to p53), p65 (implicated in breast, prostate and liver cancer), and DNA segments encoding cytochromes P450 (see Meyer et al., Pharmac. Ther. 46, 349-355 (1990)). Other reference sequences of interest include those from the genome of pathogenic viruses (e.g., hepatitis J, B, or Q, herpes virus (e.g., VZV, HSV-1, HAV-6, HSV-II, and CMV, Epstein Barr virus), adenovirus, influenza virus, flaviviruses, echovirus, rhinovirus, coxsackie virus, cornovirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus. Other reference sequences of interest are from genomes or episomes of pathogenic bacteria, particularly regions that confer drug resistance or allow phylogenic characterization of the host (e.g., 16S rRNA or corresponding DNA). For example, such bacteria include Chlamydia, rickettsial bacteria, mycobacteria, staphylococci, streptococci, pneumonococci, meningococci and conococci, klebsiella, proteus, serratia, pseudomonas, legionella, diphtheria, salmonella, bacilli, cholera, tetanus, botulism, anthrax, plague, leptospirosis, and Lymes disease bacteria. Other reference sequences of interest include those in which mutations result in the following autosomal recessive disorders: sickle cell anemia, beta-thalassemia, phenylketonuria, galactosemia, Wilson's disease, hemochromatosis, severe combined immunodeficiency, alpha-1-antitrypsin deficiency, albinism, alkaptonuria, lysosomal storage diseases and Ehlers-Danlos syndrome. Other reference sequences of interest include those in which mutations result in X-linked recessive disorders: hemophilia, glucose-6-phosphate dehydrogenase, agammaglobulemia, diabetes insipidus, Lesch-Nyhan syndrome, muscular dystrophy, Wiskott-Aldrich syndrome, Fabry's disease and fragile X-syndrome. Other reference sequences of interest includes those in which mutations result in the following autosomal dominant disorders: familial hypercholesterolemia, polycystic kidney disease, Huntingdon's disease, hereditary spherocytosis, Marfan's syndrome, von Willebrand's disease, neurofibromatosis, tuberous sclerosis, hereditary hemorrhagic telangiectasia, familial colonic polyposis, Ehlers-Danlos syndrome, myotonic dystrophy, muscular dystrophy, osteogenesis imperfecta, acute intermittent porphyria, and von Hippel-Lindau disease.

The length of a reference sequence can vary widely from a full-length genome, to an individual chromosome, episome, gene, component of a gene, such as an exon, intron or regulatory sequences, to a few nucleotides. A reference sequence of between about 2, 5, 10, 20, 50, 100, 5000, 1000, 5,000 or 10,000, 20,000 or 100,000 nucleotides is common.

Sometimes only particular regions of a sequence (e.g., exons of a gene) are of interest. In such situations, the particular regions can be considered as separate reference sequences or can be considered as components of a single reference sequence, as matter of arbitrary choice.

A reference sequence can be any naturally occurring, mutant, consensus or purely hypothetical sequence of nucleotides, RNA or DNA. For example, sequences can be obtained from computer data bases, publications or can be determined or conceived de novo. Usually, a reference sequence is selected to show a high degree of sequence identity to envisaged target sequences. Often, particularly, where a significant degree of divergence is anticipated between target sequences, more than one reference sequence is selected. Combinations of wildtype and mutant reference sequences are employed in several applications of the tiling strategy.

Chip Design

Basic Tiling Strategy

The basic tiling strategy provides an array of immobilized probes for analysis of target sequences showing a high degree of sequence identity to one or more selected reference sequences. The strategy is first illustrated for an exemplary array that is subdivided into four probe sets, although it will be apparent that in some situations, satisfactory results are obtained from only two probe sets. A first probe set comprises a plurality of probes exhibiting perfect complementarity with a selected reference sequence. The perfect complementarity usually exists throughout the length of the probe. However, probes having a segment or segments of perfect complementarity that is/are flanked by leading or trailing sequences lacking complementarity to the reference sequence can also be used. Within a segment of complementarity, each probe in the first probe set has at least one interrogation position that corresponds to a nucleotide in the reference sequence. That is, the interrogation position is aligned with the corresponding nucleotide in the reference sequence, when the probe and reference sequence are aligned to maximize complementarity between the two. If a probe has more than one interrogation position, each corresponds with a respective nucleotide in the reference sequence. The identity of an interrogation position and corresponding nucleotide in a particular probe in the first probe set cannot be determined simply by inspection of the probe in the first set. As will become apparent, an interrogation position and corresponding nucleotide is defined by the comparative structures of probes in the first probe set and corresponding probes from additional probe sets.

A probe can have an interrogation position at each position in the segment complementary to the reference sequence. An interrogation position can be located away from the ends of a segment of complementarity. Interrogation positions may provide more accurate data when located away from the ends of a segment of complementarity. A probe can have a segment of complementarity of length x does not contain more than x-2 interrogation positions. Since probes are typically 9-21 nucleotides, and usually all of a probe is complementary, a probe typically has 1-19 interrogation positions. The probes can contain a single interrogation position, at or near the center of probe.

For each probe in the first set, there can be three corresponding probes from three additional probe sets. Thus, there can be four probes corresponding to each nucleotide of interest in the reference sequence. Each of the four corresponding probes has an interrogation position aligned with that nucleotide of interest. The probes from the three additional probe sets can be identical to the corresponding probe from the first probe set with one exception. The exception is that at least one (and often only one) interrogation position, which occurs in the same position in each of the four corresponding probes from the four probe sets, is occupied by a different nucleotide in the four probe sets. For example, for an A nucleotide in the reference sequence, the corresponding probe from the first probe set has its interrogation position occupied by a T, and the corresponding probes from the additional three probe sets have their respective interrogation positions occupied by A, C, or G, a different nucleotide in each probe. Of course, if a probe from the first probe set comprises trailing or flanking sequences lacking complementarity to the reference sequences, these sequences need not be present in corresponding probes from the three additional sets. Likewise corresponding probes from the three additional sets can contain leading or trailing sequences outside the segment of complementarity that are not present in the corresponding probe from the first probe set. Occasionally, the probes from the additional three probe set are identical (with the exception of interrogation position(s)) to a contiguous subsequence of the full complementary segment of the corresponding probe from the first probe set. In this case, the subsequence includes the interrogation position and usually differs from the full-length probe only in the omission of one or both terminal nucleotides from the termini of a segment of complementarity.

That is, if a probe from the first probe set has a segment of complementarity of length n, corresponding probes from the other sets will usually include a subsequence of the segment of at least length n-2. Thus, the subsequence is usually at least 3, 4, 7, 9, 15, 21, or 25 nucleotides long, most typically, in the range of 9-21 nucleotides. The subsequence should be sufficiently long to allow a probe to hybridize detectably more strongly to a variant of the reference sequence mutated at the interrogation position than to the reference sequence.

The probes can be oligodeoxyribonucleotides or oligoribonucleotides, or any modified forms of these polymers that are capable of hybridizing with a target nucleic sequence by complementary base-pairing. Complementary base pairing means sequence-specific base pairing which includes e.g., Watson-Crick base pairing as well as other forms of base pairing such as Hoogsteen base pairing. Modified forms include 2′-0-methyl oligoribonucleotides and so-called PNAs, in which oligodeoxyribonucleotides are linked via peptide bonds rather than phophodiester bonds. The probes can be attached by any linkage to a support (e.g., 3′, 5′ or via the base). 3′ attachment is more usual as this orientation is compatible with a chemistry for solid phase synthesis of oligonucleotides.

The number of probes in the first probe set (and as a consequence the number of probes in additional probe sets) depends on the length of the reference sequence, the number of nucleotides of interest in the reference sequence and the number of interrogation positions per probe. In general, each nucleotide of interest in the reference sequence requires the same interrogation position in the four sets of probes. A reference sequence can have 100 nucleotides, 50 of which are of interest, and probes each having a single interrogation position. In this situation, the first probe set requires fifty probes, each having one interrogation position corresponding to a nucleotide of interest in the reference sequence. The second, third and fourth probe sets each have a corresponding probe for each probe in the first probe set, and so each also contains a total of fifty probes. The identity of each nucleotide of interest in the reference sequence is determined by comparing the relative hybridization signals at four probes having interrogation positions corresponding to that nucleotide from the four probe sets.

In some reference sequences, every nucleotide is of interest. In other reference sequences, only certain portions in which variants (e.g., mutations or polymorphisms) are concentrated are of interest. In other reference sequences, only particular mutations or polymorphisms and immediately adjacent nucleotides are of interest. Usually, the first probe set has interrogation positions selected to correspond to at least a nucleotide (e.g., representing a point mutation) and one immediately adjacent nucleotide. Usually, the probes in the first set have interrogation positions corresponding to at least 3, 10, 50, 100, 1000, or 20,000 contiguous nucleotides. The probes usually have interrogation positions corresponding to at least 5, 10, 30, 50, 75, 90, 99 or sometimes 100% of the nucleotides in a reference sequence.

The probes in the first probe set can completely span the reference sequence and overlap with one another relative to the reference sequence. For example, in one common arrangement each probe in the first probe set differs from another probe in that set by the omission of a 3′ base complementary to the reference sequence and the acquisition of a 5′ base complementary to the reference sequence.

The probes in a set can be arranged in order of the sequence in a lane across the chip. A lane contains a series of overlapping probes, which represent or tile across, the selected reference sequence. The components of the four sets of probes are usually laid down in four parallel lanes, collectively constituting a row in the horizontal direction and a series of 4-member columns in the vertical direction. Corresponding probes from the four probe sets (i.e., complementary to the same subsequence of the reference sequence) occupy a column.

Each probe in a lane usually differs from its predecessor in the lane by the omission of a base at one end and the inclusion of additional base at the other end. However, this orderly progression of probes can be interrupted by the inclusion of control probes or omission of probes in certain columns of the array. Such columns serve as controls to orient the chip, or gauge the background, which can include target sequence nonspecifically bound to the chip.

The probes sets can be laid down in lanes such that all probes having an interrogation position occupied by an A form an-A-lane, all probes having an interrogation position occupied by a C form a C-lane, all probes having an interrogation position occupied by a G form a G-lane, and all probes having an interrogation position occupied by a T (or U) form a T lane (or a U lane). Note that in this arrangement there is not a unique correspondence between probe sets and lanes. Thus, the probe from the first probe set is laid down in the A-lane, C-lane, A-lane, A-lane and T-lane for the five columns. The interrogation position on a column of probes corresponds to the position in the target sequence whose identity is determined from analysis of hybridization to the probes in that column. The interrogation position can be anywhere in a probe but is usually at or near the central position of the probe to maximize differential hybridization signals between a perfect match and a single-base mismatch. For example, for an 11 mer probe, the central position is the sixth nucleotide.

Although the array of probes is usually laid down in rows and columns as described above, such a physical arrangement of probes on the chip is not essential. Provided that the spatial location of each probe in an array is known, the data from the probes can be collected and processed to yield the sequence of a target irrespective of the physical arrangement of the probes on a chip. In processing the data, the hybridization signals from the respective probes can be reasserted into any conceptual array desired for subsequent data reduction whatever the physical arrangement of probes on the chip.

A range of lengths of probes can be employed in the chips. As noted above, a probe may consist exclusively of a complementary segments, or may have one or more complementary segments juxtaposed by flanking, trailing and/or intervening segments. In the latter situation, the total length of complementary segment(s) is more important than the length of the probe. In functional terms, the complementarity segment(s) of the first probe sets should be sufficiently long to allow the probe to hybridize detectably more strongly to a reference sequence compared with a variant of the reference including a single base mutation at the nucleotide corresponding to the interrogation position of the probe.

Similarly, the complementarity segment(s) in corresponding probes from additional probe sets can be sufficiently long to allow a probe to hybridize detectably more strongly to a variant of the reference sequence having a single nucleotide substitution at the interrogation position relative to the reference sequence. A probe can have a single complementary segment having a length of at least 3 nucleotides, and more usually at least 5, 6, 7, 8, 9, 10, 11, 12, 13, 14, 15, 16, 17, 18, 19, 20, 21, 22, 23, 24, 25 or bases exhibiting perfect complementarity (other than possibly at the interrogation position(s) depending on the probe set) to the reference sequence. In bridging strategies, where more than one segment of complementarity is present, each segment provides at least three complementary nucleotides to the reference sequence and the combined segments provide at least two segments of three or a total of six complementary nucleotides. As in the other strategies, the combined length of complementary segments is typically from 6-30 nucleotides, or, from about 9-21 nucleotides. The two segments are often approximately the same length. Often, the probes (or segment of complementarity within probes) have an odd number of bases, so that an interrogation position can occur in the exact center of the probe.

In some chips, all probes are the same length. Other chips employ different groups of probe sets, in which case the probes are of the same size within a group, but differ between different groups. For example, some chips have one group comprising four sets of probes as described above in which all the probes are 11 mers, together with a second group comprising four sets of probes in which all of the probes are 13 mers. Of course, additional groups of probes can be added.

Thus, some chips contain, e.g., four groups of probes having sizes of 11 mers, 13 mers, 15 mers and 17 mers. Other chips have different size probes within the same group of four probe sets. In these chips, the probes in the first set can vary in length independently of each other. Probes in the other sets are usually the same length as the probe occupying the same column from the first set. However, occasionally different lengths of probes can be included at the same column position in the four lanes. The different length probes are included to equalize hybridization signals from probes irrespective of whether A-T or C-G bonds are formed at the interrogation position.

The length of probe can be important in distinguishing between a perfectly matched probe and probes showing a single-base mismatch with the target sequence. The discrimination is usually greater for short probes. Shorter probes are usually also less susceptible to formation of secondary structures. However, the absolute amount of target sequence bound, and hence the signal, is greater for larger probes. The probe length representing the optimum compromise between these competing considerations may vary depending on inter alia the GC content of a particular region of the target DNA sequence, secondary structure, synthesis efficiency and cross-hybridization. In some regions of the target, depending on hybridization conditions, short probes (e.g., 11 mers) may provide information that is inaccessible from longer probes (e.g., 19 mers) and vice versa. Maximum sequence information can be read by including several groups of different sized probes on the chip as noted above. However, for many regions of the target sequence, such a strategy provides redundant information in that the same sequence is read multiple times from the different groups of probes. Equivalent information can be obtained from a single group of different sized probes in which the sizes are selected to maximize readable sequence at particular regions of the target sequence. The strategy of customizing probe length within a single group of probe sets minimizes the total number of probes required to read a particular target sequence. This leaves ample capacity for the chip to include probes to other reference sequences.

The invention provides an optimization block which allows systematic variation of probe length and interrogation position to optimize the selection of probes for analyzing a particular nucleotide in a reference sequence. The block comprises alternating columns of probes complementary to the wildtype target and probes complementary to a specific mutation. The interrogation position is varied between columns and probe length is varied down a column.

Hybridization of the chip to the reference sequence or the mutant form of the reference sequence identifies the probe length and interrogation position providing the greatest differential hybridization signal.

The probes are designed to be complementary to either strand of the reference sequence (e.g., coding or non-coding). some chips contain separate groups of probes, one complementary to the coding strand, the other complementary to the noncoding strand. Independent analysis of coding and noncoding strands provides largely redundant information. However, the regions of ambiguity in reading the coding strand are not always the same as those in reading the noncoding strand. Thus, combination of the information from coding and noncoding strands increases the overall accuracy of sequencing.

Some chips contain additional probes or groups of probes designed to be complementary to a second reference sequence. The second reference sequence can often be a subsequence of the first reference sequence bearing one or more commonly occurring mutations or interstrain variations. The second group of probes is designed by the same principles as described above except that the probes exhibit complementarity to the second reference sequence. The inclusion of a second group is particular useful for analyzing short subsequences of the primary reference sequence in which multiple mutations are expected to occur within a short distance commensurate with the length of the probes (i.e., two or more mutations within 9 to 21 bases). Of course, the same principle can be extended to provide chips containing groups of probes for any number of reference sequences. Alternatively, the chips may contain additional probe(s) that do not form part of a tiled array as noted above, but rather serves as probe(s) for a conventional reverse dot blot. For example, the presence of mutation can be detected from binding of a target sequence to a single oligomeric probe harboring the mutation. An additional probe containing the equivalent region of the wildtype sequence can be included as a control.

The chips can be read by comparing the intensities of labeled target bound to the probes in an array. In one aspect, a comparison is performed between each lane of probes (e.g., A, C, G and T lanes) at each columnar position (physical or conceptual). For a particular columnar position, the lane showing the greatest hybridization signal is called as the nucleotide present at the position in the target sequence corresponding to the interrogation position in the probes. The corresponding position in the target sequence is that aligned with the interrogation position in corresponding probes when the probes and target are aligned to maximize complementarity. Of the four probes in a column, only one can exhibit a perfect match to the target sequence whereas the others usually exhibit at least a one base pair mismatch. The probe exhibiting a perfect match usually produces a substantially greater hybridization signal than the other three probes in the column and is thereby easily identified. However, in some regions of the target sequence, the distinction between a perfect match and a one-base mismatch is less clear. Thus, a call ratio is established to define the ratio of signal from the best hybridizing probes to the second best hybridizing probe that must be exceeded for a particular target position to be read from the probes. A high call ratio ensures that few if any errors are made in calling target nucleotides, but can result in some nucleotides being scored as ambiguous, which could in fact be accurately read.

A lower call ratio can result in fewer ambiguous calls, but can result in more erroneous calls. It has been found that at a call ratio of 1.2 virtually all calls are accurate. However, a small but significant number of bases (e.g., up to about %) may have to be scored as ambiguous.

Although small regions of the target sequence can sometimes be ambiguous, these regions usually occur at the same or similar segments in different target sequences. Thus, for pre-characterized mutations, it is known in advance whether that mutation is likely to occur within a region of unambiguously determinable sequence.

An array of probes is most useful for analyzing the reference sequence from which the probes were designed and variants of that sequence exhibiting substantial sequence similarity with the reference sequence (e.g., several single-base mutants spaced over the reference sequence). When an array is used to analyze the exact reference sequence from which it was designed, one probe exhibits a perfect match to the reference sequence, and the other three probes in the same column exhibits single-base mismatches. Thus, discrimination between hybridization signals is usually high and accurate sequence is obtained. High accuracy is also obtained when an array is used for analyzing a target sequence comprising a variant of the reference sequence that has a single mutation relative to the reference sequence, or several widely spaced mutations relative to the reference sequence. At different mutant loci, one probe exhibits a perfect match to the target, and the other three probes occupying the same column exhibit single-base mismatches, the difference (with respect to analysis of the reference sequence) being the lane in which the perfect match occurs.

For target sequences showing a high degree of divergence from the reference strain or incorporating several closely spaced mutations from the reference strain, a single group of probes (i.e., designed with respect to a single reference sequence) will not always provide accurate sequence for the highly variant region of this sequence. At some particular columnar positions, it may be that no single probe exhibits perfect complementarity to the target and that any comparison must be based on different degrees of mismatch between the four probes. Such a comparison does not always allow the target nucleotide corresponding to that columnar position to be called. Deletions in target sequences can be detected by loss of signal from probes having interrogation positions encompassed by the deletion. However, signal may also be lost from probes having interrogation positions closely proximal to the deletion resulting in some regions of the target sequence that cannot be read. Target sequence bearing insertions will also exhibit short regions including and proximal to the insertion that usually cannot be read.

The presence of short regions of difficult-to-read target because of closely spaced mutations, insertions or deletion, does not prevent determination of the remaining sequence of the target as different regions of a target sequence are determined independently. Moreover, such ambiguities as might result from analysis of diverse variants with a single group of probes can be avoided by including multiple groups of probe sets on a chip. For example, one group of probes can be designed based on a full-length reference sequence, and the other groups on subsequences of the reference sequence incorporating frequently occurring mutations or strain variations.

In one aspect, the sequencing strategy of the invention has the capacity to simultaneously detect and quantify proportions of multiple target sequences. Such capacity is valuable, e.g., for diagnosis of patients who are heterozygous with respect to a gene or who are infected with a virus, such as HIV, which is usually present in several polymorphic forms. Such capacity is also useful in analyzing targets from biopsies of tumor cells and surrounding tissues. The presence of multiple target sequences is detected from the relative signals of the four probes at the array columns corresponding to the target nucleotides at which diversity occurs. The relative signals at the four probes for the mixture under test are compared with the corresponding signals from a homogeneous reference sequence. An increase in a signal from a probe that is mismatched with respect to the reference sequence, and a corresponding decrease in the signal from the probe which is matched with the reference sequence signal the presence of a mutant strain in the mixture. The extent in shift in hybridization signals of the probes is related to the proportion of a target sequence in the mixture. Shifts in relative hybridization signals can be quantitatively related to proportions of reference and mutant sequence by prior calibration of the chip with seeded mixtures of the mutant and reference sequences. By this means, a chip can be used to detect variant or mutant strains constituting as little as 1, 5, 20, or 25% of a mixture of stains.

Similar principles allow the simultaneous analysis of multiple target sequences even when none is identical to the reference sequence. For example, with a mixture of two target sequences bearing first and second mutations, there would be a variation in the hybridization patterns of probes having interrogation positions corresponding to the first and second mutations relative to the hybridization pattern with the reference sequence. At each position, one of the probes having a mismatched interrogation position relative to the reference sequence would show an increase in hybridization signal, and the probe having a matched interrogation position relative to the reference sequence would show a decrease in hybridization signal. Analysis of the hybridization pattern of the mixture of mutant target sequences, in some aspect, in comparison with the hybridization pattern of the reference sequence, indicates the presence of two mutant target sequences, the position and nature of the mutation in each strain, and the relative proportions of each strain.

In a variation of the above method, the different components in a mixture of target sequences are differentially labeled before being applied to the array. For example, a variety of fluorescent labels emitting at different wavelength are available. The use of differential labels allows independent analysis of different targets bound simultaneously to the array. For example, the methods permit comparison of target sequences obtained from a patient at different stages of a disease.

Omission of Probes

The general strategy of the aspects of the invention outlined above employs four probes to read each nucleotide of interest in a target sequence. One probe (from the first probe set) shows a perfect match to the reference sequence and the other three probes (from the second, third and fourth probe sets) exhibit a mismatch with the reference sequence and a perfect match with a target sequence bearing a mutation at the nucleotide of interest.

The provision of three probes from the second, third and fourth probe sets allows detection of each of the three possible nucleotide substitutions of any nucleotide of interest. However, in some reference sequences or regions of reference sequences, it is known in advance that only certain mutations are likely to occur. Thus, for example, at one site it might be known that an A nucleotide in the reference sequence may exist as a T mutant in some target sequences but is unlikely to exist as a C or G mutant. Accordingly, for analysis of this region of the reference sequence, one might include only the first and second probe sets, the first probe set exhibiting perfect complementarity to the reference sequence, and the second probe set having an interrogation position occupied by an invariant A residue (for detecting the T mutant). In other situations, one might include the first, second and third probes sets (but not the fourth) for detection of a wildtype nucleotide in the reference sequence and two mutant variants thereof in target sequences. In some chips, probes that would detect silent mutations (i.e., not affecting amino acid sequence) are omitted.

In some chips, the probes from the first probe set are omitted corresponding to some or all positions of the reference sequences. Such chips comprise at least two probe sets. The first probe set has a plurality of probes. Each probe comprises a segment exactly complementary to a subsequence of a reference sequence except in at least one interrogation position. A second probe set has a corresponding probe for each probe in the first probe set.

The corresponding probe in the second probe set is identical to a sequence comprising the corresponding probe form the first probe set or a subsequence thereof that includes the at least one (and usually only one) interrogation position except that the at least one interrogation position is occupied by a different nucleotide in each of the two corresponding probes from the first and second probe sets. A third probe set, if present, also comprises a corresponding probe for each probe in the first probe set except at the at least one interrogation position, which differs in the corresponding probes from the three sets. Omission of probes having a segment exhibiting perfect complementarity to the reference sequence results in loss of control information, i.e., the detection of nucleotides in a target sequence that are the same As those in a reference sequence. However, similar information can be obtained by hybridizing a chip lacking probes from the first probe set to both target and reference sequences. The hybridization can be performed sequentially, or concurrently, if the target and reference are differentially labeled. In this situation, the presence of a mutation is detected by a shift in the background hybridization intensity of the reference sequence to a perfectly matched hybridization signal of the target sequence, rather than by a comparison of the hybridization intensities of probes from the first set with corresponding probes from the second, third and fourth sets.

Wildtype Probe Lane

When the chips comprise four probe sets, as discussed supra, and the probe sets are laid down in four lanes, an A-lane, a C-lane, a G-lane and a T or U-lane, the probe having a segment exhibiting perfect complementarity to a reference sequence varies between the four lanes from one column to another. This does not present any significant difficulty in computer analysis of the data from the chip. However, visual inspection of the hybridization pattern of the chip is sometimes facilitated by provision of an extra lane of probes, in which each probe has a segment exhibiting perfect complementarity to the reference sequence. This segment-is identical to a segment from one of the probes in the other four lanes (which lane depending on the column position). The extra lane of probes (designated the wildtype lane) hybridizes to a target sequence at all nucleotide positions except those in which deviations from the reference sequence occurs. The hybridization pattern of the wildtype lane thereby provides a simple visual indication of mutations.

Deletion, Insertion and Multiple-Mutation Probes

In some aspects, the chips provide an additional probe set specifically designed for analyzing deletion mutations. The additional probe set comprises a probe corresponding to each probe in the first probe set as described above. However, a probe from the additional probe set differs from the corresponding probe in the first probe set in that the nucleotide occupying the interrogation position is deleted in the probe from the additional probe set. Optionally, the probe from the additional probe set bears an additional nucleotide at one of its termini relative to the corresponding probe from the first probe set. The probe from the additional probe set will hybridize more strongly than the corresponding probe from the first probe set to a target sequence having a single base deletion at the nucleotide corresponding to the interrogation position. Additional probe sets are provided in which not only the interrogation position, but also an adjacent nucleotide is detected.

Similarly, other chips provide additional probe sets for analyzing insertions. For example, one additional probe set has a probe corresponding to each probe in the first probe set as described above. However, the probe in the additional probe set has an extra T nucleotide inserted adjacent to the interrogation position. Optionally, the probe has one fewer nucleotide at one of its termini relative to the corresponding probe from the first probe set. The probe from the additional probe set hybridizes more strongly than the corresponding probe from the first probe set to a target sequence having an A nucleotide inserted in a position adjacent to that corresponding to the interrogation position.

Similar additional probe sets are constructed having C, G or T/U nucleotides inserted adjacent to the interrogation position. Usually, four such probe sets, one for each nucleotide, are used in combination.

Other chips provide additional probes (multiple-mutation probes) for analyzing target sequences having multiple closely spaced mutations. A multiple-mutation probe is usually identical to a corresponding probe from the first set as described above, except in the base occupying the interrogation position, and except at one or more additional positions, corresponding to nucleotides in which substitution may occur in the reference sequence. The one or more additional positions in the multiple mutation probe are occupied by nucleotides complementary to the nucleotides occupying corresponding positions in the reference sequence when the possible substitutions have occurred.

Block Tiling

As noted in the discussion of the general tiling strategy, in one aspect, a probe in the first probe set can have more than one interrogation position. In this situation, a probe in the first probe set is sometimes matched with multiple groups of at least one, and usually, three additional probe sets. Three additional probe sets are used to allow detection of the three possible nucleotide substitutions at any one position. If only certain types of substitution are likely to occur (e.g., transitions), only one or two additional probe sets are required (analogous to the use of probes in the basic tiling strategy). To illustrate for the situation where a group comprises three additional probe sets, a first such group comprises second, third and fourth probe sets, each of which has a probe corresponding to each probe in the first probe set. The corresponding probes from the second, third and fourth probes sets differ from the corresponding probe in the first set at a first of the interrogation positions. Thus, the relative hybridization signals from corresponding probes from the first, second, third and fourth probe sets indicate the identity of the nucleotide in a target sequence corresponding to the first interrogation position. A second group of three probe sets (designated fifth, sixth and seventh probe sets), each also have a probe corresponding to each probe in the first probe set. These corresponding probes differ from that in the first probe set at a second interrogation position. The relative hybridization signals from corresponding probes from the first, fifth, sixth, and seventh probe sets indicate the identity of the nucleotide in the target sequence corresponding to the second interrogation position. As noted above, the probes in the first probe set often have seven or more interrogation positions. If there are seven interrogation positions, there are seven groups of three additional probe sets, each group of three probe sets serving to identify the nucleotide corresponding to one of the seven interrogation positions.

Each block of probes allows short regions of a target sequence to be read. For example, for a block of probes having seven interrogation positions, seven nucleotides in the target sequence can be read. Of course, a chip can contain any number of blocks depending on how many nucleotides of the target are of interest. The hybridization signals for each block can be analyzed independently of any other block. The block tiling strategy can also be combined with other tiling strategies, with different parts of the same reference sequence being tiled by different strategies.

The block tiling strategy offers two advantages over the basic strategy in which each probe in the first set has a single interrogation position. One advantage is that the same sequence information can be obtained from fewer probes. A second advantage is that each of the probes constituting a block (i.e., a probe from the first probe set and a corresponding probe from each of the other probe sets) can have identical 3′ and 5′ sequences, with the variation confined to a central segment containing the interrogation positions. The identity of 3′ sequence between different probes simplifies the strategy for solid phase synthesis of the probes on the chip and results in more uniform deposition of the different probes on the chip, thereby in turn increasing the uniformity of signal to noise ratio for different regions of the chip. A third advantage is that greater signal uniformity is achieved within a block.

Multiplex Tiling

In one aspect, in the block tiling strategy discussed above, the identity of a nucleotide in a target or reference sequence is determined by comparison of hybridization patterns of one probe having a segment showing a perfect match with that of other probes (usually three other probes) showing a single base mismatch. In multiplex tiling of the invention, the identity of at least two nucleotides in a reference or target sequence is determined by comparison of hybridization signal intensities of four probes, two of which have a segment showing perfect complementarity or a single base mismatch to the reference sequence, and two of which have a segment showing perfect complementarity or a double-base mismatch to a segment. The four probes whose hybridization patterns are to be compared each have a segment that is exactly complementary to a reference sequence except at two interrogation positions, in which the segment may or may not be complementary to the reference sequence. The interrogation positions correspond to the nucleotides in a reference or target sequence which are determined by the comparison of intensities. The nucleotides occupying the interrogation positions in the four probes are selected according to the following rule. The first interrogation position is occupied by a different nucleotide in each of the four probes. The second interrogation position is also occupied by a different nucleotide in each of the four probes. In two of the four probes, designated the first and second probes, the segment is exactly complementary to the reference sequence except at not more than one of the two interrogation positions. In other words, one of the interrogation positions is occupied by a nucleotide that is complementary to the corresponding nucleotide from the reference sequence and the other interrogation position may or may not be so occupied. In the other two of the four probes, designated the third and fourth probes, the segment is exactly complementary to the reference sequence except that both interrogation positions are occupied by nucleotides which are non-complementary to the respective corresponding nucleotides in the reference sequence.

There are number of ways of satisfying these conditions depending on whether the two nucleotides in the reference sequence corresponding to the two interrogation positions are the same or different. If these two nucleotides are different in the reference sequence (probability ¾), the conditions are satisfied by each of the two interrogation positions being occupied by the same nucleotide in any given probe. For example, in the first probe, the two interrogation positions would both be A, in the second probe, both would be C, in the third probe, each would be G, and in the fourth probe each would be T or U. If the two nucleotides in the reference sequence corresponding to the two interrogation positions are different, the conditions noted above are satisfied by each of the interrogation positions in any one of the four probes being occupied by complementary nucleotides. For example, in the first probe, the interrogation positions could be occupied by A and T, in the second probe by C and G, in the third probe by G and C and in the four probe, by T and A.

When the four probes are hybridized to a target that is the same as the reference sequence or differs from the reference sequence at one (but not both) of the interrogation positions, two of the four probes show a double-mismatch with the target and two probes show a single mismatch. The identity of probes showing these different degrees of mismatch can be determined from the different hybridization signals.

From the identity of the probes showing the different degrees of mismatch, the nucleotides occupying both of the interrogation positions in the target sequence can be deduced. For ease of illustration, the multiplex strategy has been initially described for the situation where there are two nucleotides of interest in a reference sequence and only four probes in an array. Of course, the strategy can be extended to analyze any number of nucleotides in a target sequence by using additional probes. In one variation, each pair of interrogation positions is read from a unique group of four probes. In a block variation, different groups of four probes exhibit the same segment of complementarity with the reference sequence, but the interrogation positions move within a block.

The block and standard multiplex tiling variants can of course be used in combination for different regions of a reference sequence. Either or both variants can also be used in combination with any of the other tiling strategies described.

Helper Mutations

Occasionally small regions of a reference sequence give a low hybridization signal as a result of annealing of probes. The self-annealing reduces the amount of probe effectively available for hybridizing to the target. Although such regions of the target are generally small and the reduction of hybridization signal is usually not so substantial as to obscure the sequence of this region, this concern can be avoided by the use of probes incorporating helper mutations.

The helper mutation(s) serve to break-up regions of internal complementarity within a probe and thereby prevent annealing. Usually, one or two helper mutations are quite sufficient for this purpose. The inclusion of helper mutations can be beneficial in any of the tiling strategies noted above. In general each probe having a particular interrogation position has the same helper mutation(s). Thus, such probes have a segment in common which shows perfect complementarity with a reference sequence, except that the segment contains at least one helper mutation (the same in each of the probes) and at least one interrogation position (different in all of the probes). For example, in the basic tiling strategy, a probe from the first probe set comprises a segment containing an interrogation position and showing perfect complementarity with a reference sequence except for one or two helper mutations. The corresponding probes from the second, third and fourth probe sets usually comprise the same segment (or sometimes a subsequence thereof including the helper mutation(s) and interrogation position), except that the base occupying the interrogation position varies in each probe.

Usually, the helper mutation tiling strategy is used in conjunction with one of the tiling strategies described above. The probes containing helper mutations are used to tile regions of a reference sequence otherwise giving low hybridization signal (e.g., because of self-complementarity), and the alternative tiling strategy is used to tile intervening regions.

Pooling Strategies

Pooling strategies of the invention can also employ arrays of immobilized probes. Probes can be immobilized in cells of an array, and the hybridization signal of each cell can be determined independently of any other cell. A particular cell may be occupied by pooled mixture of probes. Although the identity of each probe in the mixture is known, the individual probes in the pool are not separately addressable. Thus, the hybridization signal from a cell is the aggregate of that of the different probes occupying the cell. In general, a cell is scored as hybridizing to a target sequence if at least one probe occupying the cell comprises a segment exhibiting perfect complementarity to the target sequence.

A simple strategy to show the increased power of pooled strategies over a standard tiling is to create three cells each containing a pooled probe having a single pooled position, the pooled position being the same in each of the pooled probes. At the pooled position, there are two possible nucleotides, allowing the pooled probe to hybridize to two target sequences. In tiling terminology, the pooled position of each probe is an interrogation position. As will become apparent, comparison of the hybridization intensities of the pooled probes from the three cells reveals the identity of the nucleotide in the target sequence corresponding to the interrogation position (i.e., that is matched with the interrogation position when the target sequence and pooled probes are maximally aligned for complementarity).

The three cells are assigned probe pools that are perfectly complementary to the target except at the pooled position, which is occupied by a different pooled nucleotide in each probe. With 3 pooled probes, all 4 possible single base pair states (wild and 3 mutants) are detected. A pool hybridizes with a target if some probe contained within that pool is complementary to that target.

A cell containing a pair (or more) of oligonucleotides lights up when a target complementary to any of the oligonucleotide in the cell is present. Using the simple strategy, each of the four possible targets (wild and three mutants) yields a unique hybridization pattern among the three cells.

Since a different pattern of hybridizing pools is obtained for each possible nucleotide in the target sequence corresponding to the pooled interrogation position in the probes, the identity of the nucleotide can be determined from the hybridization pattern of the pools. Whereas, a standard tiling requires four cells to detect and identify the possible single-base substitutions at one location, this simple pooled 45 strategy only requires three cells.

In another aspect, pooling strategy for sequence analysis is the ‘Trellis’ strategy. In this strategy, each pooled probe has a segment of perfect complementarity to a reference sequence except at three pooled positions. One pooled position is an N pool. The three pooled positions may or may not be contiguous in a probe. The other two pooled positions are selected from the group of three pools consisting of (1) M or K, (2) R or Y and (3) W or S, where the single letters are IUPAC standard ambiguity codes. The sequence of a pooled probe is thus, of the form XXXN[(M/K) or (R/Y) or (W/S)][(M/K) or (R/Y) or (W/S)]XXXXX, where XXX represents bases complementary to the reference sequence. The three pooled positions may be in any order, and may be contiguous or separated by intervening nucleotides. For, the two positions occupied by [(M/K) or (R/Y) or (W/S)], two choices must be made. First, one must select one of the following three pairs of pooled nucleotides (1) M/K, (2) R/Y and (3) W/S. The one of three pooled nucleotides selected may be the same or different at the two pooled positions. Second, supposing, for example, one selects M/K at one position, one must then chose between M or K. This choice should result in selection of a pooled nucleotide comprising a nucleotide that complements the corresponding nucleotide in a reference sequence, when the probe and reference sequence are maximally aligned. The same principle governs the selection between R and Y, and between W and S. A trellis pool probe has one pooled position with four possibilities, and two pooled positions, each with two possibilities. Thus, a trellis pool probe comprises a mixture of 16 (4×2×2) probes. Since each pooled position includes one nucleotide that complements the corresponding nucleotide from the reference sequence, one of these 16 probes has a segment that is the exact complement of the reference sequence. A target sequence that is the same as the reference sequence (i.e., a wildtype target) gives a hybridization signal to each probe cell. Here, as in other tiling methods, the segment of complementarity should be sufficiently long to permit specific hybridization of a pooled probe to a reference sequence be detected relative to a variant of that reference sequence. Typically, the segment of complementarity is about 9-21 nucleotides.

A target sequence is analyzed by comparing hybridization intensities at three pooled probes, each having the structure described above. The segments complementary to the reference sequence present in the three pooled probes show some overlap. Sometimes the segments are identical (other than at the interrogation positions). However, this need not be the case.

For example, the segments can tile across a reference sequence in increments of one nucleotide (i.e., one pooled probe differs from the next by the acquisition of one nucleotide at the 5′ end and loss of a nucleotide at the 3′ end). The three interrogation positions may or may not occur at the same relative positions within each pooled probe (i.e., spacing from a probe terminus). All that is required is that one of the three interrogation positions from each of the three pooled probes aligns with the same nucleotide in the reference sequence, and that this interrogation position is occupied by a different pooled nucleotide in each of the three probes. In one of the three probes, the interrogation position is occupied by an N. In the other two pooled probes the interrogation position is occupied by one of (M/K) or (R/Y) or (W/S). In the simplest form of the trellis strategy, three pooled probes are used to analyze a single nucleotide in the reference sequence. Much greater economy of probes is achieved when more pooled probes are included in an array.

For example, consider an array of five pooled probes each having the general structure outlined above. Three of these pooled probes have an interrogation position that aligns with the same nucleotide in the reference sequence and are used to read that nucleotide. A different combination of three probes have an interrogation position that aligns with a different nucleotide in the reference sequence. Comparison of these three probe intensities allows analysis of this second nucleotide. Still another combination of three pooled probes from the set of five have an interrogation position that aligns with a third nucleotide in the reference sequence and these probes are used to analyze that nucleotide. Thus, three nucleotides in the reference sequence are fully analyzed from only five pooled probes. By comparison, the basic tiling strategy would require 12 probes for a similar analysis.

The trellis strategy can employ an array of probes having at least three cells, each of which is occupied by a pooled probe as described above. Consider the use of three such pooled probes for analyzing a target sequence, of which one position may contain any single base substitution to the reference sequence (i.e., there are four possible target sequences to be distinguished). Three cells are occupied by pooled probes having a pooled interrogation position corresponding to the position of possible substitution in the target sequence, one cell with an N′, one cell with one of M′ or K′, and one cell with R′ or Y′. An interrogation position corresponds to a nucleotide in the target sequence if it aligns adjacent with that nucleotide when the probe and target sequence are aligned to maximize 45 complementarity. Note that although each of the pooled probes has two other pooled positions, these positions are not relevant for the present illustration. The positions are only relevant when more than one position in the target sequence is to be read, a circumstance that will be considered later. For present purposes, the cell with the N′ in the interrogation position lights up for the wildtype sequence and any of the three single base substitutions of the target sequence.

A further class of strategies involving pooled probes are termed coding strategies. These strategies assign code words from some set of numbers to variants of a reference sequence. Any number of variants can be coded. The variants can include multiple closely spaced substitutions, deletions or insertions. The designation letters or other symbols assigned to each variant may be any arbitrary set of numbers, in any order. For example, a binary code is often used, but codes to other bases are entirely feasible. The numbers are often assigned such that each variant has a designation having at least one digit and at least one nonzero value for that digit.

For example, in a binary system, a variant assigned the number 101, has a designation of three digits, with one possible nonzero value for each digit. The designation of the variants are coded into an array of pooled probes comprising a pooled probe for each nonzero value of each digit in the numbers assigned to the variants. For example, if the variants are assigned successive number in a numbering system of base m, and the highest number assigned to a variant has n digits, the array would have about n×(m−1) pooled probes. In general, logₘ (3N+1) probes are required to analyze all variants of N locations in a reference sequence, each having three possible mutant substitutions. For example, 10 base pairs of sequence may be analyzed with only 5 pooled probes using a binary coding system. Each pooled probe has a segment exactly complementary to the reference sequence except that certain positions are pooled.

The segment should be sufficiently long to allow specific hybridization of the pooled probe to the reference sequence relative to a mutated form of the reference sequence. As in other tiling strategies, segments lengths of 9-21 nucleotides are typical. Often the probe has no nucleotides other than the 9-21 nucleotide segment. The pooled positions comprise nucleotides that allow the pooled probe to hybridize to every variant assigned a particular nonzero value in a particular digit. Usually, the pooled positions further comprises a nucleotide that allows the pooled probe to hybridize to the reference sequence. Thus, a wildtype target (or reference sequence) is immediately recognizable from all the pooled probes being lit.

When a target is hybridized to the pools, only those pools comprising a component probe having a segment that is exactly complementary to the target light up. The identity of the target is then decoded from the pattern of hybridizing pools. Each pool that lights up is correlated with a particular value in a particular digit. Thus, the aggregate hybridization patterns of each lighting pool reveal the value of each digit in the code defining the identity of the target hybridized to the array.

Bridging Strategy

Probes that contain partial matches to two separate (i.e., non contiguous) subsequences of a target sequence sometimes hybridize strongly to the target sequence. In certain instances, such probes have generated stronger signals than probes of the same length which are perfect matches to the target sequence. It is believed (but not necessary to the invention) that this observation results from interactions of a single target sequence with two or more probes simultaneously. This invention exploits this observation to provide arrays of probes having at least first and second segments, which are respectively complementary to first and second subsequences of a reference sequence. Optionally, the probes may have a third or more complementary segments. These probes can be employed in any of the strategies noted above.

The two segments of such a probe can be complementary to disjoint subsequences of the reference sequences or contiguous subsequences. If the latter, the two segments in the probe are inverted relative to the order of the complement of the reference sequence. The two subsequences of the reference sequence each typically comprises about 3 to 30 contiguous nucleotides. The subsequences of the reference sequence are sometimes separated by 0, 1, 2 or 3 bases. Often the sequences, are adjacent and nonoverlapping.

The bridging strategy can offer the following advantages:

(1) Higher discrimination between matched and mismatched probes, (2) The possibility of using longer probes in a bridging tiling, thereby increasing the specificity of the hybridization, without sacrificing discrimination, (3) The use of probes in which an interrogation position is located very off-center relative to the regions of target complementarity. This may be of particular advantage when, for example, when a probe centered about one region of the target gives low hybridization signal. The low signal is overcome by using a probe centered about an adjoining region giving a higher hybridization signal. (4) Disruption of secondary structure that might result in annealing of certain probes (see previous discussion of helper mutations).

Deletion Tiling

The invention also provides a deletion tiling strategy. Deletion tiling is related to both the bridging and helper mutant strategies described above. In the deletion strategy, comparisons are performed between probes sharing a common deletion but differing from each other at an interrogation position located outside the deletion. For example, a first probe comprises first and second segments, each exactly complementary to respective first and second subsequences of a reference sequence, wherein the first and second subsequences of the reference sequence are separated by a short distance (e.g., 1 or 2 nucleotides). The order of the first and second segments in the probe is usually the same as that of the complement to the first and second subsequences in the reference sequence.

Such tilings sometimes offer superior discrimination in hybridization intensities between the probe having an interrogation position complementary to the target and other probes. Thermodynamically, the difference between the hybridizations to matched and mismatched targets for the probe set shown above is the difference between a single-base bulge, and a large asymmetric loop (e.g., two bases of target, one of probe). This often results in a larger difference in stability than the comparison of a perfectly matched probe with a probe showing a single base mismatch in the basic tiling strategy.

The use of deletion or bridging probes is quite general. These probes can be used in any of the tiling strategies of the invention. As well as offering superior discrimination, the use of deletion or bridging strategies is advantageous for certain probes to avoid self-hybridization (either within a probe or between two probes of the same sequence).

Preparation of Target Samples

The target polynucleotide, whose sequence is to be determined, is usually isolated from a tissue sample. If the target is genomic, the sample may be from any tissue (except exclusively red blood cells). For example, whole blood, peripheral blood lymphocytes or PBMC, skin, hair or semen are convenient sources of clinical samples. These sources are also suitable if the target is RNA. Blood and other body fluids are also a convenient source for isolating viral nucleic acids. If the target is mRNA, the sample is obtained from a tissue in which the mRNA is expressed. If the polynucleotide in the sample is RNA, it is usually reverse transcribed to DNA. DNA samples or cDNA resulting from reverse transcription are usually amplified, e.g., by PCR. Depending on the selection of primers and amplifying enzyme(s), the amplification product can be RNA or DNA.

Paired primers are selected to flank the borders of a target polynucleotide of interest. More than one target can be simultaneously amplified by multiplex PCR in which multiple paired primers are employed. The target can be labeled at one or more nucleotides during or after amplification. For some target polynucleotides (depending on size of sample), e.g., episomal DNA, sufficient DNA is present in the tissue sample to dispense with the amplification step.

When the target strand is prepared in single-stranded form as in preparation of target RNA, the sense of the strand should of course be complementary to that of the probes on the chip. This is achieved by appropriate selection of primers. The target can be fragmented before application to the chip to reduce or eliminate the formation of secondary structures in the target. The average size of targets segments following hybridization is usually larger than the size of probe on the chip.

Sequencing

This invention provides a method of performing whole cell engineering that comprises the step of cell screening. In one aspect, the step of cell screening may comprise the step of genomic sequencing. In one exemplification, genome sequencing can be accomplished according to the enzymatic/Sanger method (described in F. Sanger, S. Nicklen, and A. R. Coulson, Proc. Nati. Acad. Sci, USA, 74:5463-5467 (1977)) and involve cloning and subcloning (described in U.S. Pat. No. 4725677; Chen and Seeburg, DNA 4, 165-170 (1985); Lim et al., Gene Anal., Techn. 5, 32-39 (1988); PCR Protocols—A Guide to Methods and Applications. Innis et al., editors, Academic Press, San Diego (1990); Innis et al., Proc. Nat. Acad. Sci. USA 85, 9436-9440 (1988)).

In another exemplification, sequencing can be accomplished according to the chemical/Maxam and Gilbert method which is described in references: A. M. Maxam, and W. Gilbert, Proc. Nat. Acad. of Sci., USA, 74:560-564 (1977) and Church et al., Proc. Natl. Acad. Sci., 81:1991 (1984). In additional exemplifications, genome sequencing can be accomplished by methodology described by Guo and Wu (Guo and Wu, Nucleic Acids Res., 10:2065 (1982); and Meth. Enz.,100:60 (1983)) or those methods that utilize 3′hydroxy-protected and labeled nucleotides as exemplified in the following references: Churchich, J. E., Eur. J. Biochem., 231:736 (1995); Metzket, M. L. et al.,Nucleic Acids Research, 22:4259 (1994); Beabealashvilli, R. S. et al, Biochimica et Biophysica Acta, 868:136 (1986); Chidgeavadze, Z. G.; Kukhanova, M. K. et al. Biochimica et Biophysica Acta, 868:145 (1986); Hiratsuka, T et Biophysica Acta, 742:496 (1983); Jeng, S. J. and Guillory, R. J. J., Supramolecular Structure, 3:448 (1975).

The invention also provides that sequencing may be read by autoradiography using radioisotopes (as described in Ornstein et al., Biotechniques 2, 476 (1985)) or by using non-radioactively labeling strategies that have been integrated into partly automated DNA sequencing procedures (Smith et al., Nature M, 674-679 (1986) and EPO Pat. No. 873 00998.9; Du Pont De Nemours EPO Application No. 03 59225; Ansorge et al., L Biochem. Biophys. Method 13, 325-32 (19860; Prober et al. Science M, 336-41 (1987); Applied Biosystems, PCT Application WO 91/05060; Smith et al., Science 235, G89 (1987); U.S. Pat. Nos. 570,973 and 689,013), Du Pont De Nemours, U.S. Pat. Nos. 881372 and 57566, Ansorge et al. Nucleic Acids Res. 15-, 4593-4602 (1987) and EMBL Pat. Application DE P3724442 and P3805808.1) and Hitachi (JP 1-90844 and DE 4011991 A1; U.S. Pat. No. 4,729,947; PCT Application W092/02635; U.S. Pat. No. 594676; Beck, O'Keefe, Coull and Köster, Nucleic Acids Res. 7, 5115-5123 (1989). L7 and Beck and Köster, Anal. Chem. 62 2258-2270 (1990); Church et al., Science 240, 185-188 (1988); Köster et al., Nucleic Acids Res. Symposium Ser. No. 24, 318-321 (1991), University of Utah, PCT Application No. WO 90/15883; Smith et al., Nature (1986) 321:674-679; Orion-Yhtyma Oy, U.S. Pat. No. 277,643; M. Uhlen et al. Nucleic Acids Res. 16, 3025-38 (1988); Cemu Bioteknik, PCT Application No. WO 89/09282 and Medical Research Council, GB, PCT Application No. WO 92/03575; Du Pont De Nemours, PCT Application WO 91/11533).

In addition, this invention provides for various methods of reading sequencing data such as capillary zone electrophoresis (described in Jorgenson et al., J. Chromatography 352, 337 (1986); Gesteland et al., Nucleic Acids Res. 18, 1415-1419 (1990)), mass spectrometry (including ES [described in Fenn et al. J. Phys. Chem. 18, 4451-59 (1984); PCT Application No. WO 90/14148; R. D. Smith et al., Anal. Chem. 62, 882-89 (1990) and B. Ardrey, Electrospray Mass Spectrometry, Spectroscopy Europe 4, 10-18 (1992)] and MALDI [Hillenkamp et al. Matrix Assisted UV-Laser Desorption/Ionization: A New Approach to Mass Spectrometry of Large Biomolecules, Biological Mass Spectrometry (Burlingame and McCloskey, editors), Elsevier Science Publishers, Amsterdam, pp. 49-60, (1990); Williams et al., Science, 246, 1585-87 (1989); Williams et al., Rapid Communications in Mass Spectrometry, 4, 348-351 (1990)]), tube gel electrophoresis and a mass analyzer to sequence (described in EPO Patent Applications No. 0360676 A1 and 0360677). In order to analyze the sequencing data, this invention provides for the use of probes in large arrays (as described in PCT patent Publication No. 92/10588; U.S. Pat. No. 5,143,854; U.S. application Ser. No. 07/805,727; U.S. Pat. No. 5,202,231; PCT patent Publication No. 89/10977).

The invention provides a method of performing whole cell engineering comprising the step of cell screening. In one aspect, the method includes DNA amplification. DNA can be amplified by a variety of procedures including cloning (Sambrook et at., Molecular Cloning: A Laboratory Manual., Cold Spring Harbor Laboratory Press, 1989), polymerase chain reaction (PCR) (C. R. Newton and A. Graham, PCF, BIOS Publishers, 1994; Bevan et al., “Sequencing of PCR-Amplified DNA” PCR Meth. App. 4:222 (1992)), ligase chain reaction (LCR) (F. Barany Proc. Natl. Acad Sci USA 88, 189-93 (1991), strand displacement amplification (SDA) (G. Terrance Walker et al., Nucleic Acids Res. 22, 2670-77 (1994)) and variations such as RT-PCR (Arens, M. Clin Microbiol Rev, 12(4):612-26 (1999)), allele-specific amplification (ASA) (Nichols, W. C. et al. Genomics. October ;5(3):535-40(1989); Giffard, P. M. et al. Anal Biochem,;292(2):207-15 (2001)).

In additional aspects of this invention, it provides for additional sequencing methods (as described in Labeit et al., MA 5, 173-177 (1986); Amersham, PCT-Application GB86/00349; Eckstein et al., Nucleic Acids Res. 1˜, 9947 (1988); Max-Planck-Geselischaft, DE 3930312 A1; Saiki, R. et al., Science 239:487-491 (1998); Sarkat, G. and Bolander Mark E., Semi Exponential Cycle Sequencing Nucleic Acids Research, 1995, Vol. 23, No. 7, p. 1269-1270).

This invention also provides for the following sequencing strategies: shotgun sequencing, transposon-mediated directed sequencing (Strathmann, M. et al. Proc Natl Acad Sci USA (1991) 88:1247-1250), and large scale variations thereof (as exemplified in K. B. Mullis et al., U.S. Pat. Nos. 4,683,202; 7/1987; 435/91; and 4,683,195, 7/1987; 435/6).

In alternative aspects, the step of genomic sequencing includes constructing ordered clone maps of DNA sequencing (as described in sections of U.S. Patent Publication No. 5604100 and PCT Pat. Publication No. WO9627025). This invention provides that the method of genome sequencing be achieved by various steps that may utilize modifications of certain methods mentioned above (described in the following patents: PCT Publication Nos. WO9737041, WO9742348, WO9627025, WO9831834, WO9500530, and WO9831833; US Pat. Publication Nos. U.S. Pat. No. 5,604,100, U.S. Pat. No. 5,670,321, U.S. Pat. No. 5,453,247, U.S. Pat. No. 5,994,058, and U.S. Pat. No. 5,354,656).

Annotating

In one aspect this invention provides for the use of a relational database system for storing and manipulating biomolecular sequence information and storing and displaying genetic information, the database including genomic libraries for a plurality of types of organisms, the libraries having multiple genomic sequences, at least some of which represent open reading frames located along a contiguous sequence on each the plurality of organisms' genomes, and a user interface capable of receiving a selection of two or more of the genomic libraries for comparison and displaying the results of the comparison. Associated with the database is a software system that allows a user to determine the relative position of a selected gene sequence within a genome. The system allows execution of a method of displaying the genetic locus of a biomolecular sequence. The method involves providing a database including multiple biomolecular sequences, at least some of which represent open reading frames located along a contiguous sequence on an organism's genome. The system also provides a user interface capable of receiving a selection of one or more probe open reading frames for use in determining homologous matches between such probe open reading frame(s) and the open reading frames in the genomic libraries, and displaying the results of the determination. An open reading frame for the sequence is selected and displayed together with adjacent open reading frames located upstream and downstream in the relative positions in which they occur on the contiguous sequence.

In one aspect, the invention provides a relational database system for storing biomolecular sequence information in a manner that allows sequences to be catalogued and searched according to one or more protein function hierarchies. The hierarchies allow searches for sequences based upon a protein's biological function or molecular function. Also disclosed is a mechanism for automatically grouping new sequences into protein function hierarchies. This mechanism uses descriptive information obtained from “external hits” which are matches of stored sequences against gene sequences stored in an external database such as GenBank. The descriptive information provided with the external database is evaluated according to a specific algorithm and used to automatically group the external hits (or the sequences associated with the hits) in the categories. Ultimately, the biomolecular sequences stored in databases of this invention are provided with both descriptive information from the external hit and category information from a relevant hierarchy or hierarchies.

Disclosed is a relational database system for storing biomolecular sequence information in a manner that allows sequences to be catalogued and searched according to association with one or more projects for obtaining full-length biomolecular sequences from shorter sequences. The relational database has sequence records containing information identifying one or more projects to which each of the sequence records belong. Each project groups together one or more biomolecular sequences generated during work to obtain a full-length gene sequence from a shorter sequence. The computer system has a user interface allowing a user to selectively view information regarding one or more projects. The relational database also provides interfaces and methods for accessing and manipulating and analyzing project-based information.

Polymer sequences can be assembled into bins. A first number of bins are populated with polymer sequences. The polymer sequences in each bin are assembled into one or more consensus sequences representative of the polymer sequences of the bin. The consensus sequences of the bins are compared to determine relationships, if any, between the consensus sequences of the bins. The bins are modified based on the relationships between the consensus sequences of the bins. The polymer sequences are reassembled in the modified bins to generate one or more modified consensus sequences for each bin representative of the modified bins. In another aspect of the invention, sequence similarities and dissimilarities are analyzed in a set of polymer sequences. Pairwise alignment data is generated for pairs of the polymer sequences. The pairwise alignment data defines regions of similarity between the pairs of polymer sequences with boundaries. Additional boundaries in particular polymer sequences are determined by applying at least one boundary from at least one pairwise alignment for one pair of polymer sequences to at least one other pairwise alignment for another pair of polymer sequences including one of the particular polymer sequences. Additional regions of similarity are generated based on the boundaries.

### ANNOTATING—GENERAL METHODOLOGY

In one aspect the invention provides relational databases for storing and retrieving biological information. More particularly the invention relates to systems and methods for providing sequences of biological molecules in a relational format allowing retrieval in a client-server environment and for providing full-length cDNA sequences in a relational format allowing retrieval in a client-server environment.

### ANNOTATING—EXEMPLARY ASPECTS

The annotation methods of this invention include those described in PCT patent publication Nos. 98/26407, 98/26408, and 99/49403 and U.S. Pat. Nos. 6,023,659 and 5,953,727. Thus, in one aspect, this present invention provides relational database systems for storing and analyzing biomolecular sequence information together with biological annotations detailing the source and interpretation the sequence data. The present invention provides a powerful database tool for drug development and other research and development purposes.

The present invention provides relational database systems for storing and analyzing biomolecular sequence information together with biological detailing the source and interpretation the sequence data. Disclosed is a relational database systems for storing and displaying genetic information.

Associated with the database is a software system the allows a user to determine the relative position of a selected gene sequence within a genome. The system allows execution of a method of displaying the genetic locus of a biomolecular sequence. The method involves providing a database including multiple biomolecular sequences, at least some of which represent open reading frames located along a contiguous sequence on an organism's genome. An open reading frame for the sequence is selected and displayed together with adjacent open reading frames located upstream and downstream in the relative positions in which they occur on the contiguous sequence.

The invention provides a method of displaying the genetic locus of a biomolecular sequence. The method involve providing a database including multiple biomolecular sequences, at least some of which represent open reading frames located along a contiguous sequence on an organism's genome. The method further involves identifying a selected open reading frame, and displaying the selected open reading frame together with adjacent open reading frames located upstream and downstream from the selected open reading frame.

The adjacent open reading frames and the selected open reading frame are displayed in the relative positions in which they occur on the contiguous sequence, textually and/or graphically. The method of the invention may be practiced with sequences from microbial organisms, and the sequences may include nucleic acid or protein sequences.

The invention also provides a computer system including a database having multiple biomolecular sequences, at least some of which represent open reading frames located along a contiguous sequence on an organism's genome.

The computer system also includes a user interface capable of identifying a selected open reading frame, and displaying the selected open reading frame together with adjacent open reading frames located upstream and downstream from the selected open reading frame. The adjacent the open reading frames and the selected open reading frame are displayed in the relative positions in which they occur on the contiguous sequence. The user interface may also capable of detecting a scrolling command, and based upon the direction and magnitude of the scrolling command, identifying a new selected open reading frame from the contiguous sequence.

The invention further provides a computer program product comprising a computer-usable medium having computer-readable program code embodied thereon relating to a database including multiple biomolecular sequences, at least some of which represent open reading frames located along a contiguous sequence on an organism's genome. The computer program product includes computer-readable program code for identifying a selected open reading frame, and displaying the selected open reading frame together with adjacent open reading frames located upstream and downstream from the selected open reading frame. The adjacent open reading frames and the selected open reading frame are displayed in the relative positions in which they occur on the contiguous sequence.

Comparative Genomics is a feature of the database system of the present invention which allows a user to compare the sequence data of sets of different organism types. Comparative searches may be formulated in a number of ways using the Comparative Genomics feature. For example, genes common to a set of organisms may be identified through a “commonality” query, and genes unique to one of a set of organisms may be identified through a “subtraction” query.

Electronic Southern is a feature of the present database system which is useful for identifying genomic libraries in which a given gene or ORF exists. A Southern analysis is a conventional molecular biology technique in which a nucleic acid of known sequence is used to identify matching (complementary) sequences in a sample of nucleic acid to be analyzed. Like their laboratory counterparts, Electronic Southerns according to the present invention may be used to locate homologous matches between a “probe” DNA sequence and a large number of DNA sequences in one or more libraries.

The present invention provides a method of comparing genetic complements of different types of organisms. The method involves providing a database having sequence libraries with multiple biomolecular sequences for different types of organisms, where at least some of the sequences represent open reading frames located along one or more contiguous sequences on each of the organisms' genomes. The method further involves receiving a selection of two or more of the sequence libraries for comparison, determining open reading frames common or unique to the selected sequence libraries, and displaying the results of the determination.

The invention also provides a method of comparing genomic complements of different types of organisms. The method involves providing a database having genomic sequence libraries with multiple biomolecular sequences for different types of organisms, where at least some of the sequences represent open reading frames located along one or more contiguous sequences on each of the organisms' genomes. The method further involves receiving a selection of two or more of the sequence libraries for comparison, determining sequences common or unique to the selected sequence libraries, and displaying the results of the determination.

The invention further provides a computer system including a database containing genomic libraries for different types of organisms, which libraries have multiple genomic sequences, at least some of which representing open reading frames located along one or more contiguous sequences on each the organisms' genomes. The system also includes a user interface capable of receiving a selection of two or more genomic libraries for comparison and displaying the results of the comparison.

Another aspect of the present invention provides a method of identifying libraries in which a given gene exists. The method involves providing a database including genomic libraries for one or more types of organisms. The libraries have multiple genomic sequences, at least some of which represent open reading frames located along one or more contiguous sequences on each the organisms' genomes. The method further involves receiving a selection of one or more probe sequences, determining homologous matches between the selected probe sequences and the sequences in the genomic libraries, and displaying the results of the determination.

The invention also provides a computer system including a database including genomic libraries for one or more types of organisms, which libraries have multiple genomic sequences, at least some of which represent open reading frames located along one or more contiguous sequences on each the organisms' genomes. The system also includes a user interface capable of receiving a selection of one or more probe sequences for use in determining homologous matches between one or more probe sequences and the sequences in the genomic libraries, and displaying the results of the determination.

Also provided is a computer program product including a computer-usable medium having computer-readable program code embodied thereon relating to a database including genomic libraries for one or more types of organisms. The libraries have multiple genomic sequences, at least some of which represent open reading frames located along one or more contiguous sequences on each the organisms' genomes. The computer program product includes computer-readable program code for providing, within a computing system, an interface for receiving a selection of two or more genomic libraries for comparison, determining sequences common or unique to the selected genomic libraries, and displaying the results of the determination.

Additionally provided is a computer program product including a computer-usable medium having computer-readable program code embodied thereon relating to a database including genomic libraries for one or more types of organisms. The libraries have multiple genomic sequences, at least some of which represent open reading frames located along one or more contiguous sequences on each the organisms' genomes. The computer program product includes computer-readable program code for providing, within a computing system, an interface for receiving a selection of one or more probe open reading frames, determining homologous matches between the probe sequences and the sequences in the genomic libraries, and displaying the results of the determination.

The invention further provides a method of presenting the genetic complement of an organism. The method involves providing a database including sequence libraries for a plurality of types of organisms, where the libraries have multiple biomolecular sequences, at least some of which represent open reading frames located along one or more contiguous sequences on each of the organisms' genomes. The method further involves receiving a selection of one of the sequence libraries, determining open reading frames within the selected sequence library, and displaying the results as one or more unique identifiers for groups of related opening reading frames.

The present invention provides relational database systems for storing biomolecular sequence information in a manner that allows sequences to be catalogued and searched according to one or more protein function hierarchies. The hierarchies are provided to allow carefully tailored searches for sequences based upon a protein's biological function or molecular function. To make this capability available in large sequence databases, the invention provides a mechanism for automatically grouping new sequences into protein function hierarchies. This mechanism takes advantage of descriptive information obtained from “external hits” which are matches of stored sequences against gene sequences stored in an external database such as GenBank. The descriptive information provided with GenBank is evaluated according to a specific algorithm and used to automatically group the external hits (or the sequences associated with the hits) in the categories. Ultimately, the biomolecular sequences stored in databases of this invention are provided with both descriptive information from the external hit and category information from a relevant hierarchy or hierarchies.

The invention provides a computer system having a database containing records pertaining to a plurality of biomolecular sequences. At least some of the biomolecular sequences are grouped into a first hierarchy of protein function categories, the protein function categories specifying biological functions of proteins corresponding to the biomolecular sequences and the first hierarchy. The hierarchy includes a first set of protein function categories specifying biological functions at a cellular level, and a second set of protein function categories specifying biological functions at a level above the cellular level. The computer system of the invention also includes a user interface allowing a user to selectively view information regarding the plurality of biomolecular sequences as it relates to the first hierarchy. The computer system may also include additional protein function categories based, for example, on molecular or enzymatic function of proteins. The biomolecular sequences may include nucleic acid or amino acid sequences. Some of said biomolecular sequences may be provided as part of one or more projects for obtaining full-length gene sequences from shorter sequences, and the database records may contain information about such projects.

The invention also provides a method of using a computer system to present information pertaining to a plurality of biomolecular sequence records stored in a database. The method involves displaying a list of the records or a field for entering information identifying one or more of the records, identifying one or more of the records that a user has selected from the list or field, matching the one or more selected records with one or more protein function categories from a first hierarchy of protein function categories into which at least some of the biomolecular sequence records are grouped, and displaying the one or more categories matching the one or more selected records. The protein function categories specify biological functions of proteins corresponding to the biomolecular sequences and the first hierarchy includes a first set of protein function categories specifying biological functions at a cellular level, and a second set of protein function categories specifying biological functions at a tissue level. The method may also involve matching the records against other protein function hierarchies, such as hierarchies based on molecular and/or enzymatic function, and displaying the results. At least some of the biomolecular sequences may be provided as part of one or more projects for obtaining full-length gene sequences from shorter sequences, and the database records may contain information about those projects.

Additionally, the invention provides a method of using a computer system to present information pertaining to a plurality of biomolecular sequence records stored in a database. The method involves displaying a list of one or more protein biological function categories from a first hierarchy of protein biological function categories into which at least some of the biomolecular sequence records are grouped, identifying one or more of the protein biological function categories that a user has selected from the list, matching the one or more selected protein biological function categories with one or more biomolecular sequence records which are grouped in the selected protein biological function categories, and displaying the one or more sequence records matching the one or more selected protein biological function categories. The protein biological function categories specify biological functions of proteins corresponding to the biomolecular sequences and the first hierarchy includes a first set of protein biological function categories specifying biological functions at a cellular level, and a second set of protein biological function categories specifying biological functions at a tissue level. The method may also involve matching the records against other protein function hierarchies, such as hierarchies based on molecular and/or enzymatic function, and displaying the results. At least some of the biomolecular sequences may be provided as part of one or more projects for obtaining full-length gene sequences from shorter sequences, and the database records may contain information about those projects.

Another aspect of the invention provides a database system having a plurality of internal records. The database includes a plurality of sequence records specifying biomolecular sequences, at least some of which records reference hits to an external database, which hits specify genes having sequences that at least partially match those of the biomolecular sequences. The database also includes a plurality of external hit records specifying the hits to the external database, and at least some of the records reference protein function hierarchy categories which specify at least one of biological functions of proteins or molecular functions of proteins. At least some of the biomolecular sequences may be provided as part of one or more projects for obtaining full-length gene sequences from shorter sequences, and the database records may contain information about those projects.

Further aspects of the present invention provide a method of using a computer system and a computer readable medium having program instructions to automatically categorize biomolecular sequence records into protein function categories in an internal database. The method and program involve receiving descriptive information about a biomolecular sequence in the internal database from a record in an external database pertaining to a gene having a sequence that at least partially matches that of the biomolecular sequence. Next, a determination is made whether the descriptive information contains one or more terms matching one or more keywords associated with a first protein function category, the keywords being terms consistent with a classification in the first protein function category. When at least one keyword is found to match a term in the descriptive information, a determination is made whether the descriptive information contains a term matching one or more anti-keywords associated with the first protein function category, the anti-keywords being terms inconsistent with a classification in the first protein function category. Then, the biomolecular sequence is grouped in the first protein function category when the descriptive information contains a term matching a keyword but contains no term matching an anti-keyword.

The present invention provides relational database systems for storing biomolecular sequence information in a manner that allows sequences to be catalogued and searched according to one or more characteristics. The sequence information of the database is generated by one or more “projects” which are concerned with identifying the full-length coding sequence of a gene (i.e., mRNA). The projects involve the extension of an initial sequenced portion of a clone of a gene of interest (e.g., an EST) by a variety of methods which use conventional molecular biological techniques, recently developed adaptations of these techniques, and certain novel database applications. Data accumulated in these projects may be provided to the database of the present invention throughout the course of the projects and may be available to database users (subscribers) throughout the course of these projects for research, product (i.e., drug) development, and other purposes.

In one aspect, the database of the present invention and its associated projects may provide sequence and related data in amounts and forms not previously available. The present invention can make partial and full-length sequence information for a given gene available to a user both during the course of the data acquisition and once the full-length sequence of the gene has been elucidated. The database can provide a variety of tools for analysis and manipulation of the data, including Northern analysis and Expression summaries. The present invention should permit more complete and accurate annotation of sequence data, as well as the study of relationships between genes of different tissues, systems or organisms, and ultimately detailed expression studies of full-length gene sequences.

The invention provides a computer system including a database having sequence records containing information identifying one or more projects to which each of the sequence records belong. Each project groups together one or more biomolecular sequences generated during work to obtain a full-length gene sequence from a shorter sequence. The computer system also has a user interface allowing a user to selectively view information regarding one or more projects. The biomolecular sequences may include nucleic acid or amino acid sequences. The user interface may allow users to view at least three levels of project information including a project information results level listing at least some of the projects in said database, a sequence information results level listing at least some of the sequences associated with a given project, and a sequence retrieval results level sequentially listing monomers which comprise a given sequence.

A method of using a computer system and a computer program product to present information pertaining to a plurality of sequence records stored in a database are also provided by the present invention. The sequence records contain information identifying one or more projects to which each of the sequence records belong. Each of the projects groups one or more biomolecular sequences generated during work to obtain a full-length gene sequence from a shorter sequence. The method and program involve providing an interface for entering query information relating to one or more projects, locating data corresponding to the entered query information, and displaying the data corresponding to the entered query information.

Additionally, the invention provides a method of using a computer system to present information pertaining to a plurality of sequence records stored in a database. The sequence records contains information identifying one or more projects to which each of the sequence records belong. Each of the projects groups one or more biomolecular sequences generated during work to obtain a full-length gene sequence from a shorter sequence. The method involves displaying a list of one or more project identifiers, determining which project identifier or identifiers from the list is selected by a user, then displaying a second list of one or more biomolecular sequence identifiers associated with the selected project identifier or identifiers, determining which sequence identifier or identifiers from the second list has been selected by a user, and displaying a third list of one or more sequences corresponding to the selected sequence identifier or identifiers. Following the display of the third list, a determination may be made whether and which sequence from the third list has been selected by a user. If a sequence is selected, a sequence alignment search of the selected sequence against other data-based sequences may be initiated, and the results of the alignment search displayed.

For Electronic Northern analysis, the invention further provides a computer system including a database having sequence records containing information identifying one or more projects to which each of the sequence records belong, each of said projects grouping one or more biomolecular sequences generated during work to obtain a full-length gene sequence from a shorter sequence. The system also has a user interface capable of allowing a user to select one or more project identifiers or project member identifiers specifying one or more sequences to be compared with one or more cDNA sequence libraries, and displaying matches resulting from that comparison.

A method of using a computer system to present comparative information pertaining to a plurality of sequence records stored in a database is also provided by the present invention. The sequence records contain information identifying one or more projects to which each of the sequence records belong, each of the projects grouping one or more biomolecular sequences generated during work to obtain a full-length gene sequence from a shorter sequence. The method involves providing an interface capable of allowing a user to select one or more project identifiers or project member identifiers specifying one or more sequences, comparing the one or more specified sequences with one or more cDNA sequence libraries, and displaying matches resulting from the comparison.

In addition, for Expression analysis, the invention provides a computer system including a database having sequence records containing information identifying one or more projects to which each of the sequence records belong, each of the projects grouping one or more biomolecular sequences generated during work to obtain a full-length gene sequence from a shorter sequence. The system also has a user interface allowing a user to view expression information pertaining to the projects by selecting one or more expression categories for a query, and displaying the result of the query.

A method of using a computer system to view expression information pertaining to one or more projects, each of the projects grouping one or more biomolecular sequences generated during work to obtain a full-length gene sequence from a shorter sequence, is also provided by the invention. The computer system includes a database storing a plurality of sequence records, the sequence records containing information identifying one or more projects to which each of the sequence records belong. The method involves providing an interface which allows a user to select one or more expression categories as a query, locating projects belonging to the selected one or more expression categories, and displaying a list of located projects.

The present invention provides a computer system including a database having sequence records containing information identifying one or more projects to which each of the sequence records belong, each of the projects grouping one or more biomolecular sequences generated during work to obtain a full-length gene sequence from a shorter sequence. This computer system has a user interface allowing a user to selectively view information regarding said one or more projects and which displays information to a user in a format common to one or more other sequence databases.

Polymer sequences are assembled into bins. A first number of bins are populated with polymer sequences. The polymer sequences in each bin are assembled into one or more consensus sequences representative of the polymer sequences of the bin. The consensus sequences of the bins are compared to determine relationships, if any, between the consensus sequences. The bins are modified based on the relationships between the consensus sequences. The polymer sequences are reassembled in the modified bins to generate one or more modified consensus sequences for each bin representative of the modified bins.

In another aspect of the invention, sequence similarities and dissimilarities are analyzed in a set of polymer sequences. Pairwise alignment data is generated for pairs of the polymer sequences. The pairwise alignment data defines regions of similarity between the pairs of polymer sequences with boundaries. Additional boundaries in particular polymer sequences are determined by applying at least one boundary from at least one pairwise alignment for one pair of polymer sequences to at least one other pairwise alignment for another pair of polymer sequences including one of the particular polymer sequences. Additional regions of similarity are generated based on the boundaries.

### ANNOTATING—RELATIONAL DATABASES

The present invention provides an improved relational database for storing and manipulating genomic sequence information. While the invention is described in terms of a database optimized for microbial data, it is by no means so limited. The invention may be employed to investigate data from various sources. For example, the invention covers databases optimized for other sources of sequence data, such as animal sequences (e.g., human, primate, rodent, amphibian, insect, etc.), plant sequences and microbial sequences. In the following description, numerous specific details are set forth in order to provide a thorough understanding of the present invention. It will be apparent, however, that the present invention may be practiced without limitation to some of the specific details presented herein.

Generally, the present invention provides an improved relational database for storing sequence information. The invention may be employed to investigate data from various sources. For example, it may catalogue animal sequences (e.g., human, primate, rodent, amphibian, insect, etc.), plant sequences, and microbial sequences.

Transcriptome Analysis or RNA Profiling

The characterization of RNA expression and transcript populations (the transcriptome) can be referred to as RNA profiling and/or expression profiling, utilizing high throughput techniques such as RNA differential displays and DNA microarrays. One potential method to characterize gene expression, SAGE (Serial Analysis of Gene Expression) utilizes combinatorial chemistry technology and short sequence tags in the screening of compound libraries. For further information see references: Burge, C. B. 2001. Chipping away at the transcriptome. Nat Genet, 27(3): 232-4; Hughes, T. R. and Shoemaker, D. D. 2001. DNA microarrays for expression profiling. Curr Opin Chem Biol, 5(1): 21-5; Yamamoto, M. et al. 2001. Use of serial analysis of gene expression (SAGE) technology. J Immunol Methods 250(1-2):45-66.

Screening and Selecting Nucleotides for Protein Binding

One aspect of the invention provides for screening methods that include the user of recombinant and in vitro chemical synthesis methods. In these hybrid methods, cell-free enzymatic machinery is employed to accomplish the in vitro synthesis of the library members (i.e., peptides or polynucleotides). In one type of method, RNA molecules with the ability to bind a predetermined protein or a predetermined dye molecule were selected by alternate rounds of selection and PCR amplification (Tuerk and Gold, 1990; Ellington and Szostak, 1990). A similar technique was used to identify DNA sequences which bind a predetermined human transcription factor (Thiesen and Bach, 1990; Beaudry and Joyce, 1992; PCT patent publications WO 92/05258 and WO 92/14843).

Proteomics

In another aspect of this invention, this invention relates to the emerging field of proteomics. Proteomics involves the qualitative and quantitative measurement of gene activity by detecting and quantitating expression at the protein level, rather than at the messenger RNA level. Proteomics also involves the study of non-genome encoded events, including the post-translational modification of proteins (including glycosylation or other modifications), interactions between proteins, and the location of proteins within a cell. The structure, function, and/or level of activity of the proteins expressed by the cell are also of interest. Essentially, proteomics involves the study of part or all of the status of the total protein contained within or secreted by a cell. Proteomics requires means of separating proteins in complex mixtures and identifying both low-and high-abundance species. Examples of powerful methods currently used to resolve complex protein mixtures are 2D gel electrophoresis, reverse phase HPLC, capillary electrophoresis, isoelectric focusing and related hybrid techniques. Commonly used protein identification techniques include N-terminal Edman and mass spectrometry (electrospray [ESI] or matrix-assisted laser desorption ionization [MALDI] MS) and sophisticated database search programs, such as SEQUEST, to identify proteins in World Wide Web protein and nucleic acid databases from the MS-MS spectra of their peptides. Using a computer, the output of the mass spectrometry can be analyzed so as to link a gene and the particular protein for which it codes. This overall process is sometimes referred to as “functional genomics”.

For general information on proteome research, see, for example, J. S. Fruton, 1999, Proteins, Enzymes, Genes: The Interplay of Chemistry and Biology, Yale Univ. Pr.; Wilkins et al., 1997, Proteome Research: New Frontiers in Functional Genomics (Principles and Practice), Springer Verlag; A. J. Link, 1999, 2-D Proteome Analysis Protocols (Methods in Molecular Biology, 112, Humana Pr.); and Kamp et al., 1999, Proteome and Protein Analysis, Springer Verlag. Signal Transduction

See also, James, Peter, “Protein identification in the post-genome era: the rapid rise of proteomics”, Q. Rev. Biophysics, Vol. 30, No. 4, pp. 279-331 (1997).

Screening Peptides: Peptide Display Methods

The present invention is further directed to a method for generating a selected mutant polynucleotide sequence (or a population of selected polynucleotide sequences) typically in the form of amplified and/or cloned polynucleotides, whereby the selected polynucleotide sequences(s) possess at least one desired phenotypic characteristic (e.g., encodes a polypeptide, promotes transcription of linked polynucleotides, binds a protein, and the like) which can be selected for. One method for identifying hybrid polypeptides that possess a desired structure or functional property, such as binding to a predetermined biological macromolecule (e.g., a receptor), involves the screening of a large library of polypeptides for individual library members which possess the desired structure or functional property conferred by the amino acid sequence of the polypeptide.

One method of screening peptides involves the display of a peptide sequence, antibody, or other protein on the surface of a bacteriophage particle or cell. Generally, in these methods each bacteriophage particle or cell serves as an individual library member displaying a single species of displayed peptide in addition to the natural bacteriophage or cell protein sequences. Each bacteriophage or cell contains the nucleotide sequence information encoding the particular displayed peptide sequence; thus, the displayed peptide sequence can be ascertained by nucleotide sequence determination of an isolated library member.

A well-known peptide display method involves the presentation of a peptide sequence on the surface of a filamentous bacteriophage, typically as a fusion with a bacteriophage coat protein. The bacteriophage library can be incubated with an immobilized, predetermined macromolecule or small molecule (e.g., a receptor) so that bacteriophage particles which present a peptide sequence that binds to the immobilized macromolecule can be differentially partitioned from those that do not present peptide sequences that bind to the predetermined macromolecule. The bacteriophage particles (i.e., library members) which are bound to the immobilized macromolecule are then recovered and replicated to amplify the selected bacteriophage sub-population for a subsequent round of affinity enrichment and phage replication. After several rounds of affinity enrichment and phage replication, the bacteriophage library members that are thus selected are isolated and the nucleotide sequence encoding the displayed peptide sequence is determined, thereby identifying the sequence(s) of peptides that bind to the predetermined macromolecule (e.g., receptor). Such methods are further described in PCT patent publications WO 91/17271, WO 91/18980, WO 91/19818 and WO 93/08278.

The latter PCT publication describes a recombinant DNA method for the display of peptide ligands that involves the production of a library of fusion proteins with each fusion protein composed of a first polypeptide portion, typically comprising a variable sequence, that is available for potential binding to a predetermined macromolecule, and a second polypeptide portion that binds to DNA, such as the DNA vector encoding the individual fusion protein. When transformed host cells are cultured under conditions that allow for expression of the fusion protein, the fusion protein binds to the DNA vector encoding it. Upon lysis of the host cell, the fusion protein/vector DNA complexes can be screened against a predetermined macromolecule in much the same way as bacteriophage particles are screened in the phage-based display system, with the replication and sequencing of the DNA vectors in the selected fusion protein/vector DNA complexes serving as the basis for identification of the selected library peptide sequence(s).

The displayed peptide sequences can be of varying lengths, typically from 3-5000 amino acids long or longer, frequently from 5-100 amino acids long, and often from about 8-15 amino acids long. A library can comprise library members having varying lengths of displayed peptide sequence, or may comprise library members having a fixed length of displayed peptide sequence. Portions or all of the displayed peptide sequence(s) can be random, pseudorandom, defined set kernal, fixed, or the like. The present display methods include methods for in vitro and in vivo display of single-chain antibodies, such as nascent scFv on polysomes or scfv displayed on phage, which enable large-scale screening of scfv libraries having broad diversity of variable region sequences and binding specificities.

The present invention also provides random, pseudorandom, and defined sequence framework peptide libraries and methods for generating and screening those libraries to identify useful compounds (e.g., peptides, including single-chain antibodies) that bind to receptor molecules or epitopes of interest or gene products that modify peptides or RNA in a desired fashion. The random, pseudorandom, and defined sequence framework peptides are produced from libraries of peptide library members that comprise displayed peptides or displayed single-chain antibodies attached to a polynucleotide template from which the displayed peptide was synthesized. The mode of attachment may vary according to the specific aspect of the invention selected, and can include encapsulation in a phage particle or incorporation in a cell.

Screening That Utilizes in Vitro Translation Systems

An aspect of this invention provides for the use of in vitro translation during the step of screening. In vitro translation has been used to synthesize proteins of interest and has been proposed as a method for generating large libraries of peptides. These methods, generally comprising stabilized polysome complexes, are described further in PCT patent publications WO 88/08453, WO 90/05785, WO 90/07003, WO 91/02076, WO 91/05058, and WO 92/02536. Applicants have described methods in which library members comprise a fusion protein having a first polypeptide portion with DNA binding activity and a second polypeptide portion having the library member unique peptide sequence; such methods are suitable for use in cell-free in vitro selection formats, among others.

Affinity Enrichment

One aspect of this invention provides for the use of affinity enrichment which allows a very large library of peptides and single-chain antibodies to be screened and the polynucleotide sequence encoding the desired peptide(s) or single-chain antibodies to be selected. The polynucleotide can then be isolated and shuffled to recombine combinatorially the amino acid sequence of the selected peptide(s) (or predetermined portions thereof) or single-chain antibodies (or just VHI, VLI or CDR portions thereof). Using these methods, one can identify a peptide or single-chain antibody as having a desired binding affinity for a molecule and can exploit the process of shuffling to converge rapidly to a desired high-affinity peptide or scfv. The peptide or antibody can then be synthesized in bulk by conventional means for any suitable use (e.g., as a therapeutic or diagnostic agent).

A significant advantage of the present invention is that no prior information regarding an expected ligand structure is required to isolate peptide ligands or antibodies of interest. The peptide identified can have biological activity, which is meant to include at least specific binding affinity for a selected receptor molecule and, in some instances, will further include the ability to block the binding of other compounds, to stimulate or inhibit metabolic pathways, to act as a signal or messenger, to stimulate or inhibit cellular activity, and the like.

The present invention also provides a method for shuffling a pool of polynucleotide sequences selected by affinity screening a library of polysomes displaying nascent peptides (including single-chain antibodies) for library members which bind to a predetermined receptor (e.g., a mammalian proteinaceous receptor such as, for example, a peptidergic hormone receptor, a cell surface receptor, an intracellular protein which binds to other protein(s) to form intracellular protein complexes such as hetero-dimers and the like) or epitope (e.g., an immobilized protein, glycoprotein, oligosaccharide, and the like).

The invention also provides peptide libraries comprising a plurality of individual library members of the invention, wherein (1) each individual library member of said plurality comprises a sequence produced by shuffling of a pool of selected sequences, and (2) each individual library member comprises a variable peptide segment sequence or single-chain antibody segment sequence which is distinct from the variable peptide segment sequences or single-chain antibody sequences of other individual library members in said plurality (although some library members may be present in more than one copy per library due to uneven amplification, stochastic probability, or the like).

Antibody Display

The present method can be used to shuffle, by in vitro and/or in vivo recombination by any of the disclosed methods, and in any combination, polynucleotide sequences selected by antibody display methods, wherein an associated polynucleotide encodes a displayed antibody which is screened for a phenotype (e.g., for affinity for binding a predetermined antigen (ligand).

Various prokaryotic expression systems have been developed that can be manipulated to produce combinatorial antibody libraries which may be screened for high-affinity antibodies to specific antigens. Recent advances in the expression of antibodies in Escherichia coli and bacteriophage systems (see “alternative peptide display methods”, infra) have raised the possibility that virtually any specificity can be obtained by either cloning antibody genes from characterized hybridomas or by de novo selection using antibody gene libraries (e.g., from Ig cDNA).

Combinatorial libraries of antibodies have been generated in bacteriophage lambda expression systems which may be screened as bacteriophage plaques or as colonies of lysogens (Huse et al, 1989); Caton and Koprowski, 1990; Mullinax et al, 1990; Persson et al, 1991). Various aspects of bacteriophage antibody display libraries and lambda phage expression libraries have been described (Kang et al, 1991; Clackson et al, 1991; McCafferty et al, 1990; Burton et al, 1991; Hoogenboom et al, 1991; Chang et al, 1991; Breitling et al, 1991; Marks et al, 1991, p. 581; Barbas et al, 1992; Hawkins and Winter, 1992; Marks et al, 1992, p. 779; Marks et al, 1992, p. 16007; and Lowman et al, 1991; Lerner et al, 1992; all incorporated herein by reference). Typically, a bacteriophage antibody display library is screened with a receptor (e.g., polypeptide, carbohydrate, glycoprotein, nucleic acid) that is immobilized (e.g., by covalent linkage to a chromatography resin to enrich for reactive phage by affinity chromatography) and/or labeled (e.g., to screen plaque or colony lifts).

One aspect of the invention uses the so-called single-chain fragment variable (scfv) libraries (Marks et al, 1992, p. 779; Winter and Milstein, 1991; Clackson et al, 1991; Marks et al, 1991, p. 581; Chaudhary et al, 1990; Chiswell et al, 1992; McCafferty et al, 1990; and Huston et al, 1988). Various aspects of scfv libraries displayed on bacteriophage coat proteins have been described. Bacteriophage display of sclv have already yielded a variety of useful antibodies and antibody fusion proteins. A bispecific single chain antibody has been shown to mediate efficient tumor cell lysis (Gruber et al, 1994). Intracellular expression of an anti-Rev sclv has been shown to inhibit HIV-I virus replication in vitro (Duan et al, 1994), and intracellular expression of an anti-p21rar, scfv has been shown to inhibit meiotic maturation of Xenopus oocytes (Biocca et al, 1993). Recombinant scfv which can be used to diagnose HIV infection have also been reported, demonstrating the diagnostic utility of scfv (Lilley et al, 1994). Fusion proteins wherein an scFv is linked to a second polypeptide, such as a toxin or fibrinolytic activator protein, have also been reported (Holvost et al, 1992; Nicholls et al, 1993).

Various methods have been reported for increasing the combinatorial diversity of a scfv library to broaden the repertoire of binding species (idiotype spectrum). Enzymatic inverse PCR mutagenesis has been shown to be a simple and reliable method for constructing relatively large libraries of scfv site-directed hybrids (Stemmer et al, 1993), as has error-prone PCR and chemical mutagenesis (Deng et al, 1994). Riechmann (Riechmann et al, 1993) showed semi-rational design of an antibody scfv fragment using site-directed randomization by degenerate oligonucleotide PCR and subsequent phage display of the resultant scfv hybrids. Barbas (Barbas et al, 1992) attempted to circumvent the problem of limited repertoire sizes resulting from using biased variable region sequences by randomizing the sequence in a synthetic CDR region of a human tetanus toxoid-binding Fab.

Displayed peptide/polynucleotide complexes (library members) which encode a variable segment peptide sequence of interest or a single-chain antibody of interest are selected from the library by an affinity enrichment technique. This is accomplished by means of a immobilized macromolecule or epitope specific for the peptide sequence of interest, such as a receptor, other macromolecule, or other epitope species. Repeating the affinity selection procedure provides an enrichment of library members encoding the desired sequences, which may then be isolated for pooling and shuffling, for sequencing, and/or for further propagation and affinity enrichment.

The library members without the desired specificity are removed by washing. The degree and stringency of washing required will be determined for each peptide sequence or single-chain antibody of interest and the immobilized predetermined macromolecule or epitope. A certain degree of control can be exerted over the binding characteristics of the nascent peptide/DNA complexes recovered by adjusting the conditions of the binding incubation and the subsequent washing. The temperature, pH, ionic strength, divalent cations concentration, and the volume and duration of the washing will select for nascent peptide/DNA complexes within particular ranges of affinity for the immobilized macromolecule. Selection based on slow dissociation rate, which is usually predictive of high affinity, is often the most practical route. This may be done either by continued incubation in the presence of a saturating amount of free predetermined macromolecule, or by increasing the volume, number, and length of the washes. In each case, the rebinding of dissociated nascent peptide/DNA or peptide/RNA complex is prevented, and with increasing time, nascent peptide/DNA or peptide/RNA complexes of higher and higher affinity are recovered.

Additional modifications of the binding and washing procedures may be applied to find peptides with special characteristics. The affinities of some peptides are dependent on ionic strength or cation concentration. This is a useful characteristic for peptides that will be used in affinity purification of various proteins when gentle conditions for removing the protein from the peptides are required.

One variation involves the use of multiple binding targets (multiple epitope species, multiple receptor species), such that a scf library can be simultaneously screened for a multiplicity of scfv which have different binding specificities. Given that the size of a scfv library often limits the diversity of potential scfv sequences, it is typically desirable to us scfv libraries of as large a size as possible. The time and economic considerations of generating a number of very large polysome scFv-display libraries can become prohibitive. To avoid this substantial problem, multiple predetermined epitope species (receptor species) can be concomitantly screened in a single library, or sequential screening against a number of epitope species can be used. In one variation, multiple target epitope species, each encoded on a separate bead (or subset of beads), can be mixed and incubated with a polysome-display scfv library under suitable binding conditions. The collection of beads, comprising multiple epitope species, can then be used to isolate, by affinity selection, scfv library members. Generally, subsequent affinity screening rounds can include the same mixture of beads, subsets thereof, or beads containing only one or two individual epitope species. This approach affords efficient screening, and is compatible with laboratory automation, batch processing, and high throughput screening methods.

Expression Systems

The DNA expression constructs will typically include an expression control DNA sequence operably linked to the coding sequences, including naturally-associated or heterologous promoter regions. The expression control sequences can be eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the mutant’ “engineered” antibodies.

The DNA sequences will be expressed in hosts after the sequences have been operably linked to an expression control sequence (i.e., positioned to ensure the transcription and translation of the structural gene). These expression vectors are typically replicable in the host organisms either as episomes or as an integral part of the host chromosomal DNA. Commonly, expression vectors will contain selection markers, e.g., tetracycline or neomycin, to permit detection of those cells transformed with the desired DNA sequences (see, e.g., U.S. Pat. No. 4,704,362).

In addition to eukaryotic microorganisms such as yeast, mammalian tissue cell culture may also be used to produce the polypeptides of the present invention (see Winnacker, 1987), which is incorporated herein by reference). Eukaryotic cells can be used because a number of suitable host cell lines capable of secreting intact immunoglobulins have been developed in the art, and include the CHO cell lines, various COS cell lines, HeLa cells, and myeloma cell lines, or transformed B cells or hybridomas. Expression vectors for these cells can include expression control sequences, such as an origin of replication, a promoter, an enhancer (Queen et al, 1986), and necessary processing information sites, such as ribosome binding sites, RNA splice sites, polyadenylation sites, and transcriptional terminator sequences. Expression control sequences can be promoters derived from immunoglobulin genes, cytomegalovirus, SV40, Adenovirus, Bovine Papilloma Virus, and the like.

Eukaryotic DNA transcription can be increased by inserting an enhancer sequence into the vector. Enhancers are cis-acting sequences of between 10 to 300 bp that increase transcription by a promoter. Enhancers can effectively increase transcription when either 5′ or 3′ to the transcription unit. They are also effective if located within an intron or within the coding sequence itself. Typically, viral enhancers are used, including SV40 enhancers, cytomegalovirus enhancers, polyoma enhancers, and adenovirus enhancers. Enhancer sequences from mammalian systems are also commonly used, such as the mouse immunoglobulin heavy chain enhancer.

Mammalian expression vector systems will also typically include a selectable marker gene. Examples of suitable markers include, the dihydrofolate reductase gene (DHFR), the thymidine kinase gene (TK), or prokaryotic genes conferring drug resistance. The first two marker genes can use mutant cell lines that lack the ability to grow without the addition of thymidine to the growth medium. Transformed cells can then be identified by their ability to grow on non-supplemented media. Examples of prokaryotic drug resistance genes useful as markers include genes conferring resistance to G418, mycophenolic acid and hygromycin.

The vectors containing the DNA segments of interest can be transferred into the host cell by well-known methods, depending on the type of cellular host. For example, calcium chloride transfection is commonly utilized for prokaryotic cells, whereas calcium phosphate treatment. lipofection, or electroporation may be used for other cellular hosts. Other methods used to transform mammalian cells include the use of Polybrene, protoplast fusion, liposomes, electroporation, and micro-injection (see, generally, Sambrook et al, 1982 and 1989).

Once expressed, the antibodies, individual mutated immunoglobulin chains, mutated antibody fragments, and other immunoglobulin polypeptides of the invention can be purified according to standard procedures of the art, including ammonium sulfate precipitation, fraction column chromatography, gel electrophoresis and the like; see, e.g., Scopes, 1982. Once purified, partially or to homogeneity as desired, the polypeptides may then be used therapeutically or in developing and performing assay procedures, immunofluorescent stainings, and the like (see, generally, Lefkovits and Pemis, 1979 and 1981; Lefkovits, 1997).

Two-Hybrid Based Screening Assays

This invention provides a two-hybrid screening system to identify library members which bind a predetermined polypeptide sequence. The selected library members are pooled and shuffled by in vitro and/or in vivo recombination. The shuffled pool can then be screened in a yeast two hybrid system to select library members which bind said predetermined polypeptide sequence (e. g., and SH2 domain) or which bind an alternate predetermined polypeptide sequence (e.g., an SH2 domain from another protein species).

An approach to identifying polypeptide sequences which bind to a predetermined polypeptide sequence has been to use a so-called “two-hybrid” system wherein the predetermined polypeptide sequence is present in a fusion protein (Chien et al, 1991). This approach identifies protein-protein interactions in vivo through reconstitution of a transcriptional activator (Fields and Song, 1989), the yeast Gal4 transcription protein. Typically, the method is based on the properties of the yeast Gal4 protein, which consists of separable domains responsible for DNA-binding and transcriptional activation. Polynucleotides encoding two hybrid proteins, one consisting of the yeast Gal4 DNA-binding domain fused to a polypeptide sequence of a known protein and the other consisting of the Gal4 activation domain fused to a polypeptide sequence of a second protein, are constructed and introduced into a yeast host cell. Intermolecular binding between the two fusion proteins reconstitutes the Gal4 DNA-binding domain with the Gal4 activation domain, which leads to the transcriptional activation of a reporter gene (e.g., lacz, HIS3) which is operably linked to a Gal4 binding site. Typically, the two-hybrid method is used to identify novel polypeptide sequences which interact with a known protein (Silver and Hunt, 1993; Durfee et al, 1993; Yang et al, 1992; Luban et al, 1993; Hardy et al, 1992; Bartel et al, 1993; and Vojtek et al, 1993). However, variations of the two-hybrid method have been used to identify mutations of a known protein that affect its binding to a second known protein (Li and Fields, 1993; Lalo et al, 1993; Jackson et al, 1993; and Madura et al, 1993). Two-hybrid systems have also been used to identify interacting structural domains of two known proteins (Bardwell et al, 1993; Chakrabarty et al, 1992; Staudinger et al, 1993; and Milne and Weaver 1993) or domains responsible for oligomerization of a single protein (Iwabuchi et al, 1993; Bogerd et al, 1993). Variations of two-hybrid systems have been used to study the in vivo activity of a proteolytic enzyme (Dasmahapatra et al, 1992). Alternatively, an E. coli/BCCP interactive screening system (Germino et al, 1993; Guarente, 1993) can be used to identify interacting protein sequences (i.e., protein sequences which heterodimerize or form higher order heteromultimers). Sequences selected by a two-hybrid system can be pooled and shuffled and introduced into a two-hybrid system for one or more subsequent rounds of screening to identify polypeptide sequences which bind to the hybrid containing the predetermined binding sequence. The sequences thus identified can be compared to identify consensus sequence(s) and consensus sequence kernals.

Improved Methods for Cellular Engineering, Protein Expression Profiling, Differential Labeling of Peptides, and Novel Reagents Therefore

The invention relates to peptide chemistry, proteomics, and mass spectrometry technology. In particular, the invention provides novel methods for determining polypeptide profiles and protein expression variations, as with proteome analyses. The present invention provides methods of simultaneously identifying and quantifying individual proteins in complex protein mixtures by selective differential labeling of amino acid residues followed by chromatographic and mass spectrographic analysis.

The diagnosis and treatment, as well as the predisposition of, a variety of diseases and disorders may often be accomplished through identification and quantitative measurement of polypeptide expression variations between different cell types and cell states. Biochemical pathways and metabolic networks can also be analyzed by globally and quantitatively measuring protein expression in various cell types and biological states (see, e.g., Ideker (2001) Science 292:929-934).

State-of-the-art techniques such as liquid-chromatography-electrospray-ionization tandem mass spectrometry have, in conjunction with database-searching computer algorithms, revolutionized the analysis of biochemical species from complex biological mixtures. With these techniques, it is now possible to perform high-throughput protein identification at picomolar to subpicomolar levels from complex mixtures of biological molecules (see, e.g., Dongre (1997) Trends Biotechnol. 15:418-425).

One such method is based on a class of chemical reagents termed isotope-coded affinity tags (ICATs) and tandem mass spectrometry. The method labels multiple cysteinyl residues and uses stable isotope dilution techniques. For example, Gygi (1999) Nat. Biotechnol. 10:994-999, compared protein expression in a yeast using ethanol or galactose as a carbon source. The measured differences in protein expression correlated with known yeast metabolic function under glucose-repressed conditions.

In another technique, two different protein mixtures for quantitative comparison are digested to peptide mixtures, the peptides mixtures are separately methylated using either d0- or d3-methanol, the mixtures of methylated peptide combined and subjected to microcapillary HPLC-MS/MS (see, e.g., Goodlett, D. R., et al., (2000) “Differential stable isotope labeling of peptides for quantitation and de novo sequence derivation,” 49th ASMS; Zhou, H; Watts, J D; Aebersold, R. A systematic approach to the analysis of protein phosphorylation.; Comment In: Nat Biotechnol. April 2001; 19(4):317-8; Nature Biotechnology April 2001, 19(4):375-8). Parent proteins of methylated peptides are identified by correlative database searching of fragment ion spectra using a computer program assisted paradigms or automated de novo sequencing that compares all tandem mass spectra of d0- and d3-methylated peptide ion pairs. In Goodlett (2000) supra, ratios of proteins in two different mixtures were calculated for d0- to d3-methylated peptide pairs. However, there are several limitations to this approach, including: use of differential labeling reagents, which relied on stable isotopes, which are expensive, and not flexible to differential labeling of more than two mixtures of peptides; labeling methods limited only to methylation of carboxy-termini; protein expression profiling limited to duplex comparison; one dimensional capillary HPLC chromatography was employed to separate peptides, which doesn't has enough capacity and resolving power for complex mixtures of peptides.

In one aspect this invention provides a method for identifying proteins by differential labeling of peptides, the method comprising the following steps: (a) providing a sample comprising a polypeptide; (b) providing a plurality of labeling reagents which differ in molecular mass that can generate differential labeled peptides that do not differ in chromatographic retention properties and do not differ in ionization and detection properties in mass spectrographic analysis, wherein the differences in molecular mass are distinguishable by mass spectrographic analysis; (c) fragmenting the polypeptide into peptide fragments by enzymatic digestion or by non-enzymatic fragmentation; (d) contacting the labeling reagents of step (b) with the peptide fragments of step (c), thereby labeling the peptides with the differential labeling reagents; (e) separating the peptides by chromatography to generate an eluate; (f) feeding the eluate of step (e) into a mass spectrometer and quantifying the amount of each peptide and generating the sequence of each peptide by use of the mass spectrometer; (g) inputting the sequence to a computer program product which compares the inputted sequence to a database of polypeptide sequences to identify the polypeptide from which the sequenced peptide originated.

In one aspect, the sample of step (a) comprises a cell or a cell extract. The method can further comprise providing two or more samples comprising a polypeptide. One or more of the samples can be derived from a wild type cell and one sample can be derived from an abnormal or a modified cell. The abnormal cell can be a cancer cell. The modified cell can be a cell that is mutagenized &/or treated with a chemical, a physiological factor, or the presence of another organism (including, e.g. a eukaryotic organism, prokaryotic organism, virus, vector, prion, or part thereof), &/or exposed to an environmental factor or change or physical force (including, e.g., sound, light, heat, sonication, and radiation). The modification can be genetic change (including, for example, a change in DNA or RNA sequence or content) or otherwise.

In one aspect, the method further comprises purifying or fractionating the polypeptide before the fragmenting of step (c). The method can further comprise purifying or fractionating the polypeptide before the labeling of step (d). The method can further comprise purifying or fractionating the labeled peptide before the chromatography of step (e). In alternative aspects, the purifying or fractionating comprises a method selected from the group consisting of size exclusion chromatography, size exclusion chromatography, HPLC, reverse phase HPLC and affinity purification. In one aspect, the method further comprises contacting the polypeptide with a labeling reagent of step (b) before the fragmenting of step (c).

In one aspect, the labeling reagent of step (b) comprises the general formulae selected from the group consisting of: Z^{A}OH and Z^{B}OH, to esterify peptide C-terminals and/or Glu and Asp side chains; Z^{A}NH2 and Z^{B}NH₂, to form amide bond with peptide C-terminals and/or Glu and Asp side chains; and Z^{A}CO₂H and Z^{B}CO₂H. to form amide bond with peptide N-terminals and/or Lys and Arg side chains; wherein Z^{A} and Z^{B} independently of one another comprise the general formula R-Z¹-A¹-Z² -A²-Z³-A³-Z⁴-A⁴-, Z¹, Z², Z³, and Z⁴ independently of one another, are selected from the group consisting of nothing, O, OC(O), OC(S), OC(O)O, OC(O)NR, OC(S)NR, OSiRR¹, S, SC(O), SC(S), SS, S(O), S(O₂), NR, NRR¹⁺, C(O), C(O)O, C(S), C(S)O, C(O)S, C(O)NR, C(S)NR, SiRR¹, (Si(RR¹)O)ₙ, SnRR¹, Sn(RR¹)O, BR(OR¹), BRR¹, B(OR)(OR¹), OBR(OR¹), OBRR¹, and OB(OR)(OR¹), and R and R¹ is an alkyl group, A¹, A², A³, and A⁴ independently of one another, are selected from the group consisting of nothing or (CRR¹)ₙ, wherein R, R¹, independently from other R and R¹ in Z¹ to Z⁴ and independently from other R and R¹ in A¹ to A⁴, are selected from the group consisting of a hydrogen atom, a halogen atom and an alkyl group; “n” in Z¹ to Z⁴, independent of n in A¹ to A⁴, is an integer having a value selected from the group consisting of 0 to about 51; 0 to about 41; 0 to about 31; 0 to about 21, 0 to about 11 and 0 to about 6.

In one aspect, the alkyl group (see definition below) is selected from the group consisting of an alkenyl, an alkynyl and an aryl group. One or more C—C bonds from (CRR¹)ₙ can be replaced with a double or a triple bond; thus, in alternative aspects, an R or an R¹ group is deleted. The (CRR¹)ₙ can be selected from the group consisting of an o-arylene, an m-arylene and a p-arylene, wherein each group has none or up to 6 substituents. The (CRR¹)ₙ can be selected from the group consisting of a carbocyclic, a bicyclic and a tricyclic fragment, wherein the fragment has up to 8 atoms in the cycle with or without a heteroatom selected from the group consisting of an O atom, a N atom and an S atom.

In one aspect, two or more labeling reagents have the same structure but a different isotope composition. For example, in one aspect, Z^{A} has the same structure as Z^{B}, while Z^{A} has a different isotope composition than Z^{B}. In alternative aspects, the isotope is boron-10 and boron-11; carbon-12 and carbon-13; nitrogen-14 and nitrogen-15; and, sulfur-32 and sulfur-34. In one aspect, where the isotope with the lower mass is x and the isotope with the higher mass is y, and x and y are integers, x is greater than y.

In alternative aspects, x and y are between 1 and about 11, between 1 and about 21, between 1 and about 31, between 1 and about 41, or between 1 and about 51.

In one aspect, the labeling reagent of step (b) comprises the general formulae selected from the group consisting of: CD₃(CD₂)ₙOH/CH₃(CH₂)ₙOH, to esterify peptide C-terminals, where n=0, 1, 2 or y; CD₃(CD₂)ₙNH₂/CH₃(CH₂)ₙNH₂, to form amide bond with peptide C-terminals, where n=0, 1, 2 or y; and, D(CD₂)ₙCO₂H/H(CH₂)ₙCO₂H, to form amide bond with peptide N-terminals, where n=0, 1, 2 or y; wherein D is a deuteron atom, and y is an integer selected from the group consisting of about 51; about 41; about 31; about 21, about 11; about 6 and between about 5 and 51.

In one aspect, the labeling reagent of step (b) can comprise the general formulae selected from the group consisting of: Z^{A}OH and Z^{B}OH to esterify peptide C-terminals; Z^{A}NH₂/Z^{B}NH2 to form an amide bond with peptide C-terminals; and, Z^{A}CO₂H /Z^{B}CO₂H to form an amide bond with peptide N-terminals; wherein Z^{A} and Z^{B} have the general formula R-Z¹-A¹-Z²-A²-Z³-A³-Z⁴-A⁴-; Z¹, Z², Z³, and Z⁴, independently of one another, are selected from the group consisting of nothing, O, OC(O), OC(S), OC(O)O, OC(O)NR, OC(S)NR, OSiRR¹, S, SC(O), SC(S), SS, S(O), S(O₂), NR, NRR¹⁺, C(O), C(O)O, C(S), C(S)O, C(O)S, C(O)NR, C(S)NR, SiRR¹, (Si(RR¹)O)n, SNRR¹, Sn(RR¹)O, BR(OR¹), BRR¹, B(OR)(OR¹), OBR(OR¹), OBRR¹, and OB(OR)(OR¹); A¹, A², A³, and A⁴, independently of one another, are selected from the group consisting of nothing and the general formulae (CRR¹)n, and, R and R¹ is an alkyl group.

In one aspect, a single C—C bond in a (CRR¹)n group is replaced with a double or a triple bond; thus, the R and R¹ can be absent. The (CRR¹)n can comprise a moiety selected from the group consisting of an o-arylene, an m-arylene and a p-arylene, wherein the group has none or up to 6 substituents. The group can comprise a carbocyclic, a bicyclic, or a tricyclic fragments with up to 8 atoms in the cycle, with or without a heteroatom selected from the group consisting of an O atom, an N atom and an S atom. In one aspect, R, R¹, independently from other R and R¹ in Z¹-Z⁴ and independently from other R and R¹ in A¹-A⁴, are selected from the group consisting of a hydrogen atom, a halogen and an alkyl group. The alkyl group (see definition below) can be an alkenyl, an alkynyl or an aryl group.

In one aspect, the “n” in Z¹-Z⁴ is independent of n in A¹-A⁴ and is an integer selected from the group consisting of about 51; about 41; about 31; about 21, about 11 and about 6. In one aspect, Z^{A} has the same structure a Z^{B} but Z^{A} further comprises x number of —CH₂— fragment(s) in one or more A¹-A⁴ fragments, wherein x is an integer. In one aspect, Z^{A} has the same structure a Z^{B} but Z^{A} further comprises x number of —CF₂— fragment(s) in one or more A¹-A⁴ fragments, wherein x is an integer. In one aspect, Z^{A} comprises x number of protons and Z^{B} comprises y number of halogens in the place of protons, wherein x and y are integers. In one aspect, Z^{A} contains x number of protons and Z^{B} contains y number of halogens, and there are x-y number of protons remaining in one or more A¹-A⁴ fragments, wherein x and y are integers. In one aspect, Z^{A} further comprises x number of —O— fragment(s) in one or more A¹-A⁴ fragments, wherein x is an integer. In one aspect, Z^{A} further comprises x number of —S— fragment(s) in one or more A¹-A⁴ fragments, wherein x is an integer. In one aspect, Z^{A} further comprises x number of —O— fragment(s) and Z^{B} further comprises y number of —S— fragment(s) in the place of —O— fragment(s), wherein x and y are integers. In one aspect, Z^{A} further comprises x-y number of —O— fragment(s) in one or more A¹-A⁴ fragments, wherein x and y are integers.

In alternative aspects, x and y are integers selected from the group consisting of between 1 about 51; between 1 about 41; between 1 about 31; between 1 about 21, between 1 about 11 and between 1 about 6, wherein x is greater than y.

In one aspect, the labeling reagent of step (b) comprises the general formulae selected from the group consisting of: CH₃(CH₂)ₙOH/CH₃(CH₂)ₙ₊ₘOH, to esterify peptide C-terminals, where n=0, 1, 2, . . . , y; m=1, 2, . . . , y; CH₃(CH₂)ₙ NH₂/CH₃(CH₂)ₙ₊ₘNU₂, to form amide bond with peptide C-terminals, where n=0, 1, 2, . . . , y; m=1, 2, . . . , y; and, H(CH₂)ₙCO₂H/H(CH₂)ₙ₊ₘCO₂H, to form amide bond with peptide N-terminals, where n=0, 1, 2, . . . , y; m=1, 2, . . . , y; wherein n, m and y are integers. In one aspect, n, m and y are integers selected from the group consisting of about 51; about 41; about 31; about 21, about 11; about 6 and between about 5 and 51.

In one aspect, the separating of step (e) comprises a liquid chromatography system, such as a multidimensional liquid chromatography or a capillary chromatography system. In one aspect, the mass spectrometer comprises a tandem mass spectrometry device. In one aspect, the method further comprises quantifying the amount of each polypeptide or each peptide.

The invention provides a method for defining the expressed proteins associated with a given cellular state, the method comprising the following steps: (a) providing a sample comprising a cell in the desired cellular state; (b) providing a plurality of labeling reagents which differ in molecular mass that can generate differential labeled peptides that do not differ in chromatographic retention properties and do not differ in ionization and detection properties in mass spectrographic analysis, wherein the differences in molecular mass are distinguishable by mass spectrographic analysis; (c) fragmenting polypeptides derived from the cell into peptide fragments by enzymatic digestion or by non-enzymatic fragmentation; (d) contacting the labeling reagents of step (b) with the peptide fragments of step (c), thereby labeling the peptides with the differential labeling reagents; (e) separating the peptides by chromatography to generate an eluate; (f) feeding the eluate of step (e) into a mass spectrometer and quantifying the amount of each peptide and generating the sequence of each peptide by use of the mass spectrometer; (g) inputting the sequence to a computer program product which compares the inputted sequence to a database of polypeptide sequences to identify the polypeptide from which the sequenced peptide originated, thereby defining the expressed proteins associated with the cellular state.

The invention provides a method for quantifying changes in protein expression between at least two cellular states, the method comprising the following steps: (a) providing at least two samples comprising cells in a desired cellular state; (b) providing a plurality of labeling reagents which differ in molecular mass that can generate differential labeled peptides that do not differ in chromatographic retention properties and do not differ in ionization and detection properties in mass spectrographic analysis, wherein the differences in molecular mass are distinguishable by mass spectrographic analysis; (c) fragmenting polypeptides derived from the cells into peptide fragments by enzymatic digestion or by non-enzymatic fragmentation; (d) contacting the labeling reagents of step (b) with the peptide fragments of step (c), thereby labeling the peptides with the differential labeling reagents, wherein the labels used in one same are different from the labels used in other samples; (e) separating the peptides by chromatography to generate an eluate; (f) feeding the eluate of step (e) into a mass spectrometer and quantifying the amount of each peptide and generating the sequence of each peptide by use of the mass spectrometer; (g) inputting the sequence to a computer program product which identifies from which sample each peptide was derived, compares the inputted sequence to a database of polypeptide sequences to identify the polypeptide from which the sequenced peptide originated, and compares the amount of each polypeptide in each sample, thereby quantifying changes in protein expression between at least two cellular states.

The invention provides a method for identifying proteins by differential labeling of peptides, the method comprising the following steps: (a) providing a sample comprising a polypeptide; (b) providing a plurality of labeling reagents which differ in molecular mass but do not differ in chromatographic retention properties and do not differ in ionization and detection properties in mass spectrographic analysis, wherein the differences in molecular mass are distinguishable by mass spectrographic analysis; (c) fragmenting the polypeptide into peptide fragments by enzymatic digestion or by non-enzymatic fragmentation; (d) contacting the labeling reagents of step (b) with the peptide fragments of step (c), thereby labeling the peptides with the differential labeling reagents; (e) separating the peptides by multidimensional liquid chromatography to generate an eluate; (f) feeding the eluate of step (e) into a tandem mass spectrometer and quantifying the amount of each peptide and generating the sequence of each peptide by use of the mass spectrometer; (g) inputting the sequence to a computer program product which compares the inputted sequence to a database of polypeptide sequences to identify the polypeptide from which the sequenced peptide originated.

The invention provides a chimeric labeling reagent comprising (a) a first domain comprising a biotin; and (b) a second domain comprising a reactive group capable of covalently binding to an amino acid, wherein the chimeric labeling reagent comprises at least one isotope. The isotope(s) can be in the first domain or the second domain. For example, the isotope(s) can be in the biotin.

In alternative aspects, the isotope can be a deuterium isotope, a boron-10 or boron-11 isotope, a carbon-12 or a carbon-13 isotope, a nitrogen-14 or a nitrogen-15 isotope, or, a sulfur-32 or a sulfur-34 isotope. The chimeric labeling reagent can comprise two or more isotopes. The chimeric labeling reagent reactive group capable of covalently binding to an amino acid can be a succimide group, an isothiocyanate group or an isocyanate group. The reactive group can be capable of covalently binding to an amino acid binds to a lysine or a cysteine.

The chimeric labeling reagent can further comprising a linker moiety linking the biotin group and the reactive group. The linker moiety can comprise at least one isotope. In one aspect, the linker is a cleavable moiety that can be cleaved by, e.g., enzymatic digest or by reduction.

The invention provides a method of comparing relative protein concentrations in a sample comprising (a) providing a plurality of differential small molecule tags, wherein the small molecule tags are structurally identical but differ in their isotope composition, and the small molecules comprise reactive groups that covalently bind to cysteine or lysine residues or both; (b) providing at least two samples comprising polypeptides; (c) attaching covalently the differential small molecule tags to amino acids of the polypeptides; (d) determining the protein concentrations of each sample in a tandem mass spectrometer; and, (d) comparing relative protein concentrations of each sample. In one aspect, the sample comprises a complete or a fractionated cellular sample.

In one aspect of the method, the differential small molecule tags comprise a chimeric labeling reagent comprising (a) a first domain comprising a biotin; and, (b) a second domain comprising a reactive group capable of covalently binding to an amino acid, wherein the chimeric labeling reagent comprises at least one isotope. The isotope can be a deuterium isotope, a boron-10 or boron-11 isotope, a carbon-12 or a carbon-13 isotope, a nitrogen-14 or a nitrogen-15 isotope, or, a sulfur-32 or a sulfur-34 isotope. The chimeric labeling reagent can comprise two or more isotopes. The reactive group can be capable of covalently binding to an amino acid is selected from the group consisting of a succimide group, an isothiocyanate group and an isocyanate group.

The invention provides a method of comparing relative protein concentrations in a sample comprising (a) providing a plurality of differential small molecule tags, wherein the differential small molecule tags comprise a chimeric labeling reagent comprising (i) a first domain comprising a biotin; and, (ii) a second domain comprising a reactive group capable of covalently binding to an amino acid, wherein the chimeric labeling reagent comprises at least one isotope; (b) providing at least two samples comprising polypeptides; (c) attaching covalently the differential small molecule tags to amino acids of the polypeptides; (d) isolating the tagged polypeptides on a biotin-binding column by binding tagged polypeptides to the column, washing non-bound materials off the column, and eluting tagged polypeptides off the column; (e) determining the protein concentrations of each sample in a tandem mass spectrometer; and, (f) comparing relative protein concentrations of each sample.

The invention provides methods for simultaneously identifying individual proteins in complex mixtures of biological molecules and quantifying the expression levels of those proteins, e.g., proteome analyses. The methods compare two or more samples of proteins, one of which can be considered as the standard sample and all others can be considered as samples under investigation. The proteins in the standard and investigated samples are subjected separately to a series of chemical modifications, i.e., differential chemical labeling, and fragmentation, e.g., by proteolytic digestion and/or other enzymatic reactions or physical fragmenting methodologies. The chemical modifications can be done before, or after, or before and after fragmentation/digestion of the polypeptide into peptides.

Peptides derived from the standard and the investigated samples are labeled with chemical residues of different mass, but of similar properties, such that peptides with the same sequence from both samples are eluted together in the separation procedure and their ionization and detection properties regarding the mass spectrometry are very similar. Differential chemical labeling can be performed on reactive functional groups on some or all of the carboxy- and/or amino-termini of proteins and peptides and/or on selected amino acid side chains. A combination of chemical labeling, proteolytic digestion and other enzymatic reaction steps, physical fragmentation and/or fractionation can provide access to a variety of residues to general different specifically labeled peptides to enhance the overall selectivity of the procedure.

The standard and the investigated samples are combined, subjected to multidimensional chromatographic separation, and analyzed by mass spectrometry methods. Mass spectrometry data is processed by special software, which allows for identification and quantification of peptides and proteins.

Depending on the complexity and composition of the protein samples, it may be desirable, or be necessary, to perform protein fractionation using such methods as size exclusion, ion exchange, reverse phase, or other methods of affinity purifications prior to one or more chemical modification steps, proteolytic digestion or other enzymatic reaction steps, or physical fragmentation steps.

The combined mixtures of peptides are first separated by a chromatography method, such as a multidimensional liquid chromatography, system, before being fed into a coupled mass spectrometry device, such as a tandem mass spectrometry device. The combination of multidimensional liquid chromatography and tandem mass spectrometry can be called “LC-LC-MS/MS.” LC-LC-MS/MS was first developed by Link A. and Yates J. R., as described, e.g., by Link (1999) Nature Biotechnology 17:676-682; Link (1999) Electrophoresis 18:1314-1334; Washburn, M P; Wolters, D; Yates, J R, Nature Biotechnology March 2001, 19(3):242-7.

In practicing the methods of the invention, proteins can be first substantially or partially isolated from the biological samples of interest. The polypeptides can be treated before selective differential labeling; for example, they can be denatured, reduced, preparations can be desalted, and the like. Conversion of samples of proteins into mixtures of differentially labeled peptides can include preliminary chemical and/or enzymatic modification of side groups and/or termini; proteolytic digestion or fragmentation; post-digestion or post-fragmentation chemical and/or enzymatic modification of side groups and/or termini.

The differentially modified polypeptides and peptides are then combined into one or more peptide mixtures. Solvent or other reagents can be removed, neutralized or diluted, if desired or necessary. The buffer can be modified, or, the peptides can be re-dissolved in one or more different buffers, such as a “MudPIT” (see below) loading buffer. The peptide mixture is then loaded onto chromatography column, such as a liquid chromatography column, a 2D capillary column or a multidimensional chromatography column, to generate an eluate.

The eluate is fed into a mass spectrometer, such as a tandem mass spectrometer. In one aspect, an LC ESI MS and MS/MS analysis is complete. Finally, data output is processed by appropriate software using database searching and data analysis.

In practicing the methods of the invention, high yields of peptides can generated for mass spectrograph analysis. Two or more samples can be differentially labeled by selective labeling of each sample. Peptide modifications, i.e., labeling, are stable. Reagents having differing masses or reactive groups can be chosen to maximize the number of reactive groups and differentially labeled samples, thus allowing for a multiplex analysis of sample, polypeptides and peptides. In one aspect, a “MudPIT” protocol is used for peptide analysis, as described herein. The methods of the invention can be fully automated and can essentially analyze every protein in a sample.

Definitions

Unless defined otherwise, all technical and scientific terms used herein have the meaning commonly understood by a person skilled in the art to which this invention belongs. As used herein, the following terms have the meanings ascribed to them unless specified otherwise.

As used herein, the term “alkyl” is used to refer to a genus of compounds including branched or unbranched, saturated or unsaturated, monovalent hydrocarbon radicals, including substituted derivatives and equivalents thereof. In one aspect, the hydrocarbons have from about 1 to about 100 carbons, about 1 to about 50 carbons or about 1 to about 30 carbons, about 1 to about 20 carbons, about 1 to about 10 carbons. When the alkyl group has from about 1 to 6 carbon atoms, it is referred to as a “lower alkyl.” Suitable alkyl radicals include, e.g., structures containing one or more methylene, methine and/or methyne groups arranged in acyclic and/or cyclic forms. Branched structures have a branching motif similar to isopropyl, tert-butyl isobutyl, 2-ethylpropyl, etc. As used herein, the term encompasses “substituted alkyls.” “Substituted alkyl” refers to alkyl as just described including one or more functional groups such as lower alkyl, aryl, acyl, halogen (i.e., alkylhalos, e.g., CF3), hydroxy, amino, alkoxy, alkylamino, acylamino, thioamido, acyloxy, aryloxy, arylamino, aryloxyalkyl, mercapto, thia, aza, oxo, both saturated and unsaturated cyclic hydrocarbons, heterocycles and the like. These groups may be attached to any carbon of the alkyl moiety. Additionally, these groups may be pendent from, or integral to, the alkyl chain.

The term “alkoxy” is used herein to refer to the to a COR group, where R is a lower alkyl, substituted lower alkyl, aryl, substituted aryl, arylalkyl or substituted arylalkyl wherein the alkyl, aryl, substituted aryl, arylalkyl and substituted arylalkyl groups are as described herein. Suitable alkoxy radicals include, for example, methoxy, ethoxy, phenoxy, substituted phenoxy, benzyloxy phenethyloxy, tert.-butoxy, etc. The term “aryl” is used herein to refer to an aromatic substituent that may be a single aromatic ring or multiple aromatic rings which are fused together, linked covalently, or linked to a common group such as a methylene or ethylene moiety. The common linking group may also be a carbonyl as in benzophenone. The aromatic ring(s) may include phenyl, naphthyl, biphenyl, diphenylmethyl and benzophenone among others. The term “aryl” encompasses “arylalkyl.” “Substituted aryl” refers to aryl as just described including one or more functional groups such as lower alkyl, acyl, halogen, alkylhalos (e.g., CF3), hydroxy, amino, alkoxy, alkylamino, acylamino, acyloxy, phenoxy, mercapto and both saturated and unsaturated cyclic hydrocarbons which are fused to the aromatic ring(s), linked covalently or linked to a common group such as a methylene or ethylene moiety. The linking group may also be a carbonyl such as in cyclohexyl phenyl ketone. The term “substituted aryl” encompasses “substituted arylalkyl.”

The term “arylalkyl” is used herein to refer to a subset of “aryl” in which the aryl group is further attached to an alkyl group, as defined herein.

The term “biotin” as used herein refers to any natural or synthetic biotin or variant thereof, which are well known in the art; ligands for biotin, and ways to modify the affinity of biotin for a ligand, are also well known in the art; see, e.g., U.S. Pat. Nos. 6,242,610; 6,150,123; 6,096,508; 6,083,712; 6,022,688; 5,998,155; 5,487,975.

The phrase “labeling reagents which . . . do not differ in ionization and detection properties in mass spectrographic analysis” means that the amount and/or mass sequence of the labeling reagents can be detected using the same mass spectrographic conditions and detection devices.

The term “polypeptide” includes natural and synthetic polypeptides, or mimetics, which can be either entirely composed of synthetic, non-natural analogues of amino acids, or, they can be chimeric molecules of partly natural peptide amino acids and partly non-natural analogs of amino acids. The term “polypeptide” as used herein includes proteins and peptides of all sizes.

The term “sample” as used herein includes any polypeptide-containing sample, including samples from natural sources, or, entirely synthetic samples.

The term “column” as used herein means any substrate surface, including beads, filaments, arrays, tubes and the like.

The phrase “do not differ in chromatographic retention properties” as used herein means that two compositions have substantially, but not necessary exactly, the same retention properties in a chromatograph, such as a liquid chromatograph. For example, two compositions do not differ in chromatographic retention properties if they elute together, i.e., they elute in what a skilled artisan would consider the same elution fraction.

Differential Labeling of Peptides and Polypeptides

In practicing the methods of the invention, proteins and peptides are subjected to a series of chemical modifications, i.e., differential chemical labeling. The chemical modifications can be done before, or after, or before and after fragmentation/digestion of the polypeptide into peptides. Differential labeling reagents can differ in their isotope composition (i.e., isotopical reagents), in their structural composition (i.e., homologous reagents), but by a rather small fragment which change does not alter the properties stated above, i.e., the labeling reagent differ in molecular mass but do not differ in chromatographic retention properties and do not differ in ionization and detection properties in mass spectrographic analysis, and the differences in molecular mass are distinguishable by mass spectrographic analysis.

In one aspect of the invention, mixtures of polypeptides and/or peptides coming from the “standard” protein sample and the “investigated” protein sample(s) are labeled separately with differential reagents, or, one sample is labeled and other sample remains unlabeled. As noted above, these differential reagents differ in molecular mass, but do not differ in retention properties regarding the separation method used (e.g., chromatography) and the mass spectrometry methods used will not detect different ionization and detection properties. Thus, these differential reagents differ either in their isotope composition (i.e., they are isotopical reagents) or they differ structurally by a rather small fragment which change does not alter the properties stated above (i.e., they are homologous reagents).

Differential chemical labeling can include esterification of C-termini, amidation of C-termini and/or acylation of N-termini. Esterification targets C-termini of peptides and carboxylic acid groups in amino acid side chains. Amidation targets C-termini of peptides and carboxylic acid groups in amino acid side chains. Amidation may require protection of amine groups first. Acylation targets N-termini of peptides and amino and hydroxy groups in amino acid side chains. Acylation may require protection of carboxylic groups first.

The skilled artisan will recognize that the chemical syntheses and differential chemical labeling of peptides and polypeptides (e.g., esterification, amidation, and acylation) used to practice the methods of the invention can be by a variety of procedures and methodologies, which are well described in the scientific and patent literature, e.g., Organic Syntheses Collective Volumes, Gilman et al. (Eds), John Wiley & Sons, Inc., NY; Venuti (1989) Pharm. Res. 6: 867-873; the Beilstein Handbook of Organic Chemistry (Beilstein Institut fuer Literatur der Organischen Chemie, Frankfurt, Germany); Beilstein online database and references obtainable therein; “Organic Chemistry,” Morrison & Boyd, 7th edition, 1999, Prentice-Hall, Upper Saddle River, N.J. The invention can be practiced in conjunction with any method or protocol known in the art, which are well described in the scientific and patent literature. For example, the esterification, amidation, and acylation reactions may be performed on the mixtures of peptides in a fashion similar to other reaction of these types already described in prior art, such as:

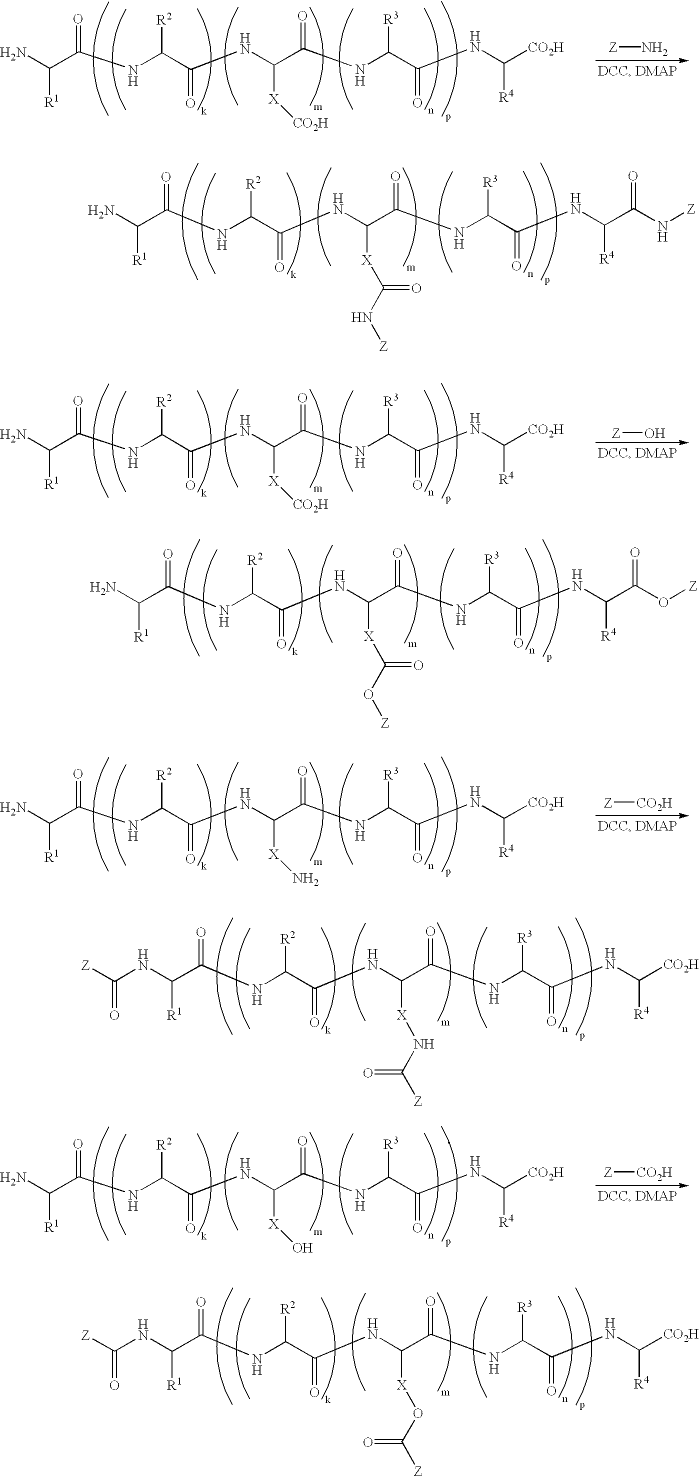

In alternative aspects, reagents comprise the general formulae:

Z^{A}OH and Z^{B}OH to esterify peptide C-terminals and/or Glu and Asp side chains;

Z^{A}NH₂/Z^{B}NH₂ to form amide bon d with peptide C-terminals and/or Glu and Asp side chains; or

Z^{A}CO₂H/Z^{B}CO₂H to form amide bond with peptide N-terminals and/or Lys and Arg side chains;

wherein Z^{A} and Z^{B} independently of one another can be R-Z¹-A¹-Z²-A²-Z³-A³-Z⁴-A⁴-, and Z¹, Z², Z³, and Z⁴ independently of one another can be selected from O, OC(O), OC(S), OC(O)O, OC(O)NR, OC(S)NR, OSiRR¹, S, SC(O), SC(S), SS, S(O), S(O₂), NR, NRR¹⁺, C(O), C(O)O, C(S), C(S)O, C(O)S, C(O)NR, C(S)NR, SiRR¹, (Si(RR¹)O)n, SnRR¹, Sn(RR¹)O, BR(OR¹), BRR¹, B(OR)(OR¹), OBR(OR¹), OBRR¹, OB(OR)(OR¹), or, Z¹, Z², Z³, and Z⁴ independently of one another may be absent, and R is an alkyl group; and, A¹, A², A³, and A⁴ independently of one another can be selected from (CRR¹)n, and R is an alkyl group. In alternative aspects, some single C—C bonds from (CRR¹)n may be replaced with double or triple bonds, in which case some groups R and R¹ will be absent, (CRR¹)n can be an o-arylene, an m-arylene, or a p-arylene with up to 6 substituents, carbocyclic, bicyclic, or tricyclic fragments with up to 8 atoms in the cycle with or without heteroatoms (O, N, S) and with or without substituents, or A¹, A², A³, and A⁴ independently of one another can be absent; R, R¹, independently from other R and R¹ in Z¹-Z⁴ and independently from other R and R¹ in A¹-A⁴, can be hydrogen, halogen or an alkyl group, such as an alkenyl, an alkynyl or an aryl group; n in Z¹-Z⁴, independent of n in A¹-A⁴, is an integer that can have value from 0 to about 51; 0 to about 41; 0 to about 31; 0 to about 21, 0 to about 11; 0 to about 6;

In alternative aspects, Z^{A} has the same structure as Z^{B}, but they have different isotope compositions. Any isotope may be used. In alternative aspects, if Z^{A} contains x number of protons, Z^{B} may contain y number of deuterons in the place of protons, and, correspondingly, x-y number of protons remaining; and/or if Z^{A} contains x number of borons-10, Z^{B} may contain y number of borons-11 in the place of borons-10, and, correspondingly, x-y number of borons-10 remaining; and/or if Z^{A} contains x number of carbons-12, Z^{B} may contain y number of carbons-13 in the place of carbons-12, and, correspondingly, x-y number of carbons-12 remaining; and/or if Z^{A} contains x number of nitrogens-14, Z^{B} may contain y number of nitrogens-15 in the place of nitrogens-14, and, correspondingly, x-y number of nitrogens-14 remaining; and/or if Z^{A} contains x number of sulfurs-32, Z^{B} may contain y number of sulfurs-34 in the place of sulfurs-32, and, correspondingly, x-y number of sulfurs-32 remaining; and so on for all elements which may be present and have different stable isotopes; x and y are whole numbers such that x is greater than y. In one aspect, x and y are between 1 and about 11, between 1 and about 21, between 1 and about 31, between 1 and about 41, between 1 and about 51.

In alternative aspects, reagent pairs/series comprise the general formulae:

CD₃(CD₂)ₙOH/CH₃(CH₂)ₙOH to esterify peptide C-terminals, where n=0, 1, 2, . . . , y; (delta mass=3+2n);

CD₃(CD₂)ₙNH₂/CH₃(CH₂)ₙNH₂ to form amide bond with peptide C-terminals where n=0, 1, 2, . . . , y (delta mass=3+2n);

D(CD₂)ₙCO₂H/H(CH₂)ₙCO₂H to form amide bond with peptide N-terminals, where n=0, 1, 2, . . . , y (delta mass=1+2n);

wherein y is an integer that can have value of about 51; about 41; about 31; about 21, about 11; about 6, or between about 5 and 51.

Other exemplary reagents can be presented by general formulae:

i. Z^{A}OH and Z^{B}OH to esterify peptide C-terminals;

Z^{A}NH₂/Z^{B}NH₂ to form an amide bond with peptide C-terminals;

Z^{A}CO₂H/Z^{B}CO₂H to form an amide bond with peptide N-terminals;

wherein Z^{A} and Z^{B} can be R-Z¹-A¹-Z²-A²-Z³-A³-Z⁴-A⁴-

and Z¹, Z², Z³, and Z⁴, independently of one another, can be selected from O, OC(O), OC(S), OC(O)O, OC(O)NR, OC(S)NR, OSiRR¹, S, SC(O), SC(S), SS, S(O), S(O₂), NR, NRR¹⁺, C(O), C(O)O, C(S), C(S)O, C(O)S, C(O)NR, C(S)NR, SiRR¹, (Si(RR¹)O)n, SnRR¹, Sn(RR¹)O, BR(OR¹), BRR¹, B(OR)(OR¹), OBR(OR¹), OBRR¹, or OB(OR)(OR¹); or, Z¹, Z², Z³, and Z⁴, independently of one another, can be absent, and, R is an alkyl group;

A¹, A², A³, and A⁴, independently of one another, can be a moiety comprising the general formulae (CRR¹)n. In alternative aspects, single C—C bonds in some (CRR¹)n groups may be replaced with double or triple bonds, in which case some groups R and R¹ will be absent, or (CRR¹)n can be an o-arylene, an m-arylene, or a p-arylene with up to 6 substituents, or a carbocyclic, a bicyclic, or a tricyclic fragments with up to 8 atoms in the cycle, with or without heteroatoms (e.g., O, N or S atoms), or, with or without substituents, or, A¹-A⁴ independently of one another may be absent;

In alternative aspects, R, R¹, independently from other R and R¹ in Z¹-Z⁴ and independently from other R and R¹ in A¹-A⁴, can be a hydrogen atom, a halogen or an alkyl group, such as an alkenyl, an alkynyl or an aryl group;

In alternative aspects, n in Z¹-Z⁴ is independent of n in A¹-A⁴ and is an integer that can have value of about 51; about 41; about 31; about 21, about 11; about 6.

In alternative aspects, Z^{A} has a similar structure to that of Z^{B}, but Z^{A} has x extra —CH₂— fragment(s) in one or more A¹-A⁴ fragments, and/or Z^{A} has x extra —CF₂— fragment(s) in one or more A¹-A⁴ fragments. Alternatively, Z^{A} can contain x number of protons and Z^{B} may contain y number of halogens in the place of protons. Alternatively, where Z^{A} contains x number of protons and Z^{B} contains y number of halogens, there are x-y number of protons remaining in one or more A¹-A⁴ fragments; and/or Z^{A} has x extra —O— fragment(s) in one or more A¹-A⁴ fragments; and/or Z^{A} has x extra —S— fragment( s) in one or more A¹-A⁴ fragments; and/or if Z^{A} contains x number of —O— fragment(s), Z^{B} may contain y number of —S— fragment(s) in the place of —O— fragment(s), and, correspondingly, x-y number of —O— fragment(s) remaining in one or more A¹-A⁴ fragments; and the like.

In alternative aspects, x and y are integers that can have value of between 1 about 51; of between 1 about 41; of between 1 about 31; of between 1 about 21, of between 1 about 11; of between 1 about 6, such that x is greater than y.

Exemplary homologous reagents pairs/series are

CH₃(CH₂)ₙOH/CH₃(CH₂)ₙ₊ₘOH to esterify peptide C-terminals, where n=0, 1, 2, . . . , y; m=1, 2, . . . , y (delta mass=14m)

CH₃(CH₂)ₙ NH₂/CH₃(CH₂)ₙ₊ₘNH₂ to form amide bond with peptide C-terminals, where n=0, 1, 2, . . . , y; m=1, 2, . . . , y (delta mass=14m)

H(CH₂)ₙCO₂H/H(CH₂)ₙ₊ₘCO₂H to form amide bond with peptide N-terminals, where n=0, 1, 2, . . . , y; m=1, 2, . . . , y (delta mass=14m)

wherein y is an integer that can have value of about 51; about 41; about 31; about 21, about 11; about 6, or between about 5 and 51.

Methods for Peptide/Protein Separation and Detection

The methods of the invention use chromatographic techniques to separate tagged polypeptides and peptides. In one aspect, a liquid chromatography is used, e.g., a multidimensional liquid chromatography. The chromatogram eluate is coupled to a mass spectrometer, such as a tandem mass spectrometry device (e.g., a “LC-LC-MS/MS” system). Any variation and equivalent thereof can be used to separate and detect peptides. LC-LC-MS/MS was first developed by Link A. and Yates J. R., as described, e.g., in (Link (1999) Nature Biotechnology 17:676-682; Link (2000) Electrophoresis 18, 1314-1334. In one aspect, the LC-LC-MS/MS technique is used; it is effective for complexed peptide separation and it is easily automated. LC-LC-MS/MS is commonly known by the acronym “MudPIT,” for “Multi-dimensional Protein Identification Technique.”

Variations and equivalents of LC-LC-MS/MS used in the methods of the invention include methodologies involving reversed phase columns coupled to either cation exchange columns (as described, e.g., by Opiteck (1997) Anal. Chem. 69:1518-1524; or, size exclusion columns (as described, e.g., by Opiteck (1997) Anal. Biochem. 258:349-361). In one aspect, an LC-LC-MS/MS technique uses a mixed bed microcapillary column containing strong cation exchange (SCX) and reversed phase (RPC) resins. Other exemplary alternatives include protein fractionation combined with one-dimensional LC-ESI MS/MS or peptide fractionation combined MALDI MS/MS.

Depending on the complexity or the property of the protein samples, any protein fractionation method, including size exclusion chromatography, ion exchange chromatography, reverse phase chromatography, or any of the possible affinity purifications, can be introduced prior to labeling and proteolysis. In some circumstances, use of several different methods may be necessary to identify all proteins or specific proteins in a sample.

Sequence Analysis and Quantification

Both quantity and sequence identity of the protein from which the modified peptide originated can be determined by a mass spectrometry device, such as a “multistage mass spectrometry” (MS). This can be achieved by the operation of the mass spectrometer in a dual mode in which it alternates in successive scans between measuring the relative quantities of peptides eluting from the capillary column and recording the sequence information of selected peptides. Peptides are quantified by measuring in the MS mode the relative signal intensities for pairs or series of peptide ions of identical sequence that are tagged differentially, which therefore differ in mass by the mass differential encoded within the differential labeling reagents.

Peptide sequence information can be automatically generated by selecting peptide ions of a particular mass-to-charge (m/z) ratio for collision-induced dissociation (CID) in the mass spectrometer operating in the tandem MS mode, as described, e.g., by Link (1997) Electrophoresis 18:1314-1334; Gygi (1999) Nature Biotechnol. 17:994-999; Gygi (1999) Cell Biol. 19:1720-1730.

The resulting tandem mass spectra can be correlated to sequence databases to identify the protein from which the sequenced peptide originated. Exemplary commercial available softwares include TURBO SEQUEST™ by Thermo Finnigan, San Jose, Calif.; MASSSCOT™ by Matrix Science, SONAR MS/MS™ by Proteometrics. Routine software modifications may be necessary for automated relative quantification.

Mass Spectrometry Devices

The methods of the invention can use mass spectrometry to identify and quantify differentially labeled peptides and polypeptides. Any mass spectrometry system can be used. In one aspect of the invention, combined mixtures of peptides are separated by a chromatography method comprising multidimensional liquid chromatography coupled to tandem mass spectrometry, or, “LC-LC-MS/MS,” see, e.g., Link (1999) Biotechnology 17:676-682; Link (1999) Electrophoresis 18:1314-1334. Exemplary, mass spectrometry devices include those incorporating matrix-assisted laser desorption-ionization-time-of-flight (MALDI-TOF) mass spectrometry (see, e.g., Isola (2001) Anal. Chem. 73:2126-2131; Van de Water (2000) Methods Mol. Biol. 146:453-459; Griffin (2000) Trends Biotechnol. 18:77-84; Ross (2000) Biotechniques 29:620-626, 628-629). The inherent high molecular weight resolution of MALDI-TOF MS conveys high specificity and good signal-to-noise ratio for performing accurate quantitation.

Use of mass spectrometry, including MALDI-TOF MS, and its use in detecting nucleic acid hybridization and in nucleic acid sequencing, is well known in the art, see, e.g., U.S. Pat. Nos. 6,258,538; 6,238,871; 6,238,869; 6,235,478; 6,232,066; 6,228,654; 6,225,450; 6,051,378; 6,043,031.

Fragmentation and Proteolytic Digestion

In practicing the methods of the invention, polypeptides can be fragmented, e.g., by proteolytic, i.e., enzymatic, digestion and/or other enzymatic reactions or physical fragmenting methodologies. The fragmentation can be done before and/or after reacting the peptides/polypeptides with the labeling reagents used in the methods of the invention. Methods for proteolytic cleavage of polypeptides are well known in the art, e.g., enzymes include trypsin (see, e.g., U.S. Pat. No. 6,177,268; 4,973,554), chymotrypsin (see, e.g., U.S. Pat. No. 4,695,458; 5,252,463), elastase (see, e.g., U.S. Pat. No. 4,071,410); subtilisin (see, e.g., U.S. Pat. No. 5,837,516) and the like.

In one aspect, a chimeric labeling reagent of the invention includes a cleavable linker. Exemplary cleavable linker sequences include, e.g., Factor Xa or enterokinase (Invitrogen, San Diego Calif.). Other purification facilitating domains can be used, such as metal chelating peptides, e.g., polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.).

Biological Samples

The methods are based on comparison of two or more samples of proteins, one of which can be considered as the standard sample and all others can be considered as samples under investigation. For example, in one aspect, the invention provides a method for quantifying changes in protein expression between at least two cellular states, such as, an activated cell versus a resting cell, a normal cell versus a cancerous cell, a stem cell versus a differentiated cell, an injured cell or infected cell versus an uninjured cell or uninfected cell; or, for defining the expressed proteins associated with a given cellular state.

Sample can be derived from any biological source, including cells from, e.g., bacteria, insects, yeast, mammals and the like. Cells can be harvested from any body fluid or tissue source, or, they can be in vitro cell lines or cell cultures.

Detection Devices and Methods

The devices and methods of the invention can also incorporate in whole or in part designs of detection devices as described, e.g., in U.S. Pat. Nos. 6,197,503; 6,197,498; 6,150,147; 6,083,763; 6,066,448; 6,045,996; 6,025,601; 5,599,695; 5,981,956; 5,698,089; 5,578,832; 5,632,957.

A number of aspects of the invention have been described. Nevertheless, it will be understood that various modifications may be made without departing from the spirit and scope of the invention.

### REFERENCES

Unless otherwise indicated, all references cited herein (supra and infra) are incorporated by reference in their entirety.

Gygi S P, Rist B, Gerber S A, Turecek F, Gelb M H, Aebersold R.: Quantitative analysis of complex protein mixtures using isotope-coded affinity tags. Nat Biotechnol 17(10):994-9 (October) 1999.

Hopkins M J, Sharp R, Macfarlane G T.: Age and disease related changes in intestinal bacterial populations assessed by cell culture, 16S rRNA abundance, and community cellular fatty acid profiles. Gut 48(2):198-205 (February) 2001.

Ritchie N J, Schutter M E, Dick R P, Myrold D D.: Use of length heterogeneity PCR and fatty acid methyl ester profiles to characterize microbial communities in soil. Appl Environ Microbiol 66(4):1668-75 (April) 2000.

Khan A A, Wang R F, Cao W W, Franklin W, Cerniglia C E.: Reclassification of a polycyclic aromatic hydrocarbon-metabolizing bacterium, Beijerinckia sp. strain B1, as Sphingomonas yanoikuyae by fatty acid analysis, protein pattern analysis, DNA-DNA hybridization, and 16S ribosomal DNA sequencing. Int J Syst Bacteriol 46(2):466-9 (April) 1996.

Peltroche-Llacsahuanga H, Schmidt S, Lutticken R, Haase G.: Discriminative power of fatty acid methyl ester (FAME) analysis using the microbial identification system (MIS) for Candida (Torulopsis) glabrata and Saccharomyces cerevisiae. Diagn Microbiol Infect Dis 38(4):213-21 (December) 2000.

S A Gerber et al.: Analysis of rates of multiple enzymes in cell lysates by electrospray ionization mass spectrometry. J. Am. Chem. Soc. 121:1102-3 1999.

David Goodlett discusses the latest in genomics—ICAT reagents

Written by: Marian Moser Jones

Dec. 20, 2000

WO0011208; Filed Aug. 25, 1999, Published Mar. 2, 2000. Aebersold R H, Gelb M H, Gygi, S P, Scott C R, Turecek F, Gerber S A, Rist B: Rapid quantitative analysis of proteins or protein function in complex mixtures.

WO9905221; Filed Jul. 27 1998, Published Feb. 4, 1999. Cummins W J, West R M, Smith J A: Cyanine Dyes.

U.S. Pat. No. 4,876,350; Filed Dec. 16, 1987, Issued Oct. 24, 1989. McGarrity J, Tenud L: Process for the production of (+) biotin.

U.S. Pat. No. 5,776,723; Filed Feb. 8, 1996, Issued Jul. 7, 1998. Herold C D, O'Hagan M: Rapid detection of mycobacterium tuberculosis.

U.S. Pat. No. 6,136,173; Filed Jun. 24, 1996, Issued Oct. 24, 2000. Anderson N L, Anderson N G, Goodman J: Automated system for two-dimensional electrophoresis.

U.S. Pat. No. 6,127,134; Filed Apr. 20, 1995, Issued Oct. 3, 2000. Minden J, Waggoner A: Difference gel electrophoresis using matched multiple dyes.

U.S. Pat. No. 6,064,754; Filed Dec. 1, 1997, Issued May 16, 2000. Parekh R B, Amess R, Bruce J A, Prime S B, Platt A E, Stoney R M: Computer-assisted methods and apparatus for identification and characterization of biomolecules in a biological sample.

U.S. Pat. No. 6,013,165; Filed May 22, 1998, Issued Jan. 11,2000. Wiktorowicz J E, Raysberg Y: Electrophoresis apparatus and method.

Ausubel F M, Brent R, Kingston R E, Moore D D, Seidman J G, Smith J A, Struhl K Editors.Current Protocols In Molecular Biology, Vol 2. John Wiley & Sons, Inc, ©2001, 10.21.4-10.21.6, 10.22.5-10.22.10, 10.22.14, 10.22.15-10.22.20.

Sambrook J, Russell D W Editors. Molecular Cloning A Laboratory Manual 3^{rd} ed. Cold Spring Harbor Laboratory Press, New York, ©2001, 18.3, 18.62, 18.66.

Alting-Mecs M A and Short J M: Polycos vectors: a system for packaging filamentous phage and phagemid vectors using lambda phage packaging extracts. Gene 137:1, 93-100, 1993.

Arkin A P and Youvan D C: An algorithm for protein engineering: simulations of recursive ensemble mutagenesis. Proc Natl Acad Sci USA 89(16):7811 -7815, (Aug. 15) 1992.

Arnold F H: Protein engineering for unusual environments. Current Opinion in Biotechnology 4(4):450-455, 1993.

Ausubel F M, et al Editors. Current Protocols in Molecular Biology, Vols. 1 and 2 and supplements. (a.k.a. “The Red Book”) Greene Publishing Assoc., Brooklyn, N.Y., ©1987.

Ausubel F M, et al Editors. Current Protocols in Molecular Biology, Vols. 1 and 2 and supplements. (a.k.a. “The Red Book”) Greene Publishing Assoc., Brooklyn, N.Y., ©1989.

Ausubel F M, et al Editors. Short Protocols in Molecular Biology: A Compendium of Methods from Current Protocols in Molecular Biology. Greene Publishing Assoc., Brooklyn, N.Y., ©1989.

Ausubel F M, et al Editors. Short Protocols in Molecular Biology: A Compendium of Methods from Current Protocols in Molecular Biology, 2^{nd} Edition. Greene Publishing Assoc., Brooklyn, N.Y., ©1992.

Barbas C F 3d, Bain J D, Hoekstra D M, Lemer R A: Semisynthetic combinatorial antibody libraries: a chemical solution to the diversity problem. Proc Natl Acad Sci USA 89(10):4457-4461, 1992.

Bardwell A J, Bardwell L, Johnson D K, Friedberg E C: Yeast DNA recombination and repair proteins Rad1 and Rad10 constitute a complex in vivo mediated by localized hydrophobic domains. Mol Microbiol 8(6):1177-1188, 1993.

Barret A J, et al., eds.: Enzyme Nomenclature: Recommendations of the Nomenclature Committee of the International Union of Biochemistry and Molecular Biology. San Diego: Academic Press, Inc., 1992.

Bartel P, Chien C T, Sternglanz R, Fields S: Elimination of false positives that arise in using the two-hybrid system. Biotechniques 14(6):920-924, 1993.

Beaudry A A and Joyce G F: Directed evolution of an RNA enzyme. Science 257(5070):635-641, 1992.

Berger and Kimmel, Methods in Enzymology, Volume 152, Guide to Molecular Cloning Techniques. Academic Press, Inc., San Diego, Calif., ©1987. (Cumulative Subject Index: Volumes 135-139, 141-167, 1990, 272 pp.)

Bevan M: Binary Agrobacterium vectors for plant transformation. Nucleic Acids Research 12(22):8711-21, 1984.

Biocca S, Pierandrei-Amaldi P, Cattaneo A: Intracellular expression of anti-p21 ras single chain Fv fragments inhibits meiotic maturation of xenopus oocytes. Biochem Biophys Res Commun 197(2):422-427, 1993.

Bird et al. Plant Mol Biol 11:651, 1988.

Bogerd H P, Fridell R A, Blair W S, Cullen B R: Genetic evidence that the Tat proteins of human immunodeficiency virus types 1 and 2 can multimerize in the eukaryotic cell nucleus. J Virol 67(8):5030-5034, 1993.

Boyce COL, ed.: Novo's Handbook of Practical Biotechnology. 2^{nd} ed. Bagsvaerd, Denmark, 1986.

Brederode F T, Koper-Zawrthoff E C, Bol J F: Complete nucleotide sequence of alfalfa mosaic virus RNA 4. Nucleic Acids Research 8(10):2213-23, 1980.

Breitling F, Dubel S, Seehaus T, Klewinghaus I, Little M: A surface expression vector for antibody screening. Gene 104(2):147-153, 1991.

Brown N L, Smith M: Cleavage specificity of the restriction endonuclease isolated from Haemophilus gallinarum (Hga I). Proc Natl Acad Sci USA 74(8):3213-6, (August) 1977.

Burton D R, Barbas CF 3d, Persson M A, Koenig S, Chanock R M, Lemer R A: A large array of human monoclonal antibodies to type 1 human immunodeficiency virus from combinatorial libraries of asymptomatic seropositive individuals. Proc Natl Acad Sci USA 88(22):10134-7, (Nov. 15) 1991.

Caldwell R C and Joyce G F: Randomization of genes by PCR mutagenesis. PCR Methods Appl 2(10):28-33, 1992.

Caton A J and Koprowski H: Influenze virus hemagglutinin-specific antibodies isolated from a combinatorial expression library are closely related to the immune response of the donor. Proc Natl Acad Sci USA 87(16):6450-6454, 1990.

Chakraborty T, Martin J F, Olson E N: Analysis of the oligomerization of myogenin and E2A products in vivo using a two-hybrid assay system. J Biol Chem 267(25):17498-501, 1992.

Chang C N, Landolfi N F, Queen C: Expression of antibody Fab domains on bacteriophage surfaces. Potential use for antibody selection. J Immunol 147(10):3610-4, (Nov. 15) 1991.

Chaudhary V K, Batra J K, Gallo M G, Willingham M C, FitzGerald D J, Pastan I: A rapid method of cloning functional variable-region antibody genes in Escherichia coli as single-chain immunotoxins. Proc Natl Acad Sci USA 87(3):1066-1070, 1990.

Chien C T, Bartel P L, Stemglanz R, Fields S: The two-hybrid system: a method to identify and clone genes for proteins that interact with a protein of interest. Proc Natl Acad Sci USA 88(21):9578-9582, 1991.

Chiswell D J, McCafferty J: Phage antibodies: will new ‘coliclonal’ antibodies replace monoclonal antibodies? Trends Biotechnol 10(3):80-84, 1992.

Chothia C and Lesk A M: Canonical structures for the hypervariable regions of immunoglobulins. J Mol Biol 196)4):901-917, 1987.

Chothia C, Lesk A M, Tramontano A, Levitt M, Smith-Gill S J, Air G, Sheriff S, Padlan E A, Davies D, Tulip W R, et al: Conformations of immunoglobulin hypervariable regions. Nature 342(6252):877-883, 1989.

Clackson T, Hoogenboom H R, Griffiths A D, Winter G: Making antibody fragments using phage display libraries. Nature 352(6336):624-628, 1991.

Conrad M, Topal M D: DNA and spermidine provide a switch mechanism to regulate the activity of restriction enzyme Nae I. Proc Natl Acad Sci U S A 86(24):9707-1 1, (December) 1989.

Coruzzi G, Broglie R, Edwards C, Chua N H: Tissue-specific and light-regulated expression of a pea nuclear gene encoding the small subunit of ribulose-1,5-bisophosphate carboxylase. EMBO J 3(8):1671-9, 1984.

Dasmahapatra B, DiDomenico B, Dwyer S, Ma J, Sadowski I, Schwartz J: A genetic system for studying the activity of a proteolytic enzyme. Proc Natl Acad Sci USA 89(9):4159-4162, 1992.

Davis L G, Dibner M D, Battey J F. Basic Methods in Molecular Biology. Elsevier, New York, N.Y., ©1986.

Delegrave S and Youvan D C. Biotechnology Research 11:1548-1552, 1993.

DeLong E F, Wu K Y, Prezelin B B, Jovine R V: High abundance of Archaea in Antarctic marine picoplankton. Nature 371(6499):695-697, 1994.

Deng S J, MacKenzie C R, Sadowska J, Michniewicz J, Young N M, Bundle Dr, Narang S A: Selection of antibody single-chain variable fragments with improved carbohydrate binding by phage display. J Biol Chem 269(13):9533-9538, 1994.

Drauz K, Waldman H, eds.: Enzyme Catalysis in Organic Synthesis: A Comprehensive Handbook. Vol. 1. New York: VCH Publishers, 1995.

Drauz K, Waldman H, eds.: Enzyme Catalysis in Organic Synthesis: A Comprehensive Handbook. Vol. 2. New York: VCH Publishers, 1995.

Duan L, Bagasra O, Laughlin M A, Oakes J W, Pomerantz R J: Potent inhibition of human immunodeficiency virus type 1 replication by an intracellular anti-Rev single-chain antibody. Proc Natl Acad Sci USA 91(11):5075-5079, 1994.

Durfee T, Becherer K, Chen P L, Yeh S H, Yang Y, Kilburn A E, Lee W H, Elledge S J: The retinoblastoma protein associates with the protein phosphatase type 1 catalytic subunit. Genes Dev 7(4):555-569, 1993.

Ellington A D and Szostak J W: In vitro selection of RNA molecules that bind specific ligands. Nature 346(6287):818-822, 1990.

Fields S and Song 0: A novel genetic system to detect protein-protein interactions. Nature 340(6230):245-246, 1989.

Firek S, Draper J, Owen M R, Gandecha A, Cockburn B, Whitelam G C: Secretion of a functional single-chain Fv protein in transgenic tobacco plants and cell suspension cultures. Plant Mol Biol 23(4):861-870, 1993.

Forsblom S, Rigler R, Ehrenberg M, Philipson L: Kinetic studies on the cleavage of adenovirus DNA by restriction endonuclease Eco RI. Nucleic Acids Res 3(12):3255-69, (December) 1976.

Foster G D, Taylor S C, eds.: Plant Virology Protocols: From Virus Isolation to Transgenic Resistance. Methods in Molecular Biology, Vol. 81. New Jersey: Humana Press Inc., 1998.

Franks F, ed.: Protein Biotechnology: Isolation, Characterization, and Stabilization. New Jersey: Humana Press Inc., 1993.

Germino F J, Wang Z X, Weissman S M: Screening for in vivo protein-protein interactions. Proc Natl Acad Sci USA 90(3):933-937, 1993.

Gingeras T R, Brooks J E: Cloned restriction/modification system from Pseudomonas aeruginosa. Proc Natl Acad Sci USA 80(2):402-6, (January) 1983.

Gluzman Y: SV40-transformed simian cells support the replication of early SV40 mutants. Cell 23(1):175-182, 1981.

Godfrey T, West S, eds.: Industrial Enzymology. 2^{nd} ed. London: Macmillan Press Ltd, 1996.

Gottschalk G: Bacterial Metabolism. 2^{nd} ed. New York: Springer-Verlag Inc., 1986.

Gresshoff P M, ed.: Technology Transfer of Plant Biotechnology. Current Topics in Plant Molecular Biology. Boca Raton: CRC Press, 1997.

Griffin H G, Griffin A M, eds.: PCR Technology: Currrent Innovations. Boca Raton: CRC Press, Inc., 1994.

Gruber M, Schodin B A, Wilson E R, Kranz D M: Efficient tumor cell lysis mediated by a bispecific single chain antibody expressed in Escherichia coli. J Immunol 152(11):5368-5374, 1994.

Guarente L: Strategies for the identification of interacting proteins. Proc Natl Acad Sci USA 90(5):1639-1641, 1993.

Guilley H, Dudley R K, Jonard G, Balazs E, Richards K E: Transcription of Cauliflower mosaic virus DNA: detection of promoter sequences, and characterization of transcripts. Cell 30(3):763-73, 1982.

Hansen G, Chilton M D: Lessons in gene transfer to plants by a gifted microbe. Curr Top Microbiol Immunol 240:21-57, 1999.

Hardy C F, Sussel L, Shore D: A RAP1-interacting protein involved in transcriptional silencing and telomere length regulation. Genes Dev 6(5):801-814, 1992.

Hartmann H T, et al.: Plant Propagation: Principles and Practices. 6^{th} ed. New Jersey: Prentice Hall, Inc., 1997.

Hawkins R E and Winter G: Cell selection strategies for making antibodies from variable gene libraries: trapping the memory pool. Eur J Immunol 22(3):867-870, 1992.

Holvoet P, Laroche Y, Lijnen H R, Van Hoef B, Brouwers E, De Cock F, Lauwereys M, Gansemans Y, Collen D: Biochemical characterization of single-chain chimeric plasminogen activators consisting of a single-chain Fv fragment of a fibrin-specific antibody and single-chain urokinase. Eur J Biochem 210(3):945-952, 1992.

Honjo T, Alt F W, Rabbitts T H (eds): Immunoglobulin genes. Academic Press: San Diego, Calif., pp. 361-368, 01989.

Hoogenboom H R, Griffiths A D, Johnson K S, Chiswell D J, Judson P, Winter G: Multi-subunit proteins on the surface of filamentous phage: methodologies for displaying antibody (Fab) heavy and light chains. Nucleic Acids Res 19(15):4133-4137, 1991.

Huse W D, Sastry L, Iverson S A, Kang A S, Alting-Mees M, Burton D R, Benkovic S J, Lemer R A: Generation of a large combinatorial library of the immunoglobulin repertoire in phage lambda. Science 246(4935):1275-1281, 1989.

Huston J S, Levinson D, Mudgett-Hunter M, Tai M S, Novotney J, Margolies M N, Ridge R J, Bruccoleri R E, Haber E, Crea R, et al: Protein engineering of antibody binding sites: recovery of specific activity in an anti-digoxin single-chain Fv analogue produced in Escherichia coli. Proc Natl Acad Sci USA 85(16):5879-5883, 1988.

Ivan Lefkovits, Editor. Immunology methods manual: the comprehensive sourcebook of techniques. Academic Press, San Diego, ©1997.

Iwabuchi K, Li B, Bartel P, Fields S: Use of the two-hybrid system to identify the domain of p53 involved in oligomerization. Oncogene 8(6):1693-1696, 1993.

Jackson A L, Pahl P M, Harrison K, Rosamond J, Sclafani R A: Cell cycle regulation of the yeast Cdc7 protein kinase by association with the Dbf4 protein. Mol Cell Biol 13(5):2899-2908, 1993.

Johnson S and Bird R E: Methods Enzymol 203:88, 1991.

Kabat et al: Sequences of Proteins of Immunological Interest, 4th Ed. U.S. Department of Health and Human Services, Bethesda, Md. (1987)

Kang A S, Barbas C F, Janda K D, Benkovic S J, Lemer R A: Linkage of recognition and replication functions by assembling combinatorial antibody Fab libraries along phage surfaces. Proc Natl Acad Sci USA 88(10):4363-4366, 1991.

Kettleborough C A, Ansell K H, Allen R W, Rosell-Vives E, Gussow D H, Bendig M M: Isolation of tumor cell-specific single-chain Fv from immunized mice using phage-antibody libraries and the re-construction of whole antibodies from these antibody fragments. Eur J Immunol 24(4):952-958, 1994.

Kruger D H, Barcak G J, Reuter M, Smith H O: EcoRII can be activated to cleave refractory DNA recognition sites. Nucleic Acids Res 16(9):3997-4008, (May 11) 1988.

Lalo D, Carles C, Sentenac A, Thuriaux P: Interactions between three common subunits of yeast RNA polymerases I and III. Proc Natl Acad Sci USA 90(12):5524-5528, 1993.

Laskowski M Sr: Purification and properties of venom phosphodiesterase. Methods Enzymol 65(1):276-84, 1980.

Lefkovits I and Pernis B, Editors. Immunological Methods, Vols. I and II. Academic Press, New York, N.Y. Also Vol. III published in Orlando and Vol. IV published in San Diego. ©1979-.

Lerner R A, Kang A S, Bain J D, Burton D R, Barbas CF 3d: Antibodies without immunization. Science 258(5086):1313-1314, 1992.

Leung, D. W., et al, Technique, 1:11-15, 1989.

Li B and Fields S: Identification of mutations in p53 that affect its binding to SV40 large T antigen by using the yeast two-hybrid system. FASEB J 7(10):957-963, 1993.

Lilley G G, Doelzal O, Hillyard C J, Bernard C, Hudson P J: Recombinant single-chain antibody peptide conjugates expressed in Escherichia coli for the rapid diagnosis of HIV. J Immunol Methods 171(2):211-226, 1994.

Lowman H B, Bass S H, Simpson N, Wells J A: Selecting high-affinity binding proteins by monovalent phage display. Biochemistry 30(45):10832-10838, 1991.

Luban J, Bossolt K L, Franke E K, Kalpana G V, Goff S P: Human immunodeficiency virus type 1 Gag protein binds to cyclophilins A and B. Cell 73(6):1067-1078, 1993.

Madura K, Dohmen R J, Varshavsky A: N-recognin/Ubc2 interactions in the N-end rule pathway. J Biol Chem 268(16):12046-54, (Jun 5) 1993.

Marks J D, Griffiths Ad, Malmqvist M, Clackson T P, Bye J M, Winter G: By-passing immunization: building high affinity human antibodies by chain shuffling. Biotechnology (NY) 10(7):779-783, 1992.

Marks J D, Hoogenboom H R, Bonnert T P, McCafferty J, Griffiths A D, Winter G: By-passing immunization. Human antibodies from V-gene libraries displayed on phage. J Mol Biol 222(3):581-597, 1991.

Marks J D, Hoogenboom H R, Griffiths A D, Winter G: Molecular evolution of proteins on filamentous phage. Mimicking the strategy of the immune system. J Biol Chem 267(23):16007-16010, 1992.

Maxam A M, Gilbert W: Sequencing end-labeled DNA with base-specific chemical cleavages. Methods Enzymol 65(l):499-560, 1980.

McCafferty J, Griffiths A D, Winter G, Chiswell D J: Phage antibodies: filamentous phage displaying antibody variable domains. Nature 348(6301):552-554, 1990.

Method of DNA sequencing.

Miller J H. A Short Course in Bacterial Genetics: A Laboratory Manual and Handbook for Escherichia coli and Related Bacteria (see inclusively p. 445). Cold Spring Harbor Laboratory Press, Plainview, N.Y., ©1992.

Milne G T and Weaver D T: Dominant negative alleles of RAD52 reveal a DNA repair/recombination complex including Rad51 and Rad52. Genes Dev 7(9):1755-1765, 1993.

Mullinax R L, Gross E A, Amberg J R, Hay B N, Hogrefe H H, Kubtiz M M, Greener A, Alting-Mees M, Ardourel D, Short J M, et al: Identification of human antibody fragment clones specific for tetanus toxoid in a bacteriophage lambda immunoexpression library. Proc natl Acad Sci USA 87(20):8095-9099, 1990.

Nath K, Azzolina B A: in Gene Amplification and Analysis (ed. Chirikjian J G), vol. 1, p. 113, Elsevier North Holland, Inc., New York, N.Y., ©1981.

Needleman S B and Wunsch C D: A general method applicable to the search for similarities in the amino acid sequence of two proteins. J Mol Biol 48(3):443-453, 1970.

Nelson M, Christ C, Schildkraut I: Alteration of apparent restriction endonuclease recognition specificities by DNA methylases. Nucleic Acids Res 12(13):5165-73, 1984 (July 11).

Nicholls P J, Johnson V G, Andrew S M, Hoogenboom H R, Raus J C, Youle R J: Characterization of single-chain antibody (sFv)-toxin fusion proteins produced in vitro in rabbit reticulocyte lysate. J Biol Chem 268(7):5302-5308, 1993.

Oller A R, Vanden Broek W, Conrad M, Topal M D: Ability of DNA and spermidine to affect the activity of restriction endonucleases from several bacterial species. Biochemistry 30(9):2543-9, (Mar. 5) 1991.

Owen MRL, Pen J: Transgenic Plants: A Production System for Industrial and Pharmaceutical Proteins. Chichester: John Wiley & Sons, 1996.

Owens R J and Young R J: The genetic engineering of monoclonal antibodies. J Immunol Methods 168(2):149-165, 1994.

Pearson W R and Lipman D J: Improved tools for biological sequence comparison. Proc Natl Acad Sci USA 85(8):2444-2448, 1988.

Pein C D, Reuter M, Meisel A, Cech D, Kruger D H: Activation of restriction endonuclease EcoRII does not depend on the cleavage of stimulator DNA. Nucleic Acids Res 19(19):5139-42, (Oct. 11) 1991.

Persson M A, Caothien R H, Burton D R: Generation of diverse high-affinity human monoclonal antibodies by repertoire cloning. Proc Natl Acad Sci USA 88(6):2432-2436, 1991.

Perun T J, Propst C L, eds.: Computer-Aided Drug Design: Methods and Applications. New York: Marcel Dekker, Inc., 1989.

Qiang B Q, McClelland M, Poddar S, Spokauskas A, Nelson M: The apparent specificity of NotI (5′-GCGGCCGC-3′) is enhanced by M.FnuDII or M.BepI methyltransferases (5′-mCGCG-3′): cutting bacterial chromosomes into a few large pieces. Gene 88(1):101-5, (Mar. 30) 1990.

Queen C, Foster J, Stauber C, Stafford J: Cell-type specific regulation of a kappa immunoglobulin gene by promoter and enhance elements. Immunol Rev 89:49-68, 1986.

Raleigh E A, Wilson G: Escherichia coli K-12 restricts DNA containing 5-methylcytosine. Proc Natl Acad Sci USA 83(23):9070-4, (December) 1986.

Reidhaar-Olson J F and Sauer R T: Combinatorial cassette mutagenesis as a probe of the informational content of protein sequences. Science 241(4861):53-57, 1988.

Riechmann L and Weill M: Phage display and selection of a site-directed randomized single-chain antibody Fv fragment for its affinity improvement. Biochemistry 32(34):8848-8855, 1993.

Roberts R J, Macelis D: REBASE—restriction enzymes and methylases. Nucleic Acids Res 24(1):223-35, (Jan. 1) 1996.

Ryan A J, Royal C L, Hutchinson J, Shaw C H: Genomic sequence of a 12S seed storage protein from oilseed rape (Brassica napus c.v. jet neuf). Nucl Acids Res 17(9):3584, 1989.

Sambrook J. Fritsch E F, Maniatis T. Molecular Cloning: A Laboratory Manual. Cold Spring Harbor Laboratory Press, Cold Spring Harbor, N.Y., ©1982.

Sambrook J, Fritsch E F, Maniatis T. Molecular Cloning: A Laboratory Manual. Second Edition. Cold Spring Harbor Laboratory Press, Cold Spring Harbor, N.Y., ©1989.

Scopes R K. Protein Purification: Principles and Practice. Springer-Verlag, New York, N.Y., ©1982.

Segel I H: Enzyme Kinetics: Behavior and Analysis of Rapid Equilibrium and Steady-State Enzyme Systems. New York: John Wiley & Sons, Inc., 1993.

Silver S C and Hunt S W 3d: Techniques for cloning cDNAs encoding interactive transcriptional regulatory proteins. Mol Biol Rep 17(3): 155-165, 1993.

Smith T F, Waterman M S, Fitch W M: Comparative biosequence metrics. J Mol Evol S18(1):38-46, 1981.

Smith T F, Waterman M S. Adv Appl Math 2: 482-end of article, 1981.

Smith T F, Waterman M S: Identification of common molecular subsequences. J Mol Biol 147(1):195-7, (Mar. 25) 1981.

Smith T F, Waterman M S: Overlapping genes and information theory. J Theor Biol 91(2):379-80, (Jul. 21) 1981.

Staudinger J, Perry M, Elledge S J, Olson E N: Interactions among vertebrate helix-loop-helix proteins in yeast using the two-hybrid system. J Biol Chem 268(7):4608-4611, 1993.

Stemmer W P, Morris S K, Wilson B S: Selection of an active single chain Fv antibody from a protein linker library prepared by enzymatic inverse PCR. Biotechniques 14(2):256-265, 1993.

Stemmer W P: DNA shuffling by random fragmentation and reassembly: in vitro recombination for molecular evolution. Proc Natl Acad Sci USA 91(22):10747-10751, 1994.

Sun D, Hurley L H: Effect of the (+)-CC-1065-(N3-adenine)DNA adduct on in vitro DNA synthesis mediated by Escherichia coli DNA polymerase. Biochemistry 31:10, 2822-9, (Mar. 17) 1992,

Tague B W, Dickinson C D, Chrispeels M J: A short domain of the plant vacuolar protein phytohemagglutinin targets invertase to the yeast vacuole. Plant Cell 2(6):533-46, (June) 1990.

Takahashi N, Kobayashi I: Evidence for the double-strand break repair model of bacteriophage lambda recombination. Proc Natl Acad Sci USA 87(7):2790-4, (April) 1990.

Thiesen H J and Bach C: Target Detection Assay (TDA): a versatile procedure to determine DNA binding sites as demonstrated on SP1 protein. Nucleic Acids Res 18(11):3203-3209, 1990.

Thomas M, Davis R W: Studies on the cleavage of bacteriophage lambda DNA with EcoRI Restriction endonuclease. J Mol Biol 91(3):315-28, (Jan. 25) 1975.

Tingey S V, Walker E L, Corruzzi G M: Glutamine synthetase genes of pea encode distinct polypeptides which are differentially expressed in leaves, roots and nodules. EMBO J 6(1):1-9, 1987.

Topal M D, Thresher R J, Conrad M, Griffith J: Nael endonuclease binding to pBR322 DNA induces looping. Biochemistry 30(7):2006-10, (Feb. 19) 1991.

Tramontano A, Chothia C, Lesk A M: Framework residue 71 is a major determinant of the position and conformation of the second hypervariable region in the VH domains of immunoglobulins. J Mol Biol 215(1):175-182, 1990.

Tuerk C and Gold L: Systematic evolution of ligands by exponential enrichment: RNA ligands to bacteriophage T4 DNA polymerase. Science 249(4968):505-510, 1990.

U.S. Pat. No. 4,683,195; Filed Feb. 7, 1986, Issued Jul. 28. 1987. Mullis K B, Erlich H A, Arnheim N, Horn G T, Saiki R K, Scharf S J: Process for Amplifying, Detecting, and/or Cloning Nucleic Acid Sequences.

U.S. Pat. No. 4,683,202; Filed Oct. 25, 1985, Issued Jul. 28, 1987. Mullis K B: Process for Amplifying Nucleic Acid Sequences.

U.S. Pat. No. 4,704,362; Filed Nov. 5, 1979, Issued Nov. 3, 1987. Itakura K, Riggs A D: Recombinant Cloning Vehicle Microbial Polypeptide Expression.

U.S. Pat. No. 4,713,337; Filed Jan. 3, 1985, Issued Dec. 15, 1987. Jasin M, Schimmel P R: Method for deletion of a gene from a bacteria.

U.S. Pat. No. 4,732,856; Filed Apr. 3, 1984, Issued Mar. 22, 1988. Federoff N V: Transposable elements and process for using same.

U.S. Pat. No. 4,963,487; Filed Sep. 14, 1987, Issued Jan. 16, 1990. Schimmel P R: Method for deletion of a gene from a bacteria.

U.S. Pat. No. 5,354,656; Filed Oct. 2, 1989, Issued Oct. 11, 1994. Sorge, Joseph A.; Huse, William D.:

U.S. Pat. No. 5,385,835; Filed May 19, 1994, Issued Jan. 31, 1995. Helentjaris, Timothy; Nienhuis, James: Identification and localization and introgression into plants of desired multigenic traits.

U.S. Pat. No. 5,453,247; Filed Nov. 23, 1993, Issued Sep. 26, 1995. Beavis, Ronald C.; Chait, Brian T.: Instrument and method for the sequencing of genome.

U.S. Pat. No. 5,604,100; Filed Jul. 19, 1995, Issued Feb. 18, 1997. Perlin, Mark W.: Method and system for sequencing genomes.

U.S. Pat. No. 5,670,321; Filed May 10, 1995, Issued Sep. 23, 1997. Kimmel, Bruce E.; Ellis, Michael ; Ruddy, David: Efficient method to conduct large-scale genome sequencing.

U.S. Pat. No. 5,925,808; Filed Dec. 19, 1997, Issued Jul. 20, 1999. Oliver, Melvin John; Quisenberry, Jerry Edwin; Trolinder, Norma Lee Glover; Keim, Don Lee: Control Of Plant Gene Expression.

U.S. Pat. No. 5,953,727; Filed Mar. 6, 1997, Issued Sep. 14, 1999. Maslyn, Timothy J.; Au-Young, Janice; Hillman, Jennifer L.; Hibbert, Harold; Akerblom, Ingrid E.; Cheng, Rachel J.; Tang, Yuanhua T.: Project-based full-length biomolecular sequence database.

U.S. Pat. No. 5,965,443; Filed Sep. 9, 1996, Issued Oct. 12, 1999. Reznikoff W S, Goryshin I Y: System for in vitro transposition.

U.S. Pat. No. 5,981,177; Filed Jan. 25, 1995, Issued Nov. 9, 1999. Demirjian D C, Casadaban M J, Weber M, Gaines G L: Protein fusion method and constructs.

U.S. Pat. No. 5,994,058; Filed Mar. 20, 1995, Issued Nov. 30, 1999. Senapathy, Periannan:Method For Contiguous Genome Sequencing.

U.S. Pat. No. 6,023,659; Filed Mar. 6, 1997, Issued Feb. 8, 2000. Seilhamer, Jeffrey J.; Akerblom, Ingrid E.; Altus, Christina M.; Klingler, Tod M.; Russo, Frank; Au-Young, Janice; Hillman, Jennifer L.; Maslyn, Timothy J.: Database System Employing Protein Function Hierarchies For Viewing Biomolecular Sequence Data.

van de Poll M L, Lafleur M V, van Gog F, Vrieling H, Meerman J H: N-acetylated and deacetylated 4′-fluoro-4-aminobiphenyl and 4-aminobiphenyl adducts differ in their ability to inhibit DNA replication of single-stranded M13 in vitro and of single-stranded phi X174 in Escherichia coli. Carcinogenesis 13(5):75 1-8, (May) 1992.

Vojtek A B, Hollenberg S M, Cooper J A: Mammalian Ras interacts directly with the serine/threonine kinase Raf. Cell 74(1):205-214, 1993.

Wenzler H, Mignery G, Fisher L, Park W: Sucrose-regulated expression of a chimeric potato tuber gene in leaves of transgenic tobacco plants. Plant Mol Biol 13(4):347-54, 1989.

White J S, White D C: Source Book of Enzymes. Boca Raton: CRC Press, 1997.

Williams and Barclay, in Immunoglobulin Genes, The Immunoglobulin Gene Superfamily

Winnacker E L. From Genes to Clones: Introduction to Gene Technology, VCH Publishers, New York, N.Y., ©1987.

Winter G and Milstein C: Man-made antibodies. Nature 349(6307):293-299, 1991.

WO 00/04190; Filed Jul. 15, 1999, Published Jan. 27, 2000. Del Cardayre S, Tobin M, Stemmer W P, Ness J E, Minshull J, Patten P A, Subramanian V, Castle L A, Krebber C M, Bass S, Zhang Y, Cox T, Huisman G, Yuan L, Affholter J A: Evolution of whole cells and organisms by recursive sequence recombination.

WO 00/09755; Filed Aug. 12, 1999, Published Feb. 24, 2000. Zarling D, Reddy G, Pati S: Domain specific gene evolution.

WO 88/08453; Filed Apr. 14, 1988, Published Nov. 3, 1988. Alakhov J B, Baranov, V I, Ovodov S J, Ryabova L A, Spirin A S: Method of Obtaining Polypeptides in Cell-Free Translation System.

WO 90/05785; Filed Nov. 15, 1989, Published May 31, 1990. Schultz P: Method for Site-Specifically Incorporating Unnatural Amino Acids into Proteins.

WO 90/07003; Filed Jan. 27, 1989, Published Jun. 28, 1990. Baranov V I, Morozov I J, Spirin A S: Method for Preparative Expression of Genes in a Cell-free System of Conjugated Transcription/translation.

WO 91/02076; Filed Jun. 14, 1990, Published Feb. 21, 1991. Baranov V I, Ryabova L A, Yarchuk O B, Spirin A S: Method for Obtaining Polypeptides in a Cell-free System.

WO 91/05058; Filed Oct. 5, 1989, Published Apr. 18, 1991. Kawasaki G: Cell-free Synthesis and Isolation of Novel Genes and Polypeptides.

WO 91/17271; Filed May 1, 1990, Published Nov. 14, 1991. Dower W J, Cwirla S E: Recombinant Library Screening Methods.

WO 91/18980; Filed May 13, 1991, Published Dec. 12, 1991. Devlin J J: Compositions and Methods for Indentifying Biologically Active Molecules.

WO91/19818;Filed Jun. 20, 1990, Published Dec. 26, 1991. Dower W J, Cwirla S E, Barrett R W: Peptide Library and Screening Systems.

WO 92/02536; Filed Aug. 1, 1991, Published Feb. 20, 1992. Gold L, Tuerk C: Systematic Polypeptide Evolution by Reverse Translation.

WO 92/03918; Filed Aug.28, 1991, Published Mar. 19, 1992. Lonberg N, Kay R M: Transgenic Non-human Animals Capable of Producing Heterologous Antibodies.

WO 92/05258; Filed Sep. 17, 1991, Published Apr. 2, 1992. Fincher G B: Gene Encoding Barley Enzyme.

WO 92/14843; Filed Feb. 21, 1992, Published Sep. 3, 1992. Toole J J, Griffin L C, Bock L C, Latham J A, Muenchau D D, Krawczyk S: Aptamers Specific for Biomolecules and Method of Making.

WO 93/08278; Filed Oct. 15, 1992, Published Apr. 29, 1993. Schatz P J, Cull M G, Miller J F, Stemmer W P: Peptide Library and Screening Method.

WO 93/12227; Filed Dec. 17, 1992, Published Jun. 24, 1993. Lonberg N, Kay R M: Transgenic Non-human Animals Capable of Producing Heterologous Antibodies.

WO 94/25585; Filed Apr.25, 1994, Published Nov. 10, 1994. Lonberg N, Kay R M: Transgenic Non-human Animals Capable of Producing Heterologous Antibodies.

WO 95/00530; Filed Jun. 6, 1994, Published Jan. 1, 1995. Fodor, Stephen, P., A.; Lipshutz, Robert, J.; Huang, Xiaohua; Jevons, Luis, Carlos: Hybridization and Sequencing of Nucleic Acids.

WO 96/21031; Filed Jun. 7, 1995, Published Jul. 11, 1996. Tricoli, David, M.; Carney, Kim, J.; Russell, Paul, F.; Quemada, Hector, D.; Mcmaster, J., Russell ; Reynolds, John, F.; Deng, Rosaline, Z.: Transgenic Plants Expressing DNA Constructs Containing A Plurality Of Genes To Impart Virus Resistance.

WO 96/27025; Filed Feb. 21, 1996, Published Sep. 6, 1996. Rabani, Ely, Michael:Device, Compounds, Algorithms, And Methods Of Molecular Characterization And Manipulation With Molecular Parallelism.

WO 97/17429; Filed Nov. 8, 1996, Published May 15, 1997. Oglevee-O'donovan, Wendy; Arteca, Richard, N.; Arteca, Jeannette; Stoots, Eleanor: Method For The Commercial Production Of Transgenic Plants.

WO 97/35966; Filed Mar. 20, 1997, Published Oct. 2, 1997. Minshull J, Stemmer W P: Methods and compositions for cellular and metabolic engineering.

WO 97/37041; Filed Mar. 18, 1997, Published Oct. 9, 1997. Köster, Hubert: DNA Sequencing By Mass Spectrometry.

WO 97/42348; Filed May 5, 1997, Published Nov. 13, 1997. Köster, Hubert ; Van Den Boom, Dirk; Ruppert, Andreas: Process For Direct Sequencing During Template Amplification.

WO 98/26407; Filed Dec. 11, 1997, Published Jun. 18, 1998. Sabatini, Cathryn, E.; Heath, Joe, Don; Covitz, Peter, A.; Klinger, Tod, M.; Russo, Frank, D. Berry, Stephanie, F.: Database And System For Storing, Comparing And Displaying Genomic Information.

WO 98/26408; Filed Dec. 11, 1997, Published Jun. 18, 1998. Sabatini, Cathryn, E.; Heath, Joe, Don; Covitz, Peter, A.; Klingler, Tod, M.; Russo, Frank, D. Berry, Stephanie, F.:Database And System For Determining, Storing And Displaying Gene Locus Information.

WO 98/31833; Filed Dec. 12, 1997, Published Jul. 23, 1998. Ju, Jingyue: Nucleic Acid Sequencing With Solid Phase Capturable Terminators.

WO 98/31834; Filed Dec. 12, 1997, Published Jul. 23, 1998. Ju, Jingyue: Sets Of Labeled Energy Transfer Fluorescent Primers And Their Use In Multi Component Analysis.

WO 98/31837; Filed Jan. 16, 1998, Published Jul. 23, 1998. Delcardayre S B, Tobin M B, Stemmer W P, Ness J E, Minshull J, Patten P: Evolution of whole cells and organisms by recursive sequence recombination.

WO 98/36085; Filed Feb. 13, 1998, Published Aug. 20, 1998. Sutliff, Thomas, D.; Rodriguez, Raymond, L.: Production Of Mature Proteins In Plants.

WO 98/37223; Filed Feb. 18, 1998, Published Aug. 27, 1998. Pang, Sheng-Zhi ; Gonsalves, Dennis; Jan, Fuh-Jyh: DNA Construct To Confer Multiple Traits On Plants.

WO 99/35494; Filed Jan. 8, 1999, Published Jul. 15, 1999. Tally F P, Tao J, Wendler P A, Connelly G, Gallant P L: Method for identifying validated target and assay combinations.

WO 99/37755; Filed Dec. 11, 1998, Published Jul. 29, 1999. Pati S, Zarling David, Lehman C W, Zeng H: The use of consensus sequences for targeted homologous gene isolation and recombination in gene families.

WO 99/49403; Filed Mar. 25, 1999, Published Sep. 30, 1999. Lincoln, Stephen, E.; Hodgson, David, M.; Spiro, Peter, A.; Russo, Frank, D.; Akerblom, Ingrid, E.; Hillman, Jennifer, L.; Jones, Anissa, Lee ; Bratcher, Shawn, Robert; Cohen, Howard, Jerome; Dufour, Gerard; Wood, Michael, Peter; Koleszar, Alexander, George Banville, Steven, C.: System And Methods For Analyzing Biomolecular Sequences.

WO95/11995; Filed Oct. 26, 1994, Published May 4, 1995. Chee M, Cronin M T, Fodor S P, Gingeras T R, Huang X C, Hubbell E A, Lipshutz R J, Lobban P E, Miyada C G, Morris M S, Shah N, Sheldon E L: Arrays Of Nucleic Acid Probes On Biological Chips.

Wong CH, Whitesides GM: Enzymes in Synthetic Organic Chemistry. Vol. 12. New York: Elsevier Science Publications, 1995.

Yang X, Hubbard E J, Carlson M: A protein kinase substrate identified by the two-hybrid system. Science 257(5070):680-2, (Jul. 31) 1992.

Gygi S P, Rist B, Gerber S A, Turecek F, Gelb M H, Aebersold R.: Quantitative analysis of complex protein mixtures using isotope-coded affinity tags. Nat Biotechnol 17(10):994-9 (October) 1999.

Hopkins M J, Sharp R, Macfarlane G T.: Age and disease related changes in intestinal bacterial populations assessed by cell culture, 16S rRNA abundance, and community cellular fatty acid profiles. Gut 48(2): 198-205 (February) 2001.

Ritchie N J, Schutter M E, Dick R P, Myrold D D.: Use of length heterogeneity PCR and fatty acid methyl ester profiles to characterize microbial communities in soil.Appl Environ Microbiol 66(4):1668-75 (April) 2000.

Khan A A, Wang R F, Cao W W, Franklin W, Cerniglia C E.: Reclassification of a polycyclic aromatic hydrocarbon-metabolizing bacterium, Beijerinckia sp. strain B1, as Sphingomonas yanoikuyae by fatty acid analysis, protein pattern analysis, DNA-DNA hybridization, and 16S ribosomal DNA sequencing. Int J Syst Bacteriol 46(2):466-9 (April) 1996.

Peltroche-Llacsahuanga H, Schmidt S, Lutticken R, Haase G.: Discriminative power of fatty acid methyl ester (FAME) analysis using the microbial identification system (MIS) for Candida (Torulopsis) glabrata and Saccharomyces cerevisiae. Diagn Microbiol Infect Dis 38(4):213-21 (December) 2000.

S A Gerber et al.: Analysis of rates of multiple enzymes in cell lysates by electrospray ionization mass spectrometry. J. Am. Chem. Soc. 121:1102-3 1999.

www.genomeweb.com

David Goodlett discusses the latest in genomics—ICAT reagents

Written by: Marian Moser Jones

Dec. 20, 2000

WO0011208; Filed Aug. 25, 1999, Published Mar. 2, 2000. Aebersold R H, Gelb M H, Gygi, S P, Scott C R, Turecek F, Gerber S A, Rist B: Rapid quantitative analysis of proteins or protein function in complex mixtures.

WO9905221; Filed Jul. 27, 1998, Published Feb. 4, 1999. Cummins W J, West R M, Smith J A: Cyanine Dyes.

U.S. Pat. No. 4,876,350; Filed Dec. 16, 1987, Issued Oct. 24, 1989. McGarrity J, Tenud L: Process for the production of (+) biotin.

U.S. Pat. No. 5,776,723; Filed Feb. 8, 1996, Issued Jul. 7, 1998. Herold C D, O'Hagan M: Rapid detection of mycobacterium tuberculosis.

U.S. Pat. No. 6,136,173; Filed Jun. 24, 1996, Issued Oct. 24, 2000. Anderson N L, Anderson N G, Goodman J: Automated system for two-dimensional electrophoresis.

U.S. Pat. No. 6,127,134; Filed Apr. 20, 1995, Issued Oct. 3, 2000. Minden J, Waggoner A: Difference gel electrophoresis using matched multiple dyes.

U.S. Pat. No. 6,064,754; Filed Dec. 1, 1997, Issued May 16, 2000. Parekh R B, Aness R, Bruce J A, Prime S B, Platt A E, Stoney R M: Computer-assisted methods and apparatus for identification and characterization of biomolecules in a biological sample.

U.S. Pat. No. 6,013,165; Filed May 22, 1998, Issued Jan. 11, 2000. Wiktorowicz J E, Raysberg Y: Electrophoresis apparatus and method.

Ausubel F M, Brent R, Kingston R E, Moore D D, Seidman J G, Smith J A, Struhl K Editors.Current Protocols In Molecular Biology, Vol 2. John Wiley & Sons, Inc, ©2001, 10.21.4-10.21.6, 10.22.5-10.22.10, 10.22.14, 10.22.15-10.22.20.

Sambrook J, Russell D W Editors. Molecular Cloning A Laboratory Manual 3^{rd} ed. Cold Spring Harbor Laboratory Press, New York, ©2001, 18.3, 18.62, 18.66.

Additional Methods for Differential Analysis

Protein Expression Profiling Using Selective Differential Labeling

The use of mass spectrometry to identify proteins whose sequences are present in either DNA or protein databases is well established and integral to the field of Proteomics. Protein and peptide mass can be determined at high accuracy by several mass spectrometric techniques. Peptide can be further fragmented in a tandem or ion trap mass spectrometer yielding sequence information of the peptide. Both types of mass information can be used to identify protein in a sequence database. One goal of Proteomics is to define the expressed proteins associated with a given cellular state and another is to quantify changes in protein expression between cellular states. One of the new methodologies that have a great impact on proteome research is known as isotope-coded affinity tag (ICAT) peptide labeling (17). The method is based on a newly synthesized class of chemical reagents (ICATs) used in combination with tandem mass spectrometry. The ICAT reagent contains a biotin affinity tag and a thiol specific reactive group, which are joined by a spacer domain which is available in two forms: regular and isotopically heavy, which includes eight deuterium atoms. First, a reduced protein mixture representing one cell state is derivatized with the isotopically light version of the ICAT reagent, while the corresponding reduced protein mixture representing a second cell state is derivatized with the isotopically heavy version of the ICAT reagent. Second, the labeled samples are combined and proteolytically digested to produce peptide fragments. Third, the tagged cysteine containing peptide fragments are isolated by avidin affinity chromatography. Finally, the isolated tagged peptides are separated and analyzed by microcapillary tandem mass spectrometry.

There are, however, limitations associated with their approach: (i) differential labeling reagents relied on stable isotopes which is expensive and not very flexible to multiplex differential labeling; (ii) The moieties attached to the original peptides are approximately 500 Dalton heavy, which is heavier than some peptides and is likely to affect peptide ionization and fragmentation process; (iii) Some bonds in the labeling reagent are week compared to the amide bond, which might complicate the MS/MS spectrum, (iv) Protein expression profiling is limited to duplex comparison; (v) The affinity interaction between biotin and avidin is too strong to release the immobilized peptide efficiently.

In one aspect, this present invention provides a method for simultaneous identification and quantification of expression levels of individual proteins carrying certain functional groups in their side chains. The proteins may be analyzed in complex mixtures. The method is based on comparison of two or more samples of proteins, one of which can be considered as the standard sample and all others can be considered as samples under investigation.

The samples of proteins are subjected to a sequence of manipulations including (i) proteolytic digestion into mixtures of peptides, (ii) treatment of the mixtures of peptides with chemical probes, (iii) washing away and discarding the unbound peptides from the mixtures, (iv) cleaving the chemical probes and the consequential release of the peptides still carrying parts of the chemical probes into solution. This sequence of manipulations may also include one or more auxiliary chemical and/or enzymatic modifications of functional groups in side chains and/or in the free termini of the proteins and/or peptides in order to achieve selective and the most favorable modification for the next steps in the protocol. The auxiliary modifications may be performed between any steps of the main sequence.

The core structure of the chemical probe consists of (i) a solid support, (ii) a spacer, (iii) a cleavable moiety, (iv) a differential mass labeling unit, and (v) a reactive group. The chemical probes perform three functions: (i) they attach peptides carrying specific functional groups in their side chains and/or termini to a solid support by forming covalent chemical bonds to the reactive group of the probe, (ii) they provide means for selective cleavage of the attached peptide from the solid support such that a part of the probe still remains attached to the peptide, and (iii) they serve as differential labeling reagents.

Differential labeling results from attaching of chemical moieties of different mass but of similar properties to a protein or a peptide such that peptides with the same sequence but with different labels are eluted together in the separation procedure and their ionization and detection properties regarding mass spectrometrical analysis are very similar. The differential mass labeling unit remains covalently bound to the peptide after it is cleaved from the solid support part of the probe. Signals corresponding to peptides with the same sequence but marked with differential mass labels are assigned to different original protein samples.

The auxiliary chemical and/or enzymatic modification can be used to introduce additional differential mass labels into the peptides. The reactive group on the chemical probe may be activated or modified by a bridging reagent prior to a reaction with mixtures of peptides. Such activation or modification provides for a greater flexibility in design of the chemical probe since the same core structure of a chemical probe may be tuned to increase reactivity and/or selectivity towards different functional groups in side chains and/or in termini of the peptides.

After being cleaved from the solid support part of the chemical probe, the differentially labeled peptide mixtures are combined, subjected to multidimensional chromatographic separation, and analyzed by mass spectrometry methods. Mass spectrometry data is processed by special software, which allows for determination and tracing the composition and sequence of peptides in the mixture to identification of the original proteins and their quantification.

This approach can be used for duplex or potentially multiplex protein expression profiling. The complexity of the sample is simplified by targeting peptides containing particular amino acids, which selected by a reaction with chemical probes.

Alternative aspects of this invention include: (i) design of solid phase-based differential mass labeling reagents for selective peptide modification; (ii) design of various kinds of differential mass unit; (iii) combination of differential mass probes with various bridge reagent to target certain amino acid specifically; (iv) multiplex analysis; (v) combination of proteolytic digestion and chemical and/or enzymatic modifications in side chains and/or in termini of proteins and peptides in order to achieve selective and the most favorable modifications for the next steps in the protocol; (vi) combination of differential chemical labeling with MudPIT, and possible all other protein/peptide separation or purification technologies if necessary.

One aspect of this invention provides reagents and procedures for quantification of protein expression using combination of selective differential peptides labeling, and LC MS/MS or LC-LC MS/MS. This invention overcomes the limitations inherent in traditional techniques. The basic approach described can be employed for quantitative analysis of protein expression in complex samples (such as cells, tissues, and fraction etc.), the detection and quantitation of specific proteins in complex samples, and quantitative measurement of specific enzymatic activities in complexed samples.

Technical Description

1. Probe design:

The solid support part of the chemical probe may consist of any of the following materials or any combination of them: gel, glass beads, magnetic beads, polymers, silicon wafer, membrane, or resin.

The spacer between the solid phase part and the cleavable unit of the chemical probe may be included for convenience and improved yields in synthetic preparation of the chemical probe. The spacer may consist of a chain of 2 to 8 atoms, which can be C, O, N, B, Si, S, P, Se . . . , covalently bound to each other. In order to satisfy the valence requirements, the atoms may carry hydrogen atoms, halogens, or one of the following groups containing up to 25 atoms: alkyl, hydroxy, alkoxy, amino, alkylamino . . . The spacer may contain cyclic moieties with or without heteroatoms and with or without substituents.

The cleavable moiety provides means for selective detachment of the solid phase part of the chemical probe from the differential mass label attached to peptide. It is designed such that it can be cleaved by treating the probe with a chemical reagent or any kind of electromagnetic irradiation, photochemically, enzymatically, or thermally.

Differential mass labeling units differ in molecular mass, but do not differ in retention properties regarding the separation method used and in ionization and detection properties regarding the mass spectrometry methods used. These moieties differ either in their isotope composition (isotopic labels) or they differ structurally by a rather small fragment, which change does not alter the properties stated above (homologous labels).

The isotopic labels can be presented by general formulae:

Z^{A} and Z^{B}

Z^{A} and Z^{B}=R-Z¹-A¹-Z²-A² -Z³-A³-Z⁴-A⁴-

Z¹, Z², Z³, and Z⁴ independently of one another can be selected from O, OC (O), OC (S), OC (O) O, OC (O) NR, OC (S) NR, OSiRR¹, S, SC (O), SC (S), SS,S (O), S (O₂), NR, NRR¹⁺, C (I), C (O) O, C (S), C (S) O, C (O) S, C (O) NR, C (S) NR, SiRR¹, (Si (RR¹) O) n, SnRR¹, Sn (RR¹) O, BR (OR¹), BRR¹, B (OR)(OR¹), OBR (OR¹), OBRR¹, OB (OR)(OR¹) or Z¹-Z⁴ may be absent;

A¹, A², A³, and A⁴ independently of one another can be selected from (CRR¹)n, in which some single C—C bonds may be replaced with double or triple bonds, in which case some groups R and R¹ will be absent, o-arylene, m-arylene, p-arylene with up to 6 substituents, carbocyclic, bicyclic, or tricyclic fragments with up to 8 atoms in the cycle with or without heteroatoms (O, N, S) and with or without substituents, or A¹-A⁴ may be absent;

R, R¹ independently from other R and R¹ in Z¹-Z⁴ and independently from other R and R¹ in A¹-A⁴ is hydrogen, halogen, an alkyl, alkenyl, alkynyl, or aryl group;

n in Z¹-Z⁴ is independent of n in A¹-A⁴ and is a whole number that can have value from 0 to 21.

Z^{A} can have the same structure as Z^{B}, but they have different isotope composition. For instance, if Z^{A} contains x number of protons, Z^{B} may contain y number of deuterons in the place of protons, and, correspondingly, x-y number of protons remaining; and/or if Z^{A} contains x number of borons-10, Z^{B} may contain y number of borons-11 in the place of borons-10, and, correspondingly, x-y number of borons-10 remaining; and/or if Z^{A} contains x number of carbons-12, Z^{B} may contain y number of carbons-13 in the place of carbons-12, and, correspondingly, x-y number of carbons-12 remaining; and/or if Z^{A} contains x number of nitrogens-14, Z^{B} may contain y number of nitrogens-15 in the place of nitrogens-14, and, correspondingly, x-y number of nitrogens-14 remaining; and/or if Z^{A} contains x number of sulfurs-32, Z^{B} may contain y number of sulfurs-34 in the place of sulfurs-32, and, correspondingly, x-y number of sulfurs-32 remaining; and so on for all elements which may be present and have different stable isotopes; x and y are whole numbers between 1 and 21 such that x is greater than y.

An example of an isotopical label pairs/series: (CD₂)ₙ/(CH₂)ₙ, where n=0, 1, 2, . . . , 21; (delta mass=2n).

The homologous reagents can be presented by general formulae:

Z^{A} and Z^{B} where Z^{A} and Z^{B}=R-Z¹-A¹-Z²-A²-Z³-A³-Z⁴-A⁴-

Z¹, Z², Z³, and Z⁴ independently of one another can be selected from O, OC(O), OC(S), OC(O)O, OC(O)NR, OC(S)NR, OSiRR¹, S, SC(O), SC(S), SS, S(O), S(O₂), NR, NRR¹⁺, C(O), C(O)O, C(S), C(S)O, C(O)S, C(O)NR, C(S)NR, SiRR¹, (Si(RR¹)O)n, SNRR¹, Sn(RR¹)O, BR(OR¹), BRR¹, B(OR)(OR¹), OBR(OR¹), OBRR¹, OB(OR)(OR¹) or Z¹-Z⁴ may be absent;

A¹, A², A³, and A⁴ independently of one another can be selected from (CRR¹)n, in which some single C—C bonds may be replaced with double or triple bonds, in which case some groups R and R¹ will be absent, o-arylene, m-arylene, p-arylene with up to 6 substituents, carbocyclic, bicyclic, or tricyclic fragments with up to 8 atoms in the cycle with or without heteroatoms (O, N, S) and with or without substituents, or A¹-A⁴ may be absent;

R, R¹ independently from other R and R¹ in Z¹-Z⁴ and independently from other R and R¹ in A¹-A⁴ is hydrogen, halogen, an alkyl, alkenyl, alkynyl, or aryl group;

n in Z¹-Z⁴ is independent of n in A¹-A⁴ and is a whole number that can have value from 0 to 21.

Z^{A} can have a similar structure to that of Z^{B}, but Z^{A} has x extra —CH₂— fragment(s) in one or more A¹-A⁴ fragments, and/or Z^{A} has x extra —CF₂— fragment(s) in one or more A¹-A⁴ fragments; and/or if Z^{A} contains x number of protons, Z^{B} may contain y number of halogens in the place of protons, and, correspondingly, x-y number of protons remaining in one or more A¹-A⁴ fragments; and/or Z^{A} has x extra —O— fragment(s) in one or more A¹-A⁴ fragments; and/or Z^{A} has x extra —S— fragment(s) in one or more A¹-A⁴ fragments; and/or if Z^{A} contains x number of —O— fragment(s), Z^{B} may contain y number of —S— fragment(s) in the place of —O— fragment(s), and, correspondingly, x-y number of —O— fragment(s) remaining in one or more A¹-A⁴ fragments; and so on; x and y are whole numbers between 1 and 21 such that x is greater than y.

An examples of homologous label pairs/series: (CH₂)ₙ/(CH₂)ₙ₊ₘ, where n=0, 1, 2, . . . , 21; m=1, 2, . . . , 21 (delta mass=14m)

Bridging and Activating Reagents

In alternative aspects, commercially available cross linkers or custom designed cross-linked are used.

a. Reactive site 1: probe specific

b. Reactive site 2: amino acid specific

Methods for Peptide/Protein Separation and Detection

On line 2 dimensional capillary LC ESI MS/MS (MuDPIT) as described in the global differential profiling disclosure, or 1 D LC ESI MS/MS, MALDI MS.

Sequence Analysis and Quantification

Peptides are quantified by measuring in the MS mode the relative signal intensities for pairs or series of peptide ions of identical sequence that are tagged differentially, which therefore differ in mass by the mass differential encoded within the differential labeling reagents. Peptide sequence information is automatically generated by selecting peptide ions of a particular mass-to-charge (m/z) ratio for collision-induced dissociation (CID) in the mass spectrometer operating in the tandem MS mode. (Link et al, Electrophoresis 18:1314-34 (1997); Gygi et al. Nature Biotechnol 17:994-9) (1999); Gygi et al., cell Biol 19:1720-30 (1999)).

The resulting tandem mass spectra can be correlated to sequence databases to identify the protein from which the sequenced peptide originated. Currently commercial available softwares are Turbo SEQUEST by Thermofinigan, MassScot by Matrix Science, and Sonar MS/MS by Proteometrics. Special software development will be necessary for automated relative quantification.

Exemplary Approaches for Practicing the Invention:

1. Protein sample preparation, which may include protein denaturation, reduction, and proteolytic digestion

2. Treatment of the probe with a desired activating or bridging reagent

3. Treatment of the activated probe with a mixture of peptides

4. Wash off unbound peptides, which don't have the targeted amino acid

5. Combining modified differential labeled peptide mixture

6. Release peptides by cleaving the probe (steps 5 and 6 can be switched)

7. Removing solvent or desalting if necessary

8. Redisovling peptide in LC loading buffer

9. LC ESI MS and MS/MS analysis MALDI MS and MS/MS analysis

10. Database searching and data analysis

Metabolomics and Lipidomics

The invention also incorporates holistic monitoring approaches, metabolomics and lipidomics, including profiling metabolite pools, carbohydrates, lipids, glycoproteins, and glycolipids Various chromatographic methods and other qualitative and/or quantitative methods could be utilized to characterize lipid profiles. In the area of metabolomics, methods that compare concentrations of metabolites/small molecules, using a variety of chemical analysis tools, e.g. mass spec, NMR, other spectroscopic techniques, biosensors could be utilized.

For some specific method examples, see the following references: J. C. Lindon et al., Prog. NMR Spear., 29, 1 (1996)1-J. C. Lindon et al., Drug. Met. Rev., 29, 705 (1997); B. Vogler et al., J Nat. Prod., 61, 175 (1998); and J A. Wolfender et al., Curr. Org. Chem. 2, 575 (1998); J. K. Nicholson et al., Xenobiotica, 29, 1181(1999).

Screening Tools

FACS

In one aspect, fluorescence activated cell sorting (FACS) methods are used for selection/screening. In some instances a fluorescent molecule is made within a cell (e.g., green fluorescent protein). The cells producing the protein can simply be sorted by FACS. Gel microdrop technology allows screening of cells encapsulated in agarose microdrops (Weaver et al. Methods 2:234-247 (1991)). In this technique products secreted by the cell (such as antibodies or antigens) are immobilized with the cell that generated them. Sorting and collection of the drops containing the desired product thus also collects the cells that made the product, and provides a ready source for the cloning of the genes encoding the desired functions. Desired products can be detected by incubating the encapsulated cells with fluorescent antibodies (Powell et al. Bio/Technology 8:333-337 (1990)). FACS sorting can also be used by this technique to assay resistance to toxic compounds and antibiotics by selecting droplets that contain multiple cells (i.e., the product of continued division in the presence of a cytotoxic compound; Goguen et al. Nature 363:189-190 (1995)). This method can select for any enzyme that can change the fluorescence of a substrate that can be immobilized in the agarose droplet.

Reporter Molecule

In some aspects of the invention, screening can be accomplished by assaying reactivity with a reporter molecule reactive with a desired feature of, for example, a gene product. Thus, specific functionalities such as antigenic domains can be screened with antibodies specific for those determinants.

Cell-Cell Indicator

In other aspects of the invention, screening is done with a cell-cell indicator assay. In this assay format, separate library cells (Cell A, the cell being assayed) and reporter cells (Cell B, the assay cell) are used.

Only one component of the system, the library cells, is allowed to evolve. The screening is generally carried out in a two-dimensional immobilized format, such as on plates. The products of the metabolic pathways encoded by these genes (in this case, usually secondary metabolites such as antibiotics, polyketides, carotenoids, etc.) diffuse out of the library cell to the reporter cell. The product of the library cell may affect the reporter cell in one of a number of ways.

The assay system (indicator cell) can have a simple readout (e.g., green fluorescent protein, luciferase, beta-galactosidase) which is induced by the library cell product but which does not affect the library cell. In these examples the desired product can be detected by colorimetric changes in the reporter cells adjacent to the library cell.

Feedback Mechanism

In other aspects, indicator cells can in turn produce something that modifies the growth rate of the library cells via a feedback mechanism. Growth rate feedback can detect and accumulate very small differences. For example, if the library and reporter cells are competing for nutrients, library cells producing compounds to inhibit the growth of the reporter cells will have more available nutrients, and thus will have more opportunity for growth. This is a useful screen for antibiotics or a library of polyketide synthesis gene clusters where each of the library cells is expressing and exporting a different polyketide gene product.

Screening Secreted Molecules

Another variation of this theme is that the reporter cell for an antibiotic selection can itself secrete a toxin or antibiotic that inhibits growth of the library cell. Production by the library cell of an antibiotic that is able to suppress growth of the reporter cell will thus allow uninhibited growth of the library cell.

Conversely, if the library is being screened for production of a compound that stimulates the growth of the reporter cell (for example, in improving chemical syntheses, the library cell may supply nutrients such as amino acids to an auxotrophic reporter, or growth factors to a growth-factor-dependent reporter. The reporter cell in turn should produce a compound that stimulates the growth of the library cell. Interleukins, growth factors, and nutrients are possibilities. Further possibilities include competition based on ability to kill surrounding cells, positive feedback loops in which the desired product made by the evolved cell stimulates the indicator cell to produce a positive growth factor for cell A, thus indirectly selecting for increased product formation.

In some aspects of the invention it can be advantageous to use a different organism (or genetic background) for screening than the one that will be used in the final product. For example, markers can be added to DNA constructs used for recursive sequence recombination to make the microorganism dependent on the constructs during the improvement process, even though those markers may be undesirable in the final recombinant microorganism.

Likewise, in some aspects it is advantageous to use a different substrate for screening an evolved enzyme than the one that will be used in the final product. For example, Evnin et al. (Proc. Natl. Acad. Sci. U.S.A. 87:6659-6663 (1990)) selected trypsin variants with altered substrate specificity by requiring that variant trypsin generate an essential amino acid for an arginine auxotroph by cleaving arginine beta-naphthylamide. This is thus a selection for arginine-specific trypsin, with the growth rate of the host being proportional to that of the enzyme activity.

The pool of cells surviving screening and/or selection is enriched for recombinant genes conferring the desired phenotype (e.g. altered substrate specificity, altered biosynthetic ability, etc.). Further enrichment can be obtained, if desired, by performing a second round of screening and/or selection without generating additional diversity.

The recombinant gene or pool of such genes surviving one round of screening/selection forms one or more of the substrates for a second round of recombination. Again, recombination can be performed in vivo or in vitro by any of the recursive sequence recombination formats described above.

If recursive sequence recombination is performed in vitro, the recombinant gene or genes to form the substrate for recombination should be extracted from the cells in which screening/selection was performed. Optionally, a subsequence of such gene or genes can be excised for more targeted subsequent recombination. If the recombinant gene(s) are contained within episomes, their isolation presents no difficulties. If the recombinant genes are chromosomally integrated, they can be isolated by amplification primed from known sequences flanking the regions in which recombination has occurred. Alternatively, whole genomic DNA can be isolated, optionally amplified, and used as the substrate for recombination. Small samples of genomic DNA can be amplified by whole genome amplification with degenerate primers (Barrett et al. Nucleic Acids Research 23:3488-3492 (1995)). These primers result in a large amount of random 3′ ends, which can undergo homologous recombination when reintroduced into cells.

If the second round of recombination is to be performed in vivo, as is often the case, it can be performed in the cell surviving screening/selection, or the recombinant genes can be transferred to another cell type (e.g., a cell is type having a high frequency of mutation and/or recombination). In this situation, recombination can be effected by introducing additional DNA segment(s) into cells bearing the recombinant genes. In other methods, the cells can be induced to exchange genetic information with each other by, for example, electroporation. In some methods, the second round of recombination is performed by dividing a pool of cells surviving screening/selection in the first round into two subpopulations. DNA from one subpopulation is isolated and transfected into the other population, where the recombinant gene(s) from the two subpopulations recombine to form a further library of recombinant genes. In these methods, it is not necessary to isolate particular genes from the first subpopulation or to take steps to avoid random shearing of DNA during extraction. Rather, the whole genome of DNA sheared or otherwise cleaved into manageable sized fragments is transfected into the second subpopulation. This approach is particularly useful when several genes are being evolved simultaneously and/or the location and identity of such genes within chromosome are not known.

The second round of recombination is sometimes performed exclusively among the recombinant molecules surviving selection. However, in other aspects, additional substrates can be introduced. The additional substrates can be of the same form as the substrates used in the first round of recombination, i.e., additional natural or induced mutants of the gene or cluster of genes, forming the substrates for the first round. Alternatively, the additional substrate(s) in the second round of recombination can be exactly the same as the substrate(s) in the first round of replication.

After the second round of recombination, recombinant genes conferring the desired phenotype are again selected. The selection process proceeds essentially as before. If a suicide vector bearing a selective marker was used in the first round of selection, the same vector can be used again. Again, a cell or pool of cells surviving selection is selected. If a pool of cells, the cells can be subject to further enrichment.

Screening for Various Potential Applications

Novel Drugs: Identifying Targets

The invention relates to procedures that can be applied to identifying compounds that bind to and modulate the function of target components of a cell whose function is known or unknown, and cell components that are not amenable to other screening methods. The invention relates to generating and/or identifying a compound that binds to and modulates (inhibits or enhances) the function of a component of a cell, thereby producing a phenotypic effect in the cell. Such a screen may involve identifying a biomolecule that 1) binds to, in vitro, a component of a cell that has been isolated from other constituents of the cell and that 2) causes, in vivo, as seen in an assay upon intracellular expression of the biomolecule, a phenotypic effect in the cell which is the usual producer and host of the target cell component. In an assay demonstrating characteristic 2) above, intracellular production of the biomolecule can be in cells grown in culture or in cells introduced into an animal. Further methods within these procedures are those methods comprising an assay for a phenotypic effect in the cell upon intracellular production of the biomolecule, either in cells in culture or in cells that have been introduced into one or more animals, and an assay to identify one or more compounds that behave as competitors of the biomolecule in an assay of binding to the target cell component. The target cell component in this aspect and in other aspects not limited to pathogens can be one that is found in mammalian cells, especially cells of a type found to cause or contribute to disease or the symptoms of disease (e.g., cells of tumors or cells of other types of hyperproliferative disorders).

Process for Identifying One or More Compounds That Produce a Phenotypic Effect on a Cell

In one aspect, the invention provides a process for identifying one or more compounds that produce a phenotypic effect on a cell. The process is at the same time a method for target validation. The process is characterized by identifying a biomolecule which binds an isolated target cell component, constructing cells comprising the target cell component and further comprising a gene encoding the biomolecular binder which can be expressed to produce the biomolecular binder, testing the constructed cells for their ability to produce, upon expression of the gene encoding the biomolecular binder, a phenotypic effect in the cells (e.g., inhibition of growth), wherein the test of the constructed cells can be a test of the cells in culture or a test of the cells after introducing them into host animals, or both, and further, identifying, for a biomolecular binder that caused the phenotypic effect, one or more compounds that compete with the biomolecular binder for binding to the target cell component.

A test of the constructed cells after introducing them into host animals is especially well-suited to assessing whether a biomolecular binder can produce a particular phenotype by the expression (regulatable by the researcher) of a gene encoding the biomolecular binder. In this method, cells are constructed which have a gene encoding the biomolecular binder, and wherein the biomolecular binder can be produced by regulation of expression of the gene. The constructed cells are introduced into a set of animals. Expression of the gene encoding the biomolecular binder is regulated in one group of the animals (test animals) such that the biomolecular binder is produced. In another group of animals, the gene encoding the biomolecular binder is regulated such that the biomolecular binder is not produced (control animals). The cells in the two groups of animals are monitored for a phenotypic change (for example, a change in growth rate). If the phenotypic change is observed in cells in the test animals and not in the cells in the control animals, or to a lesser extent in the control animals, then the biomolecular binder has been proven to be effective in binding to its target cell component under in vivo conditions.

In one aspect of the invention is a method for determining whether a target cell component of a particular cell type (a “first cell”) is essential to producing a phenotypic effect on the first cell, the method having the steps:

isolating the target component of the first cell; identifying a biomolecular binder of the isolated target component of the first cell; constructing a second type of cells (“second cell”) comprising the target component and a regulable, exogenous gene encoding the biomolecular binder; and testing the second cell in culture for an altered phenotypic effect, upon production of the biomolecular binder in the second cell; whereby, if the second cell shows the altered phenotypic effect upon production of the biomolecular binder, then the target component of the first cell is essential to producing the phenotypic effect on the first cell. The target cell component in this aspect and in other aspects not limited to pathogens can be one that is found in mammalian cells, especially cells of a type found to cause or contribute to disease or the symptoms of disease (e.g., cells of tumors or cells of other types of hyperproliferative disorders).

Identifying a Biomolecular Inhibitor of Growth of Pathogen Cells

One aspect of the invention is a method for identifying a biomolecular inhibitor of growth of pathogen cells by using cell culture techniques, comprising contacting one or more types of biomolecules with isolated target cell component of the pathogen, applying a means of detecting bound complexes of biomolecules and target cell component, whereby, if the bound complexes are detected, one or more types of biomolecules have been identified as a biomolecular binder of the target cell component, constructing a pathogen strain having a regulatable gene encoding the biomolecular binder, regulating expression of the gene encoding the biomolecular binder to express the gene; and monitoring growth of the pathogen cells in culture relative to suitable control cells, whereby, if growth of the pathogen cells is decreased compared to growth of suitable control cells, then the biomolecule is a biomolecular inhibitor of growth of the pathogen cells.

Identifying Compounds That Inhibit Infection of a Mammal by a Pathogen

Another aspect of the invention is a method, employing an animal test, for identifying one or more compounds that inhibit infection of a mammal by a pathogen by binding to a target cell component, comprising constructing a pathogen comprising a regulatable gene encoding a biomolecule which binds to the target cell component, infecting test animals with the pathogen, regulating expression of the regulatable gene to produce the biomolecule, monitoring the test animals and suitable control animals for signs of infection, wherein observing fewer or less severe signs of infection in the test animals than in suitable control animals indicates that the biomolecule is a biomolecular inhibitor of infection, and identifying one or more compounds that compete with the biomolecular inhibitor of growth for binding to the target cell component (as by employing a competitive binding assay), then the compound inhibits infection of a mammal by a pathogen by binding to a target.

The competitive binding assay to identify binding analogs of biomolecular binders, which have been proven to bind to their targets in an intracellular test of binding, can be applied to any target for which a biomolecular binder has been identified, including targets whose function is unknown or targets for which other types of assays are not easily developed and performed. Therefore, the method of the invention offers the advantage of decreasing assay development time when using a gene product of known function as a target cell component and the advantage of bypassing the major hurdle of gene function identification when using a gene product of unknown function as a target cell component.

Other aspects of the invention are cells comprising a biomolecule and a target cell component, wherein the biomolecule is produced by expression of a regulable gene, and wherein the biomolecule modulates function of the target cell component, thereby causing a phenotypic change in the cells. Yet other aspects are cells comprising a biomolecule and a target cell component, wherein the biomolecule is a biomolecular binder of the target cell component, and is encoded by a regulatable gene. The cells can include mammalian cells or cells of a pathogen, for instance, and the phenotypic change can be a change in growth rate.

The pathogen can be a species of bacteria, yeast, fungus, or parasite, for example.

Intracellular Validation of a Biomolecule

The invention provides methods that result in the identification of compounds that cause a phenotypic effect on a cell. The general steps described herein to find a compound for drug development can be thought of as these: (1) identifying a biomolecule that can bind to an isolated target cell component in vitro, (2) confirming that the biomolecule, when produced in cells with the target cell component, can cause a desired phenotypic effect and (3) identifying, by an in vitro screening method, for example, compounds that compete with the biomolecule for binding to the target cell component. Central to these methods is general step (2) above, intracellular validation of a biomolecule comprising one or more steps that determine whether a biomolecule can cause a phenotypic effect on a cell, when the biomolecule is produced by the expression (which can be regulatable) of a gene in the cell. As used in general step (2), a biomolecule is a gene product (e.g., polypeptide, RNA, peptide or RNA oligonucleotide) of an exogenous gene—a gene which has been introduced in the course of construction of the cell.

Biomolecules that bind to and alter the function of a candidate target are identified by various in vitro methods. Upon production of the biomolecule within a cell either in vitro or within an animal model system, the biomolecule binds to a specific site on the target, alters its intracellular function, and hence produces a phenotypic change (e.g. cessation of growth, cell death). When the biomolecule is produced in engineered pathogen cells in an animal model of infection, cessation of growth or death of the engineered pathogen cells leads to the clearing of infection and animal survival, demonstrating the importance of the target in infection and thereby validating the target.

A further aspect of this invention provides for identifying a biomolecule that produces a phenotypic effect on a cell (wherein the cell can be, for instance, a pathogen cell or a mammalian cell) and (2) simultaneous intracellular target validation (see reference: Patents??).

Methods for Identifying Compounds That Inhibit the Growth of Cells Having a Target Cell Component

The invention includes methods for identifying compounds that inhibit the growth of cells having a target cell component. The target cell component can first be identified as essential to the growth of the cells in culture and/or under conditions in which it is desired that the growth of the cells be inhibited. These methods can be applied, for example, to various types of cells that undergo abnormal or undesirable proliferation, including cells of neoplasms (tumors or growths, either benign or malignant) which, as known in the art, can originate from a variety of different cell types. Such cells can be referred to, for example, as being from adenomas, carcinomas, lymphomas or leukemias. The method can also be applied to cells that proliferate abnormally in certain other diseases, such as arthritis, psoriasis or autoimmune diseases.

If intracellular expression of the biomolecular binder inhibits the function of a target essential for growth (presumably by binding to the target at a biologically relevant site) cells monitored in step (2) will exhibit a slow growth or no growth phenotype. Targets found to be essential for growth by these methods are validated starting points for drug discovery, and can be incorporated into assays to identify more stable compounds that bind to the same site on the target as the biomolecule. Where the cells are pathogen cells and the desired phenotypic change to be monitored is inhibition of growth, the invention provides a procedure to examine the activity of target (pathogen) cell components in an animal infection model.

Study as a Target Cell Component a Gene Product of a Particular Cell Type

In the course of this method, it may be decided to study as a target cell component a gene product of a particular cell type (e.g., a type of pathogenic bacteria), wherein the target cell component is already known as being encoded by a characterized gene, as a potential target for a modulator to be identified. In this case, the target cell component can be isolated directly from the cell type of interest, assuming suitable culture methods are available to grow a sufficient number of cells, using methods appropriate to the type of cell component to be isolated (e.g., protein purification methods such as differential precipitation, ion exchange chromatography, gel chromatography, affinity chromatography, HPLC.

Target Cell Component can be Produced Recombinantly

Alternatively, the target cell component can be produced recombinantly, that requires that the gene encoding the target cell component be isolated from the cell type of interest. This can be done by any number of methods, for example known methods such as PCR, using template DNA isolated from the pathogen or a DNA library produced from the pathogen DNA, and using primers based on known sequences or combinations of known and unknown sequences within or external to the chosen gene. See, for example, methods described in “The Polymerase Chain Reaction,” Chapter 15 of Current Protocols in Molecular Biology, (Ausubel, F. M. et al., eds), John Wiley & Sons, New York, 1998. Other methods include cloning a gene from a DNA library (e.g., a cDNA library from a eukaryotic pathogen) into a vector (e.g., plasmid, phage, phagemid, virus, etc.) and applying a means of selection or screening, to clones resulting from a transformation of vectors (including a population of vectors now having inserted genes) into appropriate host cells. The screening method can take advantage of properties given to the host cells by the expression of the inserted chosen gene (e.g., detection of the gene product by antibodies directed against it, detection of an enzymatic activity of the gene product), or can detect the presence of the gene itself (for instance, by methods employing nucleic acid hybridization). For methods of cloning genes in E. coli, which also may be applicable to cloning in other bacterial species, see, for example, “Escherichia coli, Plasmids and Bacteriophages,” Chapter I of Current Protocols in Molecular Biology, (Ausubel, F. M. et al., eds), John Wiley & Sons, New York, 1998. For methods applicable to cloning genes of eukaryotic origin, see Chapter 5 (“Construction of Recombinant DNA Libraries”), Chapter 9 (“Introduction of DNA Into Mammalian Cells”) and Chapter 6 (“Screening of Recombinant DNA Libraries”) of Current Protocols in Molecular Biology, (Ausubel, F. M. et al., eds), John Wiley & Sons, New York, 1998.

Target proteins can be expressed with E. coli or other prokaryotic gene expression systems, or in eukaryotic gene expression systems. Since many eukaryotic proteins carry unique modifications that are required for their activities, e.g. glycosylation and methylation, protein expression can in some cases be better carried out in eukaryotic systems, such as yeast, insect, or mammalian cells that can perform these modifications. Examples of these expression systems have been reviewed in the following literature: Methods in Enzymology, Volume 185, eds D. V. Goeddel, Academic Press, San Diego, 1990; Geisse et al, Protein Expression and Purification 8:271-282, 1996; Simonsen and McGrogan, Biologicals 22: 85-94; Jones and Morikawa, Current Opinions in Biotechnologies 7: 512-516, 1996; Possee, Current Opinions in Biotechnologies 8:569-572.

Where a gene encoding a chosen target cell component has not been isolated previously, but is thought to exist because homologs of the gene product are known in other species, the gene can be identified and cloned by a method such as that used in Shiba et al., U.S. Pat. No. 5,759,833, Shiba et al., U.S. Pat. No. 5,629,188, Martinis et al., U.S. Pat. No. 5,656,470 and Sassanfar et al., U.S. Pat. No. 5,756,327.

Method Should be Used With Target Cell Components Which Have Not Been Previously Isolated or Characterized and Whose Functions are Unknown

It is an advantage of the target validation method that it can be used with target cell components which have not been previously isolated or characterized and whose functions are unknown. In this case, a segment of DNA containing an open reading frame (ORF; a cDNA can also be used, as appropriate to a eukaryotic cell) which has been isolated from a cell of a type that is to be an object of drug action (e.g., tumor cell, pathogen cell) can be cloned into a vector, and the target gene product of the ORF can be produced in host cells harboring the vector. The gene product can be purified and further studied in a manner similar to that of a gene product that has been previously isolated and characterized.

In some cases, the open reading frame (in some cases, cDNA) can be isolated from a source of DNA of the cells of interest (genomic DNA or a library, as appropriate), and inserted into a fusion protein or fusion polypeptide construct. This construct can be a vector comprising a nucleic acid sequence which provides a control region (e.g., promoter, ribosome binding site) and a region which encodes a peptide or polypeptide portion of the fusion polypeptide wherein the polypeptide encoded by the fusion vector endows the fusion polypeptide with one or more properties that allow for the purification of the fusion polypeptide. For example, the vector can be one from the pGEX series of plasmids (Pharmacia) designed to produce fusions with glutathione S-transferase.

Host Cells

The isolated DNA having an open reading frame, whether encoding a known or an as yet unidentified gene product, when inserted into an expression construct, can be expressed to produce the target cell component in host cells. Host cells can be, for example, Gram-negative or Gram-positive bacterial cells such as Escherichia coli or Bacillus subtilis, respectively, or yeast cells such as Saccharomyces cerevisiae, Schizosaccharomyces pombe or Pichia pastoris. In one aspect, the target cell component can be used in target validation studies be produced in a host that is genetically related to the pathogen from which the gene encoding it was isolated. For example, for a Gram-negative bacterial pathogen, an E. coli host is preferred over a Pichia pastoris host. The target cell component so produced can then be isolated from the host cells. Many protein purification methods are known that separate proteins on the basis of, for instance, size, charge, or affinity for a binding partner (e.g., for an enzyme, a binding partner can be a substrate or substrate analog), and these methods can be combined in a sequence of steps by persons of skill in the art to produce an effective purification scheme. For methods to manipulate RNA, see, for example, Chapter 4 in Current Protocols in Molecular Biology (Ausubel, F. M. et al., eds), John Wiley & Sons, New York, 1998.

An isolated cell component or a fusion protein comprising the cell component can be used in a test to identify one or more biomolecular binders of the isolated product (general step (1)). A biomolecular binder of a target cell component can be identified by in vitro assays that test for the formation of complexes of target and biomolecular binder no covalently, bound to each other. For example, the isolated target can be contacted with one or more types of biomolecules under conditions conducive to binding, the unbound biomolecules can be removed from the targets, and a means of detecting bound complexes of biomolecules and targets can be applied. The detection of the bound complexes can be facilitated by having either the potential biomolecular binders or the target labeled or tagged with an adduct that allows detection or separation (e. g., radioactive isotope or fluorescent label; streptavidin, avidin or biotin affinity label).

Alternatively, both the potential biomolecular binders and the target can be differentially labeled. For examples of such methods see, e.g., WO 98/19162.

Biomolecules to be Tested and Means for Detection

The biomolecules to be tested for binding to a target can be from a library of candidate biomolecular binders, (e.g., a peptide or oligonucleotide library). For example, a peptide library can be displayed on the coat protein of a phage (see, for examples of the use of genetic packages such as phage display libraries, Koivunen, E. et al., J Biol. Chem. 268:20205-20210 (1993)). The biomolecules can be detected by means of a chemical tag or label attached to or integrated into the biomolecules before they are screened for binding properties. For example, the label can be a radioisotope, a biotin tag, or a fluorescent label. Those molecules that are found to bind to the target molecule can be called biomolecular binders.

Fusion Proteins

An isolated target cell component, an antigenically similar portion thereof, or a suitable fusion protein comprising all of or a portion of or the entire target can be used in a method to select and identify biomolecules which bind specifically to the target. Where the target cell component comprises a protein, fusion proteins comprising all of, or a portion of, the target linked to a second moiety not occurring in the target as found in nature, can be prepared for use in another aspect of the method. Suitable fusion proteins for this purpose include those in which the second moiety comprises an affinity ligand (e.g., an enzyme, antigen, epitope). The fusion proteins can be produced by the insertion of a gene encoding a target or a suitable portion of such gene into a suitable expression vector, which encodes an affinity ligand (e.g., pGEX-4T-2 and pET-15b, encoding glutathione S-transferase and His-Tag affinity ligands, respectively). The expression vector can be introduced into a suitable host cell for expression. Host cells are lysed and the lysate, containing fusion protein, can be bound to a suitable affinity matrix by contacting the lysate with an affinity matrix under conditions sufficient for binding of the affinity ligand portion of the fusion protein to the affinity matrix.

Fusion Protein can be Immobilized

In one aspect, the fusion protein can be immobilized on a suitable affinity matrix under conditions sufficient to bind the affinity ligand portion of the fusion protein to the matrix, and is contacted with one or more candidate biomolecules (e.g., a mixture of peptides) to be tested as biomolecular binders, under conditions suitable for binding of the biomolecules to the target portion of the bound fusion protein. Next, the affinity matrix with bound fusion protein can be washed with a suitable wash buffer to remove unbound biomolecules and non-specifically bound biomolecules. Biomolecules which remain bound can be released by contacting the affinity matrix with fusion protein bound thereto with a suitable elution buffer. Wash buffer can be formulated to permit binding of the fusion protein to the affinity matrix, without significantly disrupting binding of specifically bound biomolecules. In this aspect, elution buffer can be formulated to permit retention of the fusion protein by the affinity matrix, but can be formulated to interfere with binding of the test biomolecule(s) to the target portion of the fusion protein. For example, a change in the ionic strength or pH of the elution buffer can lead to release of biomolecules, or the elution buffer can comprise a release component or components designed to disrupt binding of biomolecules to the target portion of the fusion protein.

Immobilization can be performed prior to, simultaneous with, or after contacting, the fusion protein with biomolecule, as appropriate. Various permutations of the method are possible, depending upon factors such as the biomolecules tested, the affinity matrix-ligand pair selected, and elution buffer formulation. For example, after the wash step, fusion protein with biomolecules bound thereto can be eluted from the affinity matrix with a suitable elution buffer (a matrix elution buffer, such as glutathione for a GST fusion). Where the fusion protein comprises a cleavable linker, such as a thrombin cleavage site, cleavage from the affinity ligand can release a portion of the fusion with the biomolecules bound thereto. Bound biomolecule can then be released from the fusion protein or its cleavage product by an appropriate method, such as extraction.

Various Methods to Identify Biomolecular Binders

In one aspect, one or more candidate biomolecular binders can be tested simultaneously. Where a mixture of biomolecules is tested, the biomolecules selected by the foregoing processes can be separated (as appropriate) and identified by suitable methods (e.g., PCR, sequencing, chromatography). Large libraries of biomolecules (e.g., peptides, RNA oligonucleotides) produced by combinatorial chemical synthesis or other methods can be tested (see e. a., Ohlmeyer, M. H. J. et al., Proc. Natl. Acad. Sci. USA 90:10922-10926 (1993) and DeWitt, S. H. et al., Proc. Natl. Acad. Sci. USA 90:6909-6913 (1993), relating to tagged compounds; see also Rutter, W. J. et al. U.S. Pat. No. 5,010,175; Huebner, V. D. et al., U.S. Pat. No. 5,182,366; and Geysen, H. M., U.S. Pat. No. 4,833,092). Random sequence RNA libraries (see Ellington, A. D. et al., Nature 346:818-822 (1990); Bock, L. C. et al., Nature 355:584-566 (1992); and Szostak, J. W., Trends in Biochem. Sci. 17:89-93 (March, 1992)) can also be screened according to the present method to select RNA molecules which bind to a target. Where biomolecules selected from a combinatorial library by the present method carry unique tags, identification of individual biomolecules by chromatographic methods is possible. Where biomolecules do not carry tags, chromatographic separation, followed by mass spectrometry to ascertain structure, can be used to identify individual biomolecules selected by the method, for example.

Other methods to identify biomolecular binders of a target cell component can be used. For example, the two-hybrid system or interaction trap is an in vivo system that can be used to identify polypeptides, peptides or proteins (candidate biomolecular binders) that bind to a target protein. In this system, both candidate biomolecular binders and target cell component proteins are produced as fusion proteins. The two-hybrid system and variations on it have been described (U.S. Pat. No. 5,283,173 and U.S. Pat. No. 5,468,614; Golemis, E. A. et al., pages 20.1.1-20.1.35 In Current Protocols in Molecular Biology, F. M. Ausubel et al., eds., John Wiley and Sons, containing supplements up through Supplement 40, 1997; two-hybrid systems available from Clontech, Palo Alto, Calif.).

Once one or more biomolecular binders of a cell component have been identified, further steps can be combined with those taken to identify the biomolecular binder, to identify those biomolecular binders that produce a phenotypic effect on a cell (where “a cell” can mean cells of a cell strain or cell line).

Thus, a method for identifying a biomolecule that produces a phenotypic effect on a first cell can comprise the steps of identifying a biomolecular binder of an isolated target cell component of the first cell, constructing a second cell comprising the target cell component and a regulable exogenous gene encoding the biomolecular binder, and testing the second cell for the phenotypic effect, upon production of the biomolecular binder in the second cell, where the second cell can be maintained in culture or introduced into an experimental animal. If the second cell shows the phenotypic effect upon intracellular production of the biomolecular binder, then a biomolecule that produces a phenotypic effect on the first cell has been identified. Testing the second cell is general step (2) of the invention, as the three general steps were outlined above.

Host Cells: Engineered to Control Expression

Host cells (also, “second cells” in the terminology used above) of the cell type (e.g., species of pathogenic bacteria) the target was isolated from (or the gene encoding the target was originally isolated from, if the target is produced by recombinant methods), can be engineered to harbor a gene that can regulatably express the biomolecular binder (e.g., under an inducible or repressible promoter). The ability to regulate the expression of the biomolecular binder is desirable because constitutive expression of the biomolecular binder could be lethal to the cell.

Therefore, inducible or regulated expression gives the researcher the ability to control if and when the biomolecular binder is expressed. The gene expressing the biomolecular binder can be present in one or more copies, either on an extra chromosomal structure, such as on a single or multicopy plasmid, or integrated into the host cell genome. Plasmids that provide an inducible gene expression system in pathogenic organisms can be used. For example, plasmids allowing tetracycline-inducible expression of a gene in Staphylococcus aureus have been developed.

Genes for Expression

For intracellular expression of a biomolecule to be tested for its phenotypic effect in a eukaryotic cell (e.g., mammalian cell), the genes for expression can be carried on plasmid-based or virus-based vectors, or on a linear piece of DNA or RNA. For examples of expression vectors, see Hosfield and Lu, Biotechniques: 306-309, 1998; Stephens and Cockett, Nucleic Acid Research 17:7110, 1989; Wohlgemuth et al, Gene Therapy, 3:503-512, 1996; Ramirez-Solis et al, Gene 87:291-294, 1990, Dirks et al, Gene 149:387-388, 1994; Chenaalvala et al. Current Opinion in Biotechnologies 2:718-722, 1991; Methods in Enzymology, Volume 185, (D. V. Goeddel, ed.) Academic Press, San Diego, 1990. The genetic material can be introduced into cells using a variety of techniques, including whole cell or protoplast transformation, electroporation, calcium phosphate-DNA precipitation or DEAE-Dextran transfection, liposome mediated DNA or RNA transfer, or transduction with recombinant viral or retroviral vectors. Expression of the gene can be constitutive (e.g., ADHI promoter for expression in S. cerevisiae (Bennetzen, J. L. and Hall, B. D., J Biol. Chem 257:3026-3031 (1982)), or CMV immediate early promoter and RSV LTR for mammalian expression) or inducible, as the inducible GAL I promoter in yeast (Davis, L. I. and Fink, G. R., Cell 61:965-978 (1990)). A variety of inducible systems can be utilized, for example, E. coli Lac repressor/operator system and Tn10 Tet repressor/operator systems have been engineered to govern regulated expression in organisms from bacterial to mammalian cells. Regulated gene expression can also be achieved by activation. For example, gene expression governed by HIV LTR can be activated by HIV or SIV Tat proteins in human cells; GAL4 promoter can be activated by galactose in a nonglucose-containing medium. The location of the biomolecule binder genes can be extra chromosomal or chromosomally integrated. The chromosome integration can be mediated through homologous or nonhomologous recombinations.

For proper localization in the cells, it maybe desirable to tag the biomolecule binders with certain peptide signal sequences (for example, nuclear localization signal (NLS) sequences, mitochondria localization sequences). Secretion sequences have been well documented in the art.

Fused Biomolecular Binders

For presentation of the biomolecular binders in the intracellular system, they can be fused N-terminally, C-terminally, or internally in a carrier protein (if the biomolecular binder is a peptide), and can be fused (5′, 3′ or internally) in a carrier RNA or DNA molecule (if the biomolecular binder is a nucleic acid). The biomolecular binder can be presented with a protein or nucleic acid structural scaffold. Certain linkages (e.g., a 4-glycine linker for a peptide or a stretch of A's for an RNA can be inserted between the biomolecular binder and the carrier proteins or nucleic acids.

In such engineered cells, the effect of this biomolecular binder on the phenotype of the cells can be tested, as a manifestation of the binding (implying binding to a functionally relevant site, thus, an activator, or more likely, an inhibitory) effect of the biomolecular binder on the target used in an in vitro binding assay as described above. An intracellular test can not only determine which biomolecular binders have a phenotypic effect on the cells, but at the same time can assess whether the target in the cells is essential for maintaining the normal phenotype of the cells. For example, a culture of the engineered cells expressing a biomolecular binder can be divided into two aliquots. The first aliquot (“test” cells) can be treated in a suitable manner to regulate (e.g., induce or release repression of, as appropriate) the gene encoding the biomolecular binder, such that the biomolecular binder is produced in the cells. The second aliquot (“control” cells) can be left untreated so that the biomolecular binder is not produced in the cells. In a variation of this method of testing the effect of a biomolecular binder on the phenotype of the cells, a different strain of cells, not having a gene that can express the biomolecular binder, can be used as control cells. The phenotype of the cells in each culture (“test” and “control” cells grown under the same conditions, other than the expression of the biomolecular binder), can then be monitored by a suitable means (e.g., enzymatic activity, monitoring, a product of a biosynthetic pathway, antibody to test for presence of cell surface antigen, etc.). Where the change in phenotype is a change in growth rate, the growth of the cells in each culture (“test” and “control” cells grown under the same conditions, other than the expression of the biomolecular binder), can be monitored by a suitable means (e.g., turbidity of liquid cultures, cell count, etc). If the extent of growth, or rate of growth of the test cells is less than the extent of growth or rate of growth of the control cells, then the biomolecular binder can be concluded to be an inhibitor of the growth of the cells, or a biomolecular inhibitor.

If the phenotype of the test cells is altered relative to that of the control cells, then the biomolecular binder can be concluded to be one that causes a phenotypic effect. In an optional additional test, isolated target cell component having a known function (e.g., an enzyme activity) can be tested for modulation of this known function in the presence of biomolecular binder under conditions conducive to binding of the biomolecular binder to the target cell component. Positive results in these tests should encourage the investigator to continue in the drug discovery process with efforts to find a more stable compound (than a peptide, polypeptide or RNA biomolecule) that mimics the binding properties of the biomolecular binder on the tested target cell component.

Engineering Strain of Cells

A further test can, again, employ an engineered strain of cells that comprise both the target cell component and one or more genes encoding a biomolecule tested to be a biomolecular binder of the target cell component. The cells of the cell strain can be tested in animals to see if regulable expression of the biomolecular binder in the engineered cells produces an observable or testable change in phenotype of the cells. Both the “in culture” test for the effect of intracellular expression of the biomolecular binder and the “in animal” test (described below) for the effect of intracellular expression of the biomolecular binder can be applied not only towards drug discovery in the categories of antimicrobials and anticancer agents, but also towards the discovery of therapeutic agents to treat inflammatory diseases, cardiovascular diseases, diseases associated with metabolic pathways, and diseases associated with the central nervous system, for example.

Where the engineered strain of cells is a strain of pathogen cells or tumor cells, the object of the test is to see whether production of the biomolecular binder in the engineered strain inhibits growth of these cells after their introduction into an animal by the engineered pathogen. Such a test can not only determine which biomolecular binders are inhibitors of growth of the cells, but at the same time can assess whether the target in the cells is essential for maintaining growth of the cells (infection, for a pathogenic organism) in a host mammal. Suitable animals for such an experiment are, for example, mammals such as mice, rats, rabbits, guinea pigs, dogs, pigs, and the like. Small mammals can be used for reasons of convenience.

The engineered cells are introduced into one or more animals (“test” animals) and into one or more animals in a separate group (“control” animals) by a route appropriate to cause symptoms of systemic or local growth of the engineered cells. The route of introduction may be, for example, by oral feeding, by inhalation, by subdermal, intramuscular, intravenous, or intraperitoneal injection as appropriate to the desired result. After the cell strain has been introduced into the test and control animals, expression of the gene encoding the biomolecular binder is regulated to allow production of the biomolecular binder in the engineered pathogen cells. This can be achieved, for instance, by administering to the test animals a treatment appropriate to the regulation system built into the cells, to cause the gene encoding the biomolecular binder to be expressed. The same treatment is not administered to the control animals, but the conditions under which they are maintained are otherwise identical to those of the test animals. The treatment to express the gene encoding the biomolecular binder can be the administration of an inducer substance (where expression of the biomolecular binder or gene is under the control of an inducible promoter) or the functional removal of a repressor substance (where expression of the biomolecular binder gene is under the control of a repressible promoter).

After such treatment, the test and control animals can be monitored for a phenotypic effect in the introduced cells. Where the introduced cells are constructed pathogen cells, the animals can be monitored for signs of infection (as the simplest endpoint, death of the animal, but also e.g., lethargy, lack of grooming behavior, hunched posture, not eating, diarrhea or other discharges; bacterial titer in samples of blood or other cultured fluids or tissues). In the case of testing engineered tumor cells, the test and control animals can be monitored for the development of tumors or for other indicators of the proliferation of the introduced engineered cells. If the test animals are observed to exhibit less growth of the introduced cells than the control animals, then the biomolecule can be also called a biomolecular inhibitor of growth, or biomolecular inhibitor of infection, as appropriate, as it can be concluded that the expression in vivo of the biomolecular inhibitor is the cause of the relative reduction in growth of the introduced cells in the test animals.

In Vitro Assays

In alternative aspects, further steps of the procedure involve in vitro assays to identify one or more compounds that have binding and activating or inhibitory properties that are similar to those of the biomolecules which have been found to have a phenotypic effect, such as inhibition of growth. That is, compounds that compete for binding to a target cell component with the biomolecule would then be structural analogs of the biomolecules. Assays to identify such compounds can take advantage of known methods to identify competing molecules in a binding assay. These steps comprise general step (3) of the method.

In one method to identify such compounds, a biomolecular inhibitor (or activator) can be contacted with the isolated target-cell component to allow binding, one or more compounds can be added to the milieu comprising the biomolecular inhibitor and the cell component under conditions that allow interaction and binding between the cell component and the biomolecular inhibitor, and any biomolecular inhibitor that is released from the cell component can be detected.

Fluorescence

One suitable system that allows the detection of released biomolecular inhibitor (or activator) is one in which fluorescence polarization of molecules in the milieu can be measured. The biomolecular inhibitor can have bound to it a fluorescent tag or label such as fluorescein or fluorescein attached to a linker. Assays for inhibition of the binding of the biomolecular inhibitor to the cell component can be done in microtiter plates to conveniently test a set of compounds at the same time. In such assays, a majority of the fluorescently labeled biomolecular inhibitor must bind to the protein in the absence of competitor compound to allow for the detection of small changes in the bound versus free probe population when a compound which is a competitor with a biomolecular inhibitor is added (B. A. Lynch, et al., Analytical Biochemistry 247:77-82 (1997)). If a compound competes with the biomolecular inhibitor for a binding site on the target cell component, then fluorescently labeled biomolecular inhibitor is released from the target cell component, lowering the polarization measured in the milieu.

Radioactive Isotope

In a further method for identifying one or more compounds that compete with a biomolecular inhibitor (or activator) for a binding site on a target cell component, the target cell component can be attached to a solid support, contacted with one or more compounds, and contacted with the biomolecular inhibitor. One or more washing steps can be employed to remove biomolecular inhibitor and compound not bound to the cell component. Either the biomolecular inhibitor bound to the target cell component or the compound bound to the target cell component can be measured. Detection of biomolecular inhibitor or compound bound to the cell compound can be facilitated by the use of a label on either molecule type, wherein the label can be, for instance, a radioactive isotope either incorporated into the molecule itself or attached as an adduct, streptavidin or biotin, a fluorescent label or a substrate for an enzyme that can produce from the substrate a colored or fluorescent product. An appropriate means of detection of the labeled biomolecular inhibitor or compound moiety of the biomolecular inhibitor-cell component complex or the compound-cell component complex can be applied. For example, a scintillation counter can be used to measure radioactivity. Radio labeled streptavidin or biotin can be allowed to bind to biotin or streptavidin, respectively, and the resulting complexes detected in a scintillation counter. Alkaline phosphatase conjugated to streptavidin can be added to a biotin-labeled biomolecular inhibitor or compound. Detection and quantitation of a biotin-labeled complex can then be by addition of pNPP substrate of alkaline phosphatase and detection by spectrophotometry, of a product which absorbs UV light at a wavelength of 405 nm. A fluorescent label can also be used, in which case detection of fluorescent complexes can be by a fluorometer. Models are available that can read multiple samples, as in a microtiter plate.

For example, in one type of assay, the method for identifying compounds comprises attaching the target cell component to a solid support, contacting the biomolecular inhibitor with the target cell component under conditions suitable for binding of the biomolecular inhibitor to the cell component, removing unbound biomolecular inhibitor from the solid support, contacting one or more compounds (e.g., a mixture of compounds) with the cell component under conditions suitable for binding of the biomolecular inhibitor to the cell component, and testing for unbound biomolecular inhibitor released from the cell component, whereby if unbound biomolecular inhibitor is detected, one or more compounds that displace or compete with the biomolecular inhibitor for a particular site on the target cell component have been identified.

Other methods for identifying compounds that are competitive binders with the biomolecule for a target can employ adaptations of fluorescence polarization methods. See, for instance, Anal. Biochem. 253(2):210-218 (1997), Anal. Biochem. 249(1):29-36 (1997), BioTechniques 17(3):585-589 (1994) and Nature 373:254-256 (1995). Those compounds that bind competitively to the target cell component can be considered to be drug candidates. Further appropriate testing can confirm that those compounds which bind competitively with biomolecular inhibitors (or activators) possess the same activity as seen in an intracellular test of the effect of the biomolecular inhibitor or activator upon the phenotype of cells. Derivatives of these compounds having modifications to confer improved solubility, stability, etc., can also be tested for a desired phenotypic effect.

Combining Steps

Combining steps for testing the phenotypic effects of a biomolecule, as can be produced in an intracellular test, with steps for identifying compounds that compete with the biomolecule for sites on a target cell component, yields a method for identifying a compound which is a functional analog of a biomolecule which produces a phenotypic effect on a cell. These steps can be to test, for the phenotypic effect, either in culture or in an animal model, or in both, a cell which produces a biomolecule by regulatable expression of an exogenous gene in the cell, and to identify, if the biomolecule caused the phenotypic effect, one or more compounds that compete with the biomolecule for binding to a target cell component. If a compound is found to compete with the biomolecule for binding to the target cell component, then the compound is a functional analog of a biomolecule which produces a phenotypic effect on the cell. Such a functional analog can cause qualitatively a similar effect on the cell, but to a similar degree, lesser degree or greater degree than the biomolecule.

Method for Determining Whether a Target Component of a Cell is Essential to Producing a Phenotypic Effect on the Cell

A further aspect of the invention combining general steps (1) and (2) is a method for determining whether a target component of a cell is essential to producing a phenotypic effect on the cell, comprising isolating the target component from the cell, identifying a biomolecular binder of the isolated target component of the cell, constructing a second cell comprising the target component and a regulable, exogenous gene encoding the biomolecular binder, and testing the second cell in culture for an altered phenotypic effect, upon production of the biomolecular binder in the second cell, whereby, if the second cell shows the altered phenotypic effect upon production of the bimolecular binder, then the target component of the first cell is essential to producing the phenotypic effect on the first cell.

Inhibit the Proliferation of the Cells

The methods described herein are well suited to the identification of compounds that can inhibit the proliferation of the cells of infectious agents such as bacteria, fungi and the like. In addition, a procedure such as the one outlined below can be used in the identification of compounds to inhibit the proliferation of cancer cells. The two procedures described below further illustrate the use of the methods described herein and would provide proof of principle of these methods with a known target for anticancer therapy.

Mammalian dihydrofolate reductase (DHFR) is a proven target for anticancer therapy. Methotrexate (MTX) is one of many existing drugs that inhibit DHFR. It is widely used for anticancer chemotherapy.

NIH 3T3 is a mouse fibroblast cell line that is able to develop spontaneous transformed cells when cultured in low concentration (2%) of calf serum in molecular, cellular and developmental biology medium 402 (MCDB) (M. Chow and H. Rubin, Proc. Natl. Acad. Sci. USA 95(8):4550-4555 (1998)). The transformed cells, which can be selectively inhibited by MTX (Chow and Rubin), are isolated.

Both the normal and transformed NIH3T3 cells are transfected with pTet-On plasmid (Clontech; Palo Alto, Calif.). Stable cell lines that express high levels of reverse tetracycline-control led activator (rtTA) are isolated and characterized for their normal or transformed phenotype (Chow and Rubin).

The DHFR gene (Genbank Accession # L26316) from the NIH 3T3 cell line is amplified by reverse transcription-PCR (RT-PCR) using poly A′ RNA isolated from NIH 3T3 cells (Sambrook, J. et al., Molecular Cloning: A Laboratory Manual, 2nd edition, Cold Spring Harbor Laboratory Press, 1989). Active DHFR is expressed using the BacPAK Baculovirus Expression System (Clontech) or other appropriate systems. The expressed DHFR is purified and biotinylated and subjected to peptide binder identification as exemplified for bacterial proteins. The identified peptides are biochemically characterized for in vitro inhibition of DHFR activity. Peptides that inhibit DHFR are identified. A nucleic acid encoding each peptide can be cloned into a vector such as pGEX-4T2 (Pharmacia) to yield a vector which encodes a fusion polypeptide having the peptide fused to the N-terminus of GST. This can also be done by PCR amplification as exemplified herein for the peptide Pro-3. The fusion genes are cloned into plasmid pTRE (Clontech) for regulated expression. The constructed plasmid or the vector is co-transfected with pTK-Hyg into the stable NIH 3T3 cell line that expresses rtTA. The resulting cell lines, termed 3T3N-VITA (normal 3T3 cells that express rtTA and the DHFR inhibitory peptides), 3T3T-VITA (transformed 3T3 cells that express rtTA and the DHFR inhibitory peptides), or 3T3T-VITA control (transformed 3T3 cells that express rtTA and GST), are characterized for their normal or transformed phenotype (loss of contact inhibition, change in morphology, immortalization, etc. ). 10²-10¹ of 3T3T-VITA or 3T3T-VITA control cells are mixed with 10⁵ 3T3N-VITA and are grown in MCD 402 medium with 10% calf serum at 37° C for three days. Tetracycline is added to the medium to a final concentration of 0 to 1 ug/ml. In a control, 200 mM of MTX is added. The cultures are incubated for an additional eight days, and the number of foci formed are counted as described by M. Chow and H. Rubin, Proc. Natl. Acad Sci. USA 95(8):4550-4555 (1998). Peptides that specifically inhibit foci formation of 3T3 transformed cells are identified.

A murine model of fibroblastoma (Kogerman, P. et al., Oncogene (12):1407-1416 (1997)) is used for evaluating the DHFR/peptide combination for identification of compounds for cancer therapy. Various amounts of 3T3T-VITA or 3T3T-VITA control cells (10³, 10⁴, 10⁵, 10⁶ cells) are injected subcutaneously into 5 groups (10 in each group) of athymic nude mice (4-6 weeks old, 18-22 g) to determine the minimal dose needed for development of fibroblastomas in all of the tested animals. Upon determination of the minimal tumorigenic dose, 6 groups of athymic nude mice (10 each) are injected subcutaneously (s.c.) with the minimal tumorigenic dose for 3T3T-VITA or 3T3T-VITA control cells to develop fibroblastoma. One week after injection, group I mice start receiving MTX s.c. at 2 mg/kg/day as positive control, group 2 to 5 start receiving 1, 2, 5, or 10 mg/kg/day of tetracycline, group 6 start receiving saline (vehicle) as control. Five weeks after the introduction of cells, all of the mice are sacrificed and tumors are removed from them. Tumor mass is measured and compared among the groups.

An effective peptide identified by these in vivo experiments can be used for screening libraries of compounds to identify those compounds that competitively bind to DHFR. One mechanism of tumorigenesis is overexpression of proto-oncogenes such as Ha-ras (Reviewed by Suarez, H. G., Anticancer Research 9(5):1331-1343 (1989)).

Compounds that inhibit the activities of the products of such proto-oncogenes can be used for cancer chemotherapy. What follows is a further illustration of the methods described herein, as applied to mammalian cells.

Transgenic mice that overexpress human Ha-ras have been produced. Such transgenic mice develop salivary and/or mammary adenocarcinomas (Nielsen, L. L. et al, In Vivo 8(5):1331-1343 (1994)). Secondary transgenic mice that express rtTA can be generated using the pTet-On plasmid from Clontech.

Human Ha-ras open reading frame cDNA (Genbank Accession #G00277) is amplified by RT-PCR using polyA-RNA isolated from human mammary gland or other tissues. Active Ha-ras is expressed using the BacPAK Baculovirus Expression System (Clontech) or other appropriate systems. The expressed Ha-ras is purified and biotinylated and subjected to peptide binder identification as exemplified herein for bacterial proteins as target cell components. The identified peptides are biochemically characterized for in vitro inhibition of Ha-ras GTPase activity.

Peptides that inhibit Ha-ras are cloned into plasmid pTPE (Clontech) for regulated expression as an N-terminal fusion of GST. Such constructs are used to generate tertiary transgenic mice using the secondary transgenic mice. Transgenic mice that are able to overexpress peptide genes are identified by Northern and Western analysis. Control mice that express GST are also identified.

Various doses of tetracycline are administered to the tertiary transgenic mice by s.c. or I.P. injection before or after tumor onset. Prevention or regression of tumors resulting from expression of the peptide genes are analyzed as described above for murine fibroblastoma.

Peptides found to be effective in in vivo experiments will be used to screen compounds that inhibit human Ha-ras activity for cancer therapy.

Disease Targets

The method of the invention can be applied more generally to mammalian diseases caused by: (1) loss or gain of protein function, (2) over-expression or loss of regulation of protein activity. In each case the starting point is the identification of a putative protein target or metabolic pathway involved in the disease. The protocol can sometimes vary with the disease indication, depending on the availability of cell culture and animal model systems to study the disease. In all cases the process can deliver a validated target and assay combination to support the initiation of drug discovery.

Appropriate disease indications include, but are not limited to, Alzheimer's, arthritis, cancer, cardiovascular diseases, central nervous system disorders, diabetes, depression, hypertension, inflammation, obesity and pain.

Appropriate protein targets putatively linked to disease indications include, but are not limited to (1) the leptin protein, putatively linked to obesity and diabetes; (2) a mitogen-activated protein kinase putatively linked to arthritis, osteoporosis and atherosclerosis; (3) the interleukin-1 beta converting protein putatively linked to arthritis, asthma and inflammation; (4) the caspase proteins putatively linked to neurodegenerative diseases such as Alzheimer's, Parkinson's and stroke, and (5) the tumor necrosis factor protein putatively linked to obesity and diabetes. Appropriate protein targets include also, but are not limited to, enzymes catalyzing the following types of reactions: (1) oxido-reductases, (2) transferases, (3) hydrolases, (4) lyases, (5) isomerases, and (6) ligases.

The arachidonic acid pathway constitutes one of the main mechanisms for the production of pain and inflammation. The pathway produces different classes of end products, including the prostaglandins, thromboxane and leukotrienes.

Prostaglandins, an end product of cyclooxygenase metabolism, modulate immune function, mediate vascular phases of inflammation and are potent vasodilators. The major therapeutic action of aspirin and other non-steroidal anti-inflammatory drugs (NSAIDs) is proposed to be inhibition of the enzyme cyclooxygenase (COX). Anti-inflammatory potencies of different NSAIDs have been shown to be proportional to their action as COX inhibitors. It has also been shown that COX inhibition produces toxic side effects such as erosive gastritis and renal toxicity. The knowledge base regarding the toxic side effects of COX inhibitors has been gained through years of monitoring human therapies and human suffering. Two kinds of COX enzymes are now known to exist, with inhibition of COX 1 related to toxicity, and inhibition of COX2 related to reduction of inflammation. Thus, selective COX2 inhibition is a desirable characteristic of new anti-inflammatory drugs. The method of the invention can provide a route from identification of potential drug targets to validating these targets (for example, COX1 and COX2) as playing a role in disease (pain and inflammation) to an examination of the phenotype for the inhibition of one or both target isozymes without human suffering. Importantly, this information can be collected in vivo.

As an alternative strategy, the method of the invention can be used to define the phenotype of “genes of unknown function” obtained from various human genome sequencing projects or to assess the phenotype resulting, from inhibition of one isozyme subtype or one member of a family of related protein targets.

Definitions

Target: (also, “target component of a cell,” or “target cell component”) a constituent of a cell which contributes to and is necessary for the production or maintenance of a phenotype of the cell in which it is found. A target can be a single type of molecule or can be a complex of molecules. A target can be the product of a single gene, but can also be a complex comprising more than one gene product (for example, an enzyme comprising alpha and beta subunits, mRNA, tRNA, ribosomal RNA or a ribonucleoprotein particle such as a snRNP). Targets can be the product of a characterized gene (gene of known function) or the product of an uncharacterized gene (gene of unknown function).

Target Validation: the process of determining whether a target is essential to the maintenance of a phenotype of the cell type in which the target normally occurs.

For example, for pathogenic bacteria, researchers developing antimicrobials want to know if a compound which is potentially an antimicrobial agent not only binds to a target in vitro, but also binds to, and modulates the function of, a target in the bacteria in vivo, and especially under the conditions in which the bacteria are producing an infection—those conditions under which the antimicrobial agent must work to inhibit bacterial growth in an infected animal or human. If such compounds can be found that bind to a target in vitro and alter the target's function in cells resulting in an altered phenotype, as found by testing cells in culture and/or as found by testing cells in an animal, then the target is validated.

Phenotypic Effect: a change in an observable characteristic of a cell which can include, e.g., growth rate, level or activity of an enzyme produced by the cell, sensitivity to various agents, antigenic characteristics, and level of various metabolites of the cell. A phenotypic effect can be a change away from wild type (normal) phenotype, or can be a change towards wild type phenotype, for example. A phenotypic effect can be the causing or curing of a disease state, especially where mammalian cells are referred to herein. For cells of a pathogen or tumor cells, especially, a phenotypic effect can be the slowing of growth rate or cessation of growth.

Biomolecule: a molecule which can be produced as a gene product in cells that have been appropriately constructed to comprise one or more genes encoding the biomolecule. Production of the biomolecule can be turned on, when desired, by an inducible promoter. A biomolecule can be a peptide, polypeptide, or an RNA or RNA oligonucleotide, a DNA or DNA oligonucleotide, but is preferably a peptide. The same biomolecules can also be made synthetically. For peptides, see Merrifield, J., J. Am. Chem. Soc. 85: 2140-2154 (1963). For instance, an Applied Biosystems 431 A Peptide Synthesizer (Perkin Elmer) can be used for peptide synthesis. Biomolecules produced as gene products intracellularly are tested for their interaction with a target in the intracellular steps described herein (tests performed with cells in culture and tests performed with cells that have been introduced into animals). The same biomolecules produced synthetically are tested for their binding to an isolated target in an initial in vitro method described herein.

Synthetically produced biomolecules can also be used for a final step of the method for finding compounds that are competitive binders of the target.

Biomolecular Binder (of a target): a biomolecule which has been tested for its ability to bind to an isolated target cell component in vitro and has been found to bind to the target.

Biomolecular Inhibitor of Growth: a biomolecule which has been tested for its ability to inhibit the growth of cells constructed to produce the biomolecule in an “in culture” test of the effect of the biomolecule on growth of the cells, and has been found, in fact, to inhibit the growth of the cells in this test in culture.

Biomolecular Inhibitor of Infection: a biomolecule which has been tested for its ability to ameliorate the effects of infection, and has been found to do so. In the test, pathogen cells constructed to regulably express the biomolecule are introduced into one or more animals, the gene encoding the biomolecule is regulated so as to allow production of the biomolecule in the cells, and the effects of production of the biomolecule are observed in the infected animals compared to one or more suitable control animals.

Isolated: term used herein to indicate that the material in question exists in a physical milieu distinct from that in which it occurs in nature. For example, an isolated target cell component of the invention may be substantially isolated with respect to the complex cellular milieu in which it naturally occurs. The absolute level of purity is not critical, and those skilled in the art can readily determine appropriate levels of purity according to the use to which the material is to be put.

In many circumstances the isolated material will form part of a composition (for example, a more or less crude extract containing other substances), buffer system or reagent mix. In other circumstances, the material may be purified to essential homogeneity, for example as determined by PAGE or column chromatography (for example, HPLC).

Pathogen or Pathogenic Organism: an organism which is capable of causing disease, detectable by signs of infection or symptoms characteristic of disease. Pathogens can include prokaryotes (which include, for example, medically significant Gram-positive bacteria such as Streptococcus pneumoniae, Enterococcus faecalis and Staphylococcus aureus, Gram-negative bacteria such as Escherichia coli, Pseudomonas aeroginosa and Klebsiella pneumoniae, and “acid-fast” bacteria such as Mycobacteria, especially M. tuberculosis), eukaryotes such as yeast and fungi (for example, Candida albicans and Aspergillus fumigatus) and parasites. It should be recognized that pathogens can include such organisms as soil-dwelling organisms and “normal flora” of the skin, gut and orifices, if such organisms colonize and cause symptoms of infection in a human or other mammal, by abnormal proliferation or by growth at a site from which the organism cannot usually be cultured.

Methods for Simultaneously Identifying Individual Proteins in Complex Mixtures of Biological Molecules

The invention provides methods for simultaneously identifying individual proteins in complex mixtures of biological molecules and quantifying the expression levels of those proteins, e.g., proteome analyses. The methods compare two or more samples of proteins, one of which can be considered as the standard sample and all others can be considered as samples under investigation. The proteins in the standard and investigated samples are subjected separately to a series of chemical modifications, i.e., differential chemical labeling, and fragmentation, e.g., by proteolytic digestion and/or other enzymatic reactions or physical fragmenting methodologies. The chemical modifications can be done before, or after, or before and after fragmentation/ digestion of the polypeptide into peptides.

Peptides derived from the standard and the investigated samples are labeled with chemical residues of different mass, but of similar properties, such that peptides with the same sequence from both samples are eluted together in the separation procedure and their ionization and detection properties regarding the mass spectrometry are very similar. Differential chemical labeling can be performed on reactive functional groups on some or all of the carboxy-and/or amino-termini of proteins and peptides and/or on selected amino acid side chains. A combination of chemical labeling, proteolytic digestion and other enzymatic reaction steps, physical fragmentation and/or fractionation can provide access to a variety of residues to general different specifically labeled peptides to enhance the overall selectivity of the procedure.

The standard and the investigated samples are combined, subjected to multidimensional chromatographic separation, and analyzed by mass spectrometry methods. Mass spectrometry data is processed by special software, which allows for identification and quantification of peptides and proteins.

Depending on the complexity and composition of the protein samples, it may be desirable, or be necessary, to perform protein fractionation using such methods as size exclusion, ion exchange, reverse phase, or other methods of affinity purifications prior to one or more chemical modification steps, proteolytic digestion or other enzymatic reaction steps, or physical fragmentation steps.

The combined mixtures of peptides are first separated by a chromatography method, such as a multidimensional liquid chromatography, system, before being fed into a coupled mass spectrometry device, such as a tandem mass spectrometry device. The combination of multidimensional liquid chromatography and tandem mass spectrometry can be called “LC-LC-MS/MS.” LC-LC-MS/MS was first developed by Link A. and Yates J. R., as described, e.g., by Link (1999) Nature Biotechnology 17:676-682; Link (1999) Electrophoresis 18:1314-1334.

In practicing the methods of the invention, proteins can be first substantially or partially isolated from the biological samples of interest. The polypeptides can be treated before selective differential labeling; for example, they can be denatured, reduced, preparations can be desalted, and the like. Conversion of samples of proteins into mixtures of differentially labeled peptides can include preliminary chemical and/or enzymatic modification of side groups and/or termini; proteolytic digestion or fragmentation; post-digestion or post-fragmentation chemical and/or enzymatic modification of side groups and/or termini.

The differentially modified polypeptides and peptides are then combined into one or more peptide mixtures. Solvent or other reagents can be removed, neutralized or diluted, if desired or necessary. The buffer can be modified, or, the peptides can be redissolved in one or more different buffers, such as a “MudPIT” (see below) loading buffer. The peptide mixture is then loaded onto chromatography column, such as a liquid chromatography column, a 2D capillary coluni or a multidimensional chromatography column, to generate an eluate.

The eluate is fed into a mass spectrograph, such as a tandem mass spectrograph. In one aspect, an LC ESI MS and MS/MS analysis is complete. Finally, data output is processed by appropriate software using database searching and data analysis.

In practicing the methods of the invention, high yields of peptides can generated for mass spectrograph analysis. Two or more samples can be differentially labeled by selective labeling of each sample. Peptide modifications, i.e., labeling, are stable. Reagents having differing masses or reactive groups can be chosen to maximize the number of reactive groups and differentially labeled samples, thus allowing for a multiplex analysis of sample, polypeptides and peptides. In one aspect, a “MudPIT” protocol is used for peptide analysis, as described herein. The methods of the invention can be fully automated and can essentially analyze every protein in a sample.

High Throughput, Comparative Proteome Characterization

The invention provides high throughput, comparative proteome characterization. The invention provides a broad-based method for global profiling protein expression, which is a combination of differential peptides labeling, multi-dimensional chromatography coupled with mass spectrometry for separation, identification and quantification. Proteins are identified in complex mixtures with rapid speed, high sensitivity and accurate quantitative information. Using sets of labeling tags and modification methods, protein are differentially and efficiently modified with stable and flexible labeling. Second, by combination with multidimensional Liquid Chromatography (LC) and tandem mass spectrometry, the invention provides methods accurate and sensitive comparative proteomics in complex systems.

The invention provides a method for high throughput, comparative proteome characterization. The goal is to provide a broad-based method for global profiling protein expression, which is a combination of differential peptides labeling, multi-dimensional chromatography coupled with mass spectrometry for separation, identification and quantification. This method significantly improves over traditional methods. Proteins are identified in complex mixture with rapid speed, high sensitivity and accurate quantitative information.

First, by designing a set of labeling tags and modification methods, the invention provides novel approaches for modifying proteins differentially and efficiently with stable and flexible labeling. Second, by combination with multidimensional Liquid Chromatography (LC) and tandem mass spectrometry, the methods provide the speed and sensitivity for accurate comparative proteomics in complex systems. In alternative aspects, invention provides:

Differential peptide labeling

Compare various modifications and identify the top candidate(s)

Optimize reaction conditions for desired peptide/protein modification

Method validation

Optimize Multi-dimensional Protein Identification Technique MudPIT) procedure for high throughput differential proteome profiling

Reliable protein preparation

Optimize peptide separation and analysis

Method validation on model protein mixtures

The invention provides a high throughput proteomics technology with high speed, high efficiency and accurate quantitation, which can be employed for quantitative analysis of global protein expression in complex samples, and the detection and quantitation of specific proteins in complex samples.

An exemplary high throughput, comparative proteomics method uses a model pathway study of Streptomyces diversa (S. diversa).

The use of mass spectrometry to identify proteins whose sequences are present in either DNA or protein databases is well established and integrated to the field of Proteomics. One goal of Proteomics is to define the expressed proteins associated with a given cellular state, and another goal is to quantify changes in protein expression between cellular states. Many techniques have been developed to achieve these goals (see below). The present invention provides a non-gel based method of identifying individual proteins in complex protein mixtures simultaneously and quantifying protein expression level globally. It overcomes the limitations inherent in traditional techniques.

Comparative Proteomics Techniques

2D gel electrophoresis (2D GE) is the most commonly used technique in proteomics. In 2D GE, proteins are separated by isoelectric focusing according to their PI difference in the first dimension and by electrophoresis mobility according to their molecular weight difference in the second dimension. Separated proteins are usually visualized by staining. Quantitation is achieved by comparing the spot density. For spot identification, the method involves spot cutting, in gel digestion and peptide extraction. The next stage is analyzing these peptides using mass spectrometry or tandem mass spectrometry and database searching for identifications. The disadvantages of 2D GE approach are that it is very time consuming and labor intensive, and it does not work well for hydrophobic proteins, proteins with extreme pI, and non-abundant proteins.

Isotope-coded affinity tag (ICAT) is one of the new non-gel based methodologies that have a great impact on proteome research¹. The method is based on a newly synthesized class of chemical reagents (ICAT) used in combination with tandem mass spectrometry. The ICAT reagent contains a biotin affinity tag and a thiol specific reactive group (cysteine side chain), which are joined by a spacer domain available in two forms: regular (light), and isotopically heavy which includes eight deuterium atoms. First, a reduced protein mixture representing one cell state is derivatized with the isotopically light version of the ICAT reagent, while the corresponding reduced protein mixture representing a second cell state is derivatized with the isotopically heavy version of the ICAT reagent. Second, the labeled samples are combined and proteolytically digested to produce peptide fragments. Third, the tagged cysteine containing peptide fragments are isolated by avidin affinity chromatography. Finally, the isolated tagged peptides are separated and analyzed by microcapillary tandem mass spectrometry.

There are, however, limitations associated with this approach: (i) Differential labeling reagents rely on stable isotopes which are expensive and not very flexible to multiplex differential labeling; (ii) The moieties attached to the original peptides are approximately 500 Dalton heavy, which is heavier than some peptides and is likely to affect peptide ionization and fragmentation process; (iii) Some bonds in the labeling reagent are weak compared to the amide bond, which might complicate the MS/MS spectrum; (iv) Protein expression profiling is limited to duplex comparison; (v) The affinity interaction between biotin and avidin is too strong to release the immobilized peptide efficiently; (vi) the efficiency of protein reduction and alkylation are usually low; (vii) Some proteins do not contain cysteines so they are not going to be labeled.

Differential isotopic labeling of peptides for global quantification of proteins² is another method used currently, in which two different protein mixtures for quantitative comparison were digested to peptide mixtures. The peptide mixtures were separately methylated using either d0-or d3-methanol, the mixtures of methylated peptide were combined, and subjected to microcapillary HPLC-MS/MS. Parent proteins of methylated peptides were identified by correlative database searching of fragment ion spectra using SEQUEST or automated de novo sequencing that compared all tandem mass spectra of d0-and d3-methylated peptide ion pairs. Ratios of proteins in the two original mixtures were calculated by normalization of the area under the curve for d0-to d3-methylated peptide pairs.

There are several limitations specific to this approach: (i) differential labeling reagents relied on stable isotopes which are expensive and not flexible to differential labeling of more than two mixtures of peptides; (ii) labeling methods are limited only to methylation of c-terminal; (iii) protein expression profiling is limited to duplex comparison; (iv) one dimensional capillary HPLC chromatography was employed to separate peptides, which doesn't have enough capacity and resolving power for complex mixtures of peptides.

The invention overcomes the shortcomings of the currently available quantitative proteomics methods described above. The technology of the present method has speed, high efficiency and accurate quantitation, which is employed for quantitative analysis of global protein expression in complex samples. The basic approach described is employed for: (i) quantitative analysis of global protein expression in complex samples (such as cells, tissues, fractions and etc.), (ii) the detection and quantitation of specific proteins in complex samples, and (iii) quantitative measurement of specific enzymatic activities in complex samples.

Novelties of this approach include: (i) design of differential labeling reagents for peptides and methods for efficient peptide modification; (ii) multiplex analysis; (iii) combination of labeling by chemical modifications of termini and/or side chains of peptides; (iv) combination of chemical modification and proteolytic digestions in order to achieve the most favorable and selective chemical modification of peptides; (v) improvement of multidimensional chromatography for better protein/peptide separation and identification.

Experimental Design and Methods

The present application provides a non-gel based method of identifying individual proteins in complex protein mixtures simultaneously and quantifying protein expression level globally. It overcomes the limitations inherent in traditional techniques.

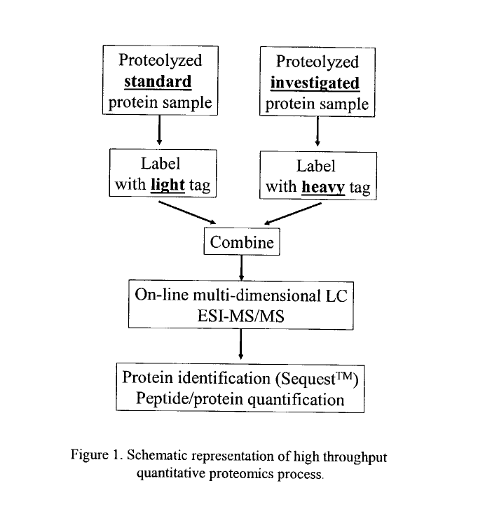

In detail, two or more samples of proteins are compared, one of which is considered as the standard sample and all others are considered as samples under investigation. First, the proteins in the standard and investigated samples are subjected to a sequence of proteolytic digestion and/or other enzymatic reaction in separate tubes. Then, these digested peptides are modified (novel differential chemical labeling). Peptides derived from the standard and the investigated samples are labeled with chemical residues of different mass, but they have similar properties such that the differential labeled peptides are eluted together in the separation procedure and their ionization and fragmentation properties regarding the mass spectrometry are very similar. Next, the samples are combined, separated by multidimensional chromatography, and analyzed by mass spectrometry methods. Finally, mass spectrometry data is processed by special software, for identification and quantification of proteins. This procedure is schematically illustrated in FIG. 1. Differential characterization of post-translationally modified proteins is achieved by combining affinity separation techniques for enrichment of the modified proteins or special MS monitoring or data analysis with above approaches.

Differential Peptide Labeling

Differential chemical labeling is performed on reactive functional groups on the termini of proteins and peptides and/or on the side chains of amino acids. A combination of chemical labeling, proteolytic digestion, and other enzymatic reaction steps can provide access to a variety of specifically labeled peptides, which enhances the overall selectivity of the procedure. The combined mixtures of peptides are separated by improving a current chromatography method called Multi-dimensional Protein Identification Technique (MudPIT)³.

a. Chemical Transformations Involved in Differential Labeling:

(1) Esterification of C-termini of the peptides and carboxylic acid groups in the side chains; (2) Amidation of C-termini of the peptides and carboxylic acid groups in the side chains; (might require protection of amine groups first); (3) Acylation of N-termini of the peptides and amino and hydroxyl groups in the side chains.

The esterification, amidation, and acylation reactions are performed on the mixtures of peptides in a fashion similar to other reactions of the types already described in previous part, or modified as needed in each particular case.

b. Reagents for Differential Labeling:

Mixtures of peptides coming from the standard protein samples and the investigated protein samples are labeled separately with differential reagents. These differential reagents differ in molecular mass, but do not differ in retention properties regarding the separation method used and in ionization and detection properties regarding the mass spectrometry methods used. Thus, these differential reagents differ either in their isotope composition (isotopical reagents) or they differ structurally by a rather small fragment, which change does not alter the properties stated above (homologous reagents). The obvious choices for such reagents are aliphatic alcohols, aliphatic amines, and aliphatic acids. Isotopic reagents based on aliphatic alcohols, amines, or acids contain different amount of protons and deuterons in different reagents, e.g., CH₃CH₂OH and CD₃CD₂OH (mass difference is 5 Da) or CH₃CH₂CO₂H and CD₃CD₂CO₂H (mass difference is 5 Da). The homologous reagents differ from each other by the number of CH₂ moieties in their molecules, e.g., CH₃OH and CH₃CH₂OH (mass difference is 14 Da) or CH₃CO₂H and CH₃CH₂CO₂H (mass difference is 14 Da).

The alcohol reagents esterify peptide C-terminals and/or Glu and Asp side chains, the amines form amide bond with peptide C-terminals and/or Glu and Asp side chains, and the acids form amide bond with peptide N-terminals and/or Lys and Arg side chains. Substituents may be introduced into the mass-labeling reagents in order to tune their retention, ionization, and detection properties.

Differential Labeling Progress:

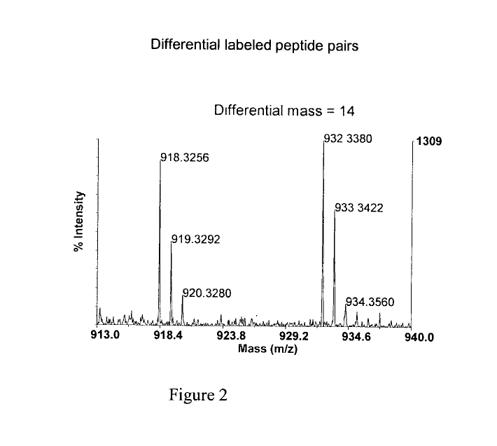

The peptide esterification is performed using different alcohols. Labeling process has been optimized. FIG. 2 shows one example: a peptide is differential labeled by one of the homologous reagent pairs. In this case: methanol and ethanol. The physical/chemical properties of those differential labeled peptide pairs was further tested, and it was found that they are very similar in terms of reverse phase LC elution and ionization efficiency. Differential labeled peptide pairs with a methyl group difference serve as ideal mutual internal standards for quantification. Advantages of this approach include the minimum cost of the reagents, the straight forward labeling procedure, and high product yield. All the other homologous and isotope reagents are tested and the best one for proteomics application is chosen.

FIG. 2 is an illustration of a MALDI MS spectrum of a peptide pairs. These peptides are differentially esterified by either methanol or ethanol. They have the identical sequence before the labeling.

Methods for Peptide/Protein Separation, Detection and Analysis:

a. Peptide Separation and Detection

The cutting edge methodology that represents a significant step forward in proteome analysis is the use of multidimensional liquid chromatography coupled to tandem mass spectrometry (LC-LC-MS/MS), which was first developed by Link A. and Yates J. R.^{4,5,6} and further improved by Washburn M., Wolters D., and Yates J. R.³. The existence and further improvement of this technique are critical factors in the present approach for the application of complex peptide separation and full automation, which makes it the most ideal technology for high throughput proteomics. MudPIT has been previously reported in various incarnations involving reversed phase columns coupled to either cation exchange columns⁷ or size exclusion columns⁸. However, it was only when the technique was employed with a mixed bed microcapillary column containing strong cation exchange (SCX) and reversed phase chromatography (RPC) resins that the true utility of MudPIT was demonstrated. First, a denatured and reduced protein mixture is digested with trypsin to produce peptide fragments. The mixture is loaded onto a microcapillary column containing SCX resin upstream of RPC resin, eluting directly into a tandem mass spectrometer. A discrete fraction of the absorbed peptides are displaced from the SCX column onto the RPC column using a step gradient of salt, causing the peptides to be retained on the RPC column while contaminating salts and buffers are washed through. Peptides are then eluted from the RPC column using an acetonitrile gradient, and analyzed by MS/MS. This process is repeated using increasing salt concentration to displace additional fractions from the SCX column. This is applied in an iterative manner, typically involving 10-20 steps, and the MS/MS data from all of the fractions are analyzed by database searching^{9,10} and combined to give an overall picture of the protein components present in the initial sample. The MudPIT technique can be run in a fully automated system. The use of two dimensions for chromatographic separation also greatly increases the number of peptides that can be identified from very complex mixtures. In one typical 14 step MudPIT run, there are up to 1,000 proteins can be identified with high confidence. In order to identify more proteins from complex protein samples, one has to reduce protein complexity must be reduced prior to proteolysis by pre-fractionation using techniques such as size exclusion, ion exchange, reverse phase, or all the possible affinity purifications.

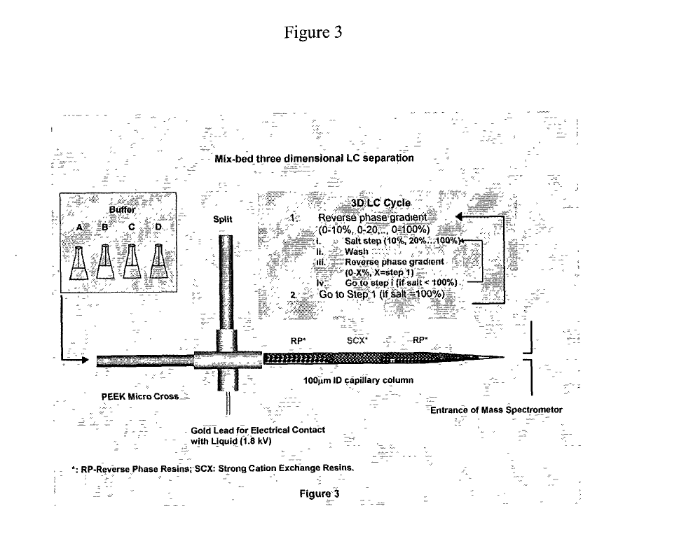

Instead of using any of the pre-fractionation technique above, the present application proposes to improve MudPIT technique by employing a three-dimensional microcapillary column containing reversed phase (RPC), strong cation exchange (SCX) and reversed phase (RPC). First, a denatured and reduced protein mixture is digested with trypsin to produce peptide fragments. Without desalting, the mixture is directly loaded onto a microcapillary column containing RPC resin, SCX resin and RPC resin, accordingly, eluted directly into a tandem mass spectrometer. A discrete fraction of the absorbed peptides are displaced from the first RPC to the SCX section using a reverse phase gradient (0-X %). This fraction of peptides are retained onto SCX section and then sub-fractionated from the SCX column onto the RPC column using a step gradient of salt, causing part of the peptides to be eluted and retained on the last RPC section while contaminating salts and buffers are washed through. Peptides are then eluted from the RPC column using the same reverse phase gradient (0-X %), and analyzed by MS/MS. This process is repeated using increasing salt concentration to displace additional sub-fractions from the SCX column following each step by a reverse phase gradient. Once the completion of the whole sequence of salt steps, next cycle begins with a higher reverse phase gradient (0-Y %, Y>X). Each cycle is applied in an iterative manner, depends on the complexity of the peptides, involving 3-6 acetonitrile cycles followed by 5-10 salt steps, and the MS/MS data from all of the fractions are analyzed by database searching. FIG. 3 illustrates 3D LC set-up and process.

3D LC MS is a fully automated technique using LC in combination with mass spectrometry and database search for highly complex mixtures. It is competitive toward the 2D GE technique in the following terms. It is universal, identifies proteins with extremes in pI, MW, and wide variety of protein classes. It can access hydrophobic proteins. It has high sensitivity, peak capacity and gives dynamic range greater than 10,000 to 1. It is time and labor efficient with its automatic workflow.

3D LC plays an important role on both qualitative proteomics as well as quantitative proteomics with the combination of novel tagging method.

b. Sequence Analysis and Quantification:

Both quantity and sequence identity of the protein from which the modified peptide originated is determined by multistage MS. This is achieved by the operation of the mass spectrometer in a dual mode in which it alternates in successive scans between measuring the relative quantities of peptides eluting from the capillary column and recording the sequence information of selected peptides. Peptides are quantified by measuring in the MS mode the relative signal intensities for pairs or series of peptide ions of identical sequence that are tagged differentially, which therefore differ in mass by the mass differential encoded within the differential labeling reagents. Peptide sequence information is automatically generated by selecting peptide ions of a particular mass-to-charge (m/z) ratio for collision-induced dissociation (CID) in the mass spectrometer operating in the tandem MS mode^{6,11,12}. The resulting tandem mass spectra is correlated to sequence databases to identify the protein from which the sequenced peptide originated. Commercial available software that may be used is Turbo SEQUEST by Thermofinnigan, Mascot by Matrix Science, and Sonar MS/MS by Proteometrics. Special software development will be developed for automated relative quantification.

The present application provides a non-gel based method of identifying individual proteins in complex protein mixtures simultaneously and quantifying protein expression level globally. It overcomes the limitations inherent in traditional techniques.

Literature Cited

1. Gygi, Steven P.; Rist, Beate; Gerber, Scott A.; Turecek, Frantisek; Gelb, Michael H.; Aebersold, Ruedi. Quantitative analysis of complex protein mixtures using isotope-coded affinity tags. In: Nature Biotechnology October 1999. 17 (10): 994-999.

2. Goodlett, David R.; Keller, Andrew; Watts, Julian D.; Newitt, Richard; Yi, Eugene C.; Purvine, Samuel; Eng, Jimmy K.; von Haller, Priska; Aebersold, Ruedi; Kolker, Eugene. Differential stable isotope labeling of peptides for quantitation and de novo sequence derivation. In: Rapid Communications in Mass Spectrometry 2001. 15 (14): 1214-1221.

3. Washburn, Michael P.; Wolters, Dirk; Yates, John R.,. Large-scale analysis of the yeast proteome by multidimensional protein identification technology. In: Nature Biotechnology Mar., 2001. 19 (3): 242-247.

4. Yates, J. R.; Link, Andrew J.; Schieltz, David A.; Eng, Jimmy K.; Carmack, Edwin American Societies for Experimental Biology. (Annual Meeting of the American Societies for Experimental Biology on Biochemistry and Molecular Biology 99 San Francisco, Calif., USA May 16-20, 1999). Mining proteomes using mass spectrometry: New approaches to help define function. In: FASEB Journal Apr. 23, 1999. 13 (7): A1431.

5. Link, Andrew J.; Robison, Keith; Church, George M.. Comparing the predicted and observed properties of proteins encoded in the genome of Escherichia coli K-12. In: Electrophoresis 1997. 18 (8): 1259-1313.

6. Link, Andrew J.; Hays, Lara G.; Carmack, Edwin B.; Yates, John R.,. Identifying the major proteome components of Haemophilus influenzae type-stain NCTC 8143. In: Electrophoresis 1997. 18 (8): 1314-1334.

7. Rose, Donald J.; Opiteck, Gregory J.. Two-dimensional gel electrophoresis/liquid chromatography for the micropreparative isolation of proteins. In: Analytical Chemistry 1994. 66 (15): 2529-2536.

8. Opiteck, Gregory J.; Ramirez, Suzanne M.; Jorgenson, James W.; Moseley, M. Arthur,. Comprehensive two-dimensional high-performance liquid chromatography for the isolation of over expressed proteins and proteome mapping. In: Analytical Biochemistry May 1, 1998. 258 (2): 349-361.

9. Yates, J R 3rd; Eng, J K; McCormack, A L. Mining genomes: correlating tandem mass spectra of modified and unmodified peptides to sequences in nucleotide databases. Analytical Chemistry Sep. 15, 1995 , 67(18):3202-10.

10. Yates, J R 3d; Eng, J K; McCormack, A L; Schieltz, D. Method to correlate tandem mass spectra of modified peptides to amino acid sequences in the protein database. Analytical Chemistry Apr. 15, 1995 , 67(8):1426-36.

11. Gygi, S P; Rist, B; Gerber, S A; Turecek, F; Gelb, M H; Aebersold, R. Quantitative analysis of complex protein mixtures using isotope-coded affinity tags. Nature Biotechnology October 1999 17(10):994-9.

12. Gygi, S P; Rochon, Y; Franza, B R; Aebersold, R. Correlation between protein and mRNA abundance in yeast. Molecular and Cellular Biology March 1999 19(3):1720-30.

### EXAMPLES

The following examples are offered to illustrate, but not to limit the claimed invention.

### Example 1

### Identifying Proteins by Differential Labeling of Peptides

An exemplary method for identifying proteins by differential labeling of peptides is provided, as described below.

First, a denatured and reduced protein mixture is digested with trypsin to produce peptide fragments. The mixture is loaded onto a microcapillary column containing a sulfonated styrene resin (e.g., SCX resin, as from Dionex Corporation, Sunnyvale, Calif.) upstream of RPC resin (Rapid Prototyping Chemicals, Switzerland), eluting directly into a tandem mass spectrometer. A discrete fraction of the absorbed peptides are displaced from the SCX column onto the RPC column using a step gradient of salt, causing the peptides to be retained on the RPC column while contaminating salts and buffers are washed through. Peptides are then eluted from the RPC column using an acetonitrile gradient, and analyzed by MS/MS. This process is repeated using increasing salt concentration to displace additional fractions from the SCX column. This is applied in an iterative manner; it can be repeated 10 to 20, or more, times.

The MS/MS data from all of the fractions are analyzed by database searching, as described, for example, by Yates, J. R., III, et al (1995) Anal. Chem. 67, 1426-1436; Eng, J. et al (1994) J. Amer. Mass Spectrom. 5, 976-989. The data are combined to give an overall picture of the protein components present in the initial sample. The MudPIT technique can be run in a fully automated system. The use of two dimensions for chromatographic separation also greatly increases the number of peptides that can be identified from very complex mixtures.

### Example 2

### Identifying Proteins by Differential Labeling of Peptides

An exemplary method for synthesizing a differential labeling reagent is provided, as described below.

The invention provides chimeric labeling reagents comprising biotin and an amino acid reactive moiety, such as succimide, isothiocyanate, isocyanate. The amino acid reactive moiety can be attached directly or indirectly (i.e., through a linker) to the biotin. The biotin can comprise up to 6 deuterium atoms or six hydrogen atoms. Alternatively, other isotopes, such a 13C, 18O, as described above, can be incorporated either into the biotin moiety, the amino acid reactive moiety or the crosslinker moiety. The biotin facilitates purification, see, e.g., WO 00/11208, and, by comprising at least one isotope, simultaneously allows mass discrimination in the mass spectrometer. The activated group allows covalent bonding to amino acids, such as lysines or cysteines.

An exemplary precursor to biotin that can be used is:

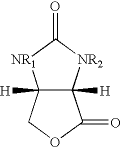

A Grignard reaction is performed with the following compound:

XMg-(CD2)4-MgX,

where X is chlorine or bromine. The reaction is similar to the one described in U.S. Pat. No. 4,876,350, which describes the chemical synthesis of regular biotin.

A deuteurated and undeuteurated biotin, subsequently derivatized to a pentafluorophenyl ester, can then be attached to iodoacetic acid anhydride or as an NHS ester, or other amino acid reactive groups. For example,

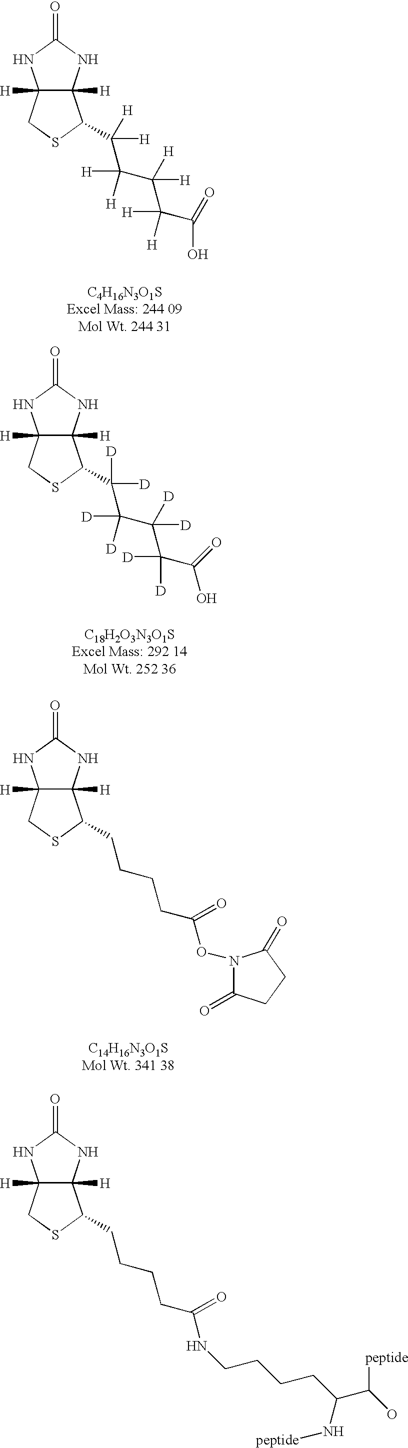

This technology allows the direct comparison between two differential proteome samples. For example, protein samples are differentially tagged with the isotope-coded affinity tags of the invention. These tags are only distinguishable by having different isotope compositions. The isotope- (e.g., deuterium-) containing moiety can be the biotin, the linker or the amino acid reactive group, or any combination thereof. The biotin moiety facilitates purification of the peptides. An isotopically “heavy” and isotopically “light” tagged peptides are separately mixed with denatured differential protein samples. The tagged proteins are digested with a protease before or after mixing of samples. Tagged peptides are purified on an avidin column. The column is washed, and the tagged peptides eluted. After elution of the tagged peptides, the peptide mixture is separated using capillary chromatography and the peptide mass is determined. Peptide masses with the exact difference as the isotopic tag correspond to the identical peptide species and can be directly compared quantitatively.

A number of embodiments of the invention have been described. Nevertheless, it will be understood that various modifications may be made without departing from the spirit and scope of the invention. Accordingly, other embodiments are within the scope of the following claims.

What is claimed is:

1. A method for identifying proteins by differential labeling of peptides, the method comprising the following steps:
(a) providing a sample comprising a polypeptide;
(b) providing a plurality of labeling reagents which differ in molecular mass but have the same or nearly identical or similar chromatographic retention properties and that have the same or nearly identical or similar ionization and detection properties in mass spectrographic analysis, wherein the differences in molecular mass are distinguishable by mass spectrographic analysis;
(c) fragmenting the polypeptide into peptide fragments by enzymatic digestion or by non-enzymatic fragmentation;
(d) contacting the labeling reagents of step (b) with the peptide fragments of step (c), thereby labeling the peptides with the differential labeling reagents;
(e) separating the peptides by chromatography to generate an eluate;
(f) feeding the eluate of step (e) into a mass spectrometer and quantifying the amount of each peptide and generating the sequence of each peptide by use of the mass spectrometer;
(g) inputting the sequence to a computer program product which compares the inputted sequence to a database of polypeptide sequences to identify the polypeptide from which the sequenced peptide originated.

2. The method of claim 1, wherein the sample of step (a) comprises a cell or a cell extract.

3. The method of claim 1, further comprising providing two or more samples comprising a polypeptide.

4. The method of claim 3, wherein one sample is derived from a wild type cell and one sample is derived from an abnormal or a modified cell.

5. The method of claim 4, wherein the abnormal cell is a cancer cell.

6. The method of claim 1, further comprising purifying or fractionating the polypeptide before the fragmenting of step (c).

7. The method of claim 1, further comprising purifying or fractionating the polypeptide before the labeling of step (d).

8. The method of claim 1, further comprising purifying or fractionating the labeled peptide before the chromatography of step (e).

9. The method of claim 6, claim 8 or claim 8, wherein the purifying or fractionating comprises a method selected from the group consisting of size exclusion chromatography, size exclusion chromatography, HPLC, reverse phase HPLC and affinity purification.

10. The method of claim 1, further comprising contacting the polypeptide with a labeling reagent of step (b) before the fragmenting of step (c).

11. The method of claim 1, wherein the labeling reagent of step (b) comprises the general formulae selected from the group consisting of:
Z^{A}OH and Z^{B}OH, to esterify peptide C-terminals and/or Glu and Asp side chains;
Z^{A}NH₂ and Z^{B}NH₂, to form amide bond with peptide C-terminals and/or Glu and Asp side chains; and
Z^{A}CO₂H and Z^{B}CO₂H. to form amide bond with peptide N-terminals and/or Lys and Arg side chains;
wherein Z^{A} and Z^{B} independently of one another comprise the general formula R-Z¹-A¹-Z²-A²-Z³-A³-Z⁴-A⁴-,
Z¹, Z², Z³, and Z⁴ independently of one another, are selected from the group consisting of nothing, O, OC(O), OC(S), OC(O)O, OC(O)NR, OC(S)NR, OSiRR¹, S, SC(O), SC(S), SS, S(O), S(O₂), NR, NRR¹⁺, C(O), C(O)O, C(S), C(S)O, C(O)S, C(O)NR, C(S)NR, SiRR¹, (Si(RR¹)O)n, SNRR¹, Sn(RR¹)O, BR(OR¹), BRR¹, B(OR)(OR¹), OBR(OR¹), OBRR¹, and OB(OR)(OR¹), and R and R¹ is an alkyl group,
A¹, A², A³, and A⁴ independently of one another, are selected from the group consisting of nothing or (CRR¹)n, wherein R, R¹, independently from other R and R¹ in Z¹ to Z⁴ and independently from other R and R¹ in A¹ to A⁴, are selected from the group consisting of a hydrogen atom, a halogen atom and an alkyl group;
n in Z¹ to Z⁴, independent of n in A¹ to A⁴, is an integer having a value selected from the group consisting of 0 to about 51; 0 to about 41; 0 to about 31; 0 to about 21, 0 to about 11 and 0 to about 6.

12. The method of claim 11, wherein the alkyl group is selected from the group consisting of an alkenyl, an alkynyl and an aryl group.

13. The method of claim 11, wherein one or more C—C bonds from (CRR¹)n are replaced with a double or a triple bond,

14. The method of claim 13, wherein an R or an R¹ group is deleted.

15. The method of claim 13, wherein (CRR¹)n is selected from the group consisting of an o-arylene, an m-arylene and a p-arylene, wherein each group has none or up to 6 substituents.

16. The method of claim 13, wherein (CRR¹)n is selected from the group consisting of a carbocyclic, a bicyclic and a tricyclic fragment, wherein the fragment has up to 8 atoms in the cycle with or without a heteroatom selected from the group consisting of an O atom, a N atom and an S atom.

17. The method of claim 1, wherein two or more labeling reagents have the same structure but a different isotope composition.

18. The method of claim 11, wherein Z^{A} has the same structure as Z^{B}, but Z^{A} has a different isotope composition than Z^{B}.

19. The method of claim 17, wherein the isotope is boron-10 and boron-11.

20. The method of claim 17, wherein the isotope is carbon-12 and carbon-13.

21. The method of claim 17, wherein the isotope is nitrogen-14 and nitrogen-15.

22. The method of claim 17, wherein the isotope is sulfur-32 and sulfur-34.

23. The method of claim 17, wherein, where the isotope with the lower mass is x and the isotope with the higher mass is y, and x and y are integers, x is greater than y.

24. The method of claim 17, wherein x and y are between 1 and about 11, between 1 and about 21, between 1 and about 31, between 1 and about 41, or between 1 and about 51.

25. The method of claim 1, wherein the labeling reagent of step (b) comprises the general formulae selected from the group consisting of:
i. CD₃(CD₂)ₙOH/CH₃(CH₂)ₙOH, to esterify peptide C-terminals, where n=0, 1, 2or y;
ii. CD₃(CD₂)ₙNH₂/CH₃(CH₂)ₙNH₂, to form amide bond with peptide C-terminals, where n=0, 1, 2 or y; and
iii. D(CD₂)ₙCO₂H/H(CH₂)ₙCO₂H, to form amide bond with peptide N-terminals, where n=0, 1, 2 or y;
wherein D is a deuteron atom, and y is an integer selected from the group consisting of about 51; about 41; about 31; about 21, about 11; about 6 and between about 5 and 51.

26. The method of claim 1, wherein the labeling reagent of step (b) comprises the general formulae selected from the group consisting of:
i. Z^{A}OH and Z^{B}OH to esterify peptide C-terminals;
ii. Z^{A}NH₂/Z^{B}NH₂ to form an amide bond with peptide C-terminals; and
iii. Z^{A}CO₂H/Z^{B}CO₂H to form an amide bond with peptide N-terminals;
wherein Z^{A} and Z^{B} have the general formula R-Z¹-A¹-Z²-A²-Z³-A³-Z⁴-A⁴-Z, Z², Z³, and Z⁴, independently of one another, are selected from the group consisting of nothing, O, OC(O), OC(S), OC(O)O, OC(O)NR, OC(S)NR, OSiRR¹, S, SC(O), SC(S), SS, S(O), S(O₂), NR, NRR¹⁺, C(O), C(O)O, C(S), C(S)O, C(O)S, C(O)NR, C(S)NR, SiRR¹, (Si(RR¹)O)n, SNRR¹, Sn(RR¹)O, BR(OR¹), BRR¹, B(OR)(OR¹), OBR(OR¹), OBRR¹, and OB(OR)(OR¹);
A¹, A², A³, and A⁴, independently of one another, are selected from the group consisting of nothing and the general formulae (CRR¹)n, and,
R and R¹ is an alkyl group.

27. The method of claim 26, wherein a single C—C bond in a (CRR¹)n group is replaced with a double or a triple bond.

28. The method of claim 27, wherein R and R¹ are absent.

29. The method of claim 27, wherein (CRR)n comprises a moiety selected from the group consisting of an o-arylene, an m-arylene and a p-arylene, wherein the group has none or up to 6 substituents.

30. The method of claim 27, wherein the group comprises a carbocyclic, a bicyclic, or a tricyclic fragments with up to 8 atoms in the cycle, with or without a heteroatom selected from the group consisting of an O atom, an N atom and an S atom.

31. The method of claim 26, wherein R, R¹, independently from other R and R¹ in Z¹-Z⁴ and independently from other R and R¹ in A¹-A⁴, are selected from the group consisting of a hydrogen atom, a halogen and an alkyl group.

32. The method of claim 31, wherein the alkyl group is selected from the group consisting of an alkenyl, an alkynyl and an aryl group.

33. The method of claim 26, wherein n in Z¹-Z⁴ is independent of n in A¹-A⁴ and is an integer selected from the group consisting of about 51; about 41; about 31; about 21, about 11 and about 6.

34. The method of claim 26, wherein Z^{A} has the same structure a Z^{B} but Z^{A} further comprises x number of —CH₂— fragment(s) in one or more A¹-A⁴ fragments, wherein x is an integer.

35. The method of claim 26, wherein Z^{A} has the same structure a Z^{B} but Z^{A} further comprises x number of —CF₂— fragment(s) in one or more A¹-A⁴ fragments, wherein x is an integer.

36. The method of claim 26, wherein Z^{A} comprises x number of protons and Z^{B} comprises y number of halogens in the place of protons, wherein x and y are integers.

37. The method of claim 26, wherein Z^{A} contains x number of protons and Z^{B} contains y number of halogens, and there are x-y number of protons remaining in one or more A¹-A⁴ fragments, wherein x and y are integers

38. The method of claim 26, wherein Z^{A} further comprises x number of —O— fragment(s) in one or more A¹-A⁴ fragments, wherein x is an integer.

39. The method of claim 26, wherein Z^{A} further comprises x number of —S— fragment(s) in one or more A¹-A⁴ fragments, wherein x is an integer.

40. The method of claim 26, wherein Z^{A} further comprises x number of —O— fragment(s) and Z^{B} further comprises y number of —S— fragment(s) in the place of —O— fragment(s), wherein x and y are integers.

41. The method of claim 26, wherein Z^{A} further comprises x-y number of —O— fragment(s) in one or more A¹-A⁴ fragments, wherein x and y are integers.

42. The method of claim 37, claim 40 or claim 41, wherein x and y are integers selected from the group consisting of between 1 about 51; between 1 about 41; between 1 about 31; between 1 about 21, between 1 about 11 and between 1 about 6, wherein x is greater than y.

43. The method of claim 1, wherein the labeling reagent of step (b) comprises the general formulae selected from the group consisting of:
i. CH₃(CH₂)ₙOH/CH₃(CH₂)ₙ₊ᵣOH, to esterify peptide C-terminals, where n=0, 1, 2, . . . ,y;m=1, 2, . . . , y;
ii. CH₃(CH₂)ₙ NH₂/CH₃(CH₂)ₙ₊ₙNH₂, to form amide bond with peptide C-terminals, where n=0, 1, 2, . . . , y; m=1, 2, . . . , y; and,
iii. H(CH₂)ₙCO₂H/H(CH₂)ₙ₊ₘCO₂H, to form amide bond with peptide N-terminals, where n=0, 1, 2, . . . , y; m=1, 2, . . . , y;
wherein n, m and y are integers.

44. The method of claim 43, wherein n, m and y are integers selected from the group consisting of about 51; about 41; about 31; about 21, about 11; about 6 and between about 5 and 51.

45. The method of claim 1, wherein the separating of step (e) comprises a liquid chromatography system.

46. The method of claim 1, wherein the liquid chromatography system comprises a multidimensional liquid chromatography.

47. The method of claim 1, wherein the mass spectrometer comprises a tandem mass spectrometry device.

48. The method of claim 1, further comprising quantifying the amount of each polypeptide.

49. The method of claim 1, further comprising quantifying the amount of each peptide.

50. A method for defining the expressed proteins associated with a given cellular state, the method comprising the following steps:
(a) providing a sample comprising a cell in the desired cellular state;
(b) providing a plurality of labeling reagents which differ in molecular mass but do not differ in chromatographic retention properties and do not differ in ionization and detection properties in mass spectrographic analysis, wherein the differences in molecular mass are distinguishable by mass spectrographic analysis;
(c) fragmenting polypeptides derived from the cell into peptide fragments by enzymatic digestion or by non-enzymatic fragmentation;
(d) contacting the labeling reagents of step (b) with the peptide fragments of step (c), thereby labeling the peptides with the differential labeling reagents;
(e) separating the peptides by chromatography to generate an eluate;
(f) feeding the eluate of step (e) into a mass spectrometer and quantifying the amount of each peptide and generating the sequence of each peptide by use of the mass spectrometer;
(g) inputting the sequence to a computer program product which compares the inputted sequence to a database of polypeptide sequences to identify the polypeptide from which the sequenced peptide originated, thereby defining the expressed proteins associated with the cellular state.

51. A method for quantifying changes in protein expression between at least two cellular states, the method comprising the following steps:
(a) providing at least two samples comprising cells in a desired cellular state;
(b) providing a plurality of labeling reagents which differ in molecular mass but do not differ in chromatographic retention properties and do not differ in ionization and detection properties in mass spectrographic analysis, wherein the differences in molecular mass are distinguishable by mass spectrographic analysis;
(c) fragmenting polypeptides derived from the cells into peptide fragments by enzymatic digestion or by non-enzymatic fragmentation;
(d) contacting the labeling reagents of step (b) with the peptide fragments of step (c), thereby labeling the peptides with the differential labeling reagents, wherein the labels used in one same are different from the labels used in other samples;
(e) separating the peptides by chromatography to generate an eluate;
(f) feeding the eluate of step (e) into a mass spectrometer and quantifying the amount of each peptide and generating the sequence of each peptide by use of the mass spectrometer;
(g) inputting the sequence to a computer program product which identifies from which sample each peptide was derived, compares the inputted sequence to a database of polypeptide sequences to identify the polypeptide from which the sequenced peptide originated, and compares the amount of each polypeptide in each sample, thereby quantifying changes in protein expression between at least two cellular states.

52. A method for identifying proteins by differential labeling of peptides, the method comprising the following steps:
(a) providing a sample comprising a polypeptide;
(b) providing a plurality of labeling reagents which differ in molecular mass but do not differ in chromatographic retention properties and do not differ in ionization and detection properties in mass spectrographic analysis, wherein the differences in molecular mass are distinguishable by mass spectrographic analysis;
(c) fragmenting the polypeptide into peptide fragments by enzymatic digestion or by non-enzymatic fragmentation;
(d) contacting the labeling reagents of step (b) with the peptide fragments of step (c), thereby labeling the peptides with the differential labeling reagents;
(e) separating the peptides by multidimensional liquid chromatography to generate an eluate;
(f) feeding the eluate of step (e) into a tandem mass spectrometer and quantifying the amount of each peptide and generating the sequence of each peptide by use of the mass spectrometer;
(g) inputting the sequence to a computer program product which compares the inputted sequence to a database of polypeptide sequences to identify the polypeptide from which the sequenced peptide originated.

53. A chimeric labeling reagent comprising
(a) a first domain comprising a biotin; and
(b) a second domain comprising a reactive group capable of covalently binding to an amino acid,
wherein the chimeric labeling reagent comprises at least one isotope.

54. The chimeric labeling reagent of claim 53, wherein the isotope is in the first domain.

55. The chimeric labeling reagent of claim 54, wherein the isotope is in the biotin.

56. The chimeric labeling reagent of claim 53, wherein the isotope is in the second domain.

57. The chimeric labeling reagent of claim 53, wherein the isotope is selected from the group consisting of a deuterium isotope, a boron-10 or boron-11 isotope, a carbon-12 or a carbon-13 isotope, a nitrogen-14 or a nitrogen-15 isotope and a sulfur-32 or a sulfur-34 isotope.

58. The chimeric labeling reagent of claim 53 comprising two or more isotopes.

59. The chimeric labeling reagent of claim 53, wherein the reactive group capable of covalently binding to an amino acid is selected from the group consisting of a succimide group, an isothiocyanate group and an isocyanate group.

60. The chimeric labeling reagent of claim 53, wherein the reactive group capable of covalently binding to an amino acid binds to a lysine or a cysteine.

61. The chimeric labeling reagent of claim 53, further comprising a linker moiety linking the biotin group and the reactive group.

62. The chimeric labeling reagent of claim 53, wherein the linker moiety comprises at least one isotope.

63. The chimeric labeling reagent of claim 53, wherein the linker is a cleavable moiety.

64. The chimeric labeling reagent of claim 53, wherein the linker can be cleaved by enzymatic digest.

65. The chimeric labeling reagent of claim 53, wherein the linker can be cleaved by reduction.

66. A method of comparing relative protein concentrations in a sample comprising
(a) providing a plurality of differential small molecule tags, wherein the small molecule tags are structurally identical but differ in their isotope composition, and the small molecules comprise reactive groups that covalently bind to cysteine or lysine residues or both;
(b) providing at least two samples comprising polypeptides;
(c) attaching covalently the differential small molecule tags to amino acids of the polypeptides;
(d) determining the protein concentrations of each sample in a tandem mass spectrometer; and,
(d) comparing relative protein concentrations of each sample.

67. The method of claim 66, wherein the sample comprises a complete or a fractionated cellular sample.

68. The method of claim 66, wherein differential small molecule tags comprise a chimeric labeling reagent comprising (a) a first domain comprising a biotin; and, (b) a second domain comprising a reactive group capable of covalently binding to an amino acid, wherein the chimeric labeling reagent comprises at least one isotope.

69. The method of claim 68, wherein the isotope is selected from the group consisting of a deuterium isotope, a boron-10 or boron-11 isotope, a carbon-12 or a carbon-13 isotope, a nitrogen-14 or a nitrogen-15 isotope and a sulfur-32 or a sulfur-34 isotope.

70. The method of claim 68, wherein the chimeric labeling reagent comprises two or more isotopes.

71. The method of claim 68, wherein the reactive group capable of covalently binding to an amino acid is selected from the group consisting of a succimide group, an isothiocyanate group and an isocyanate group.

72. A method of comparing relative protein concentrations in a sample comprising
(a) providing a plurality of differential small molecule tags, wherein the differential small molecule tags comprise a chimeric labeling reagent comprising (i) a first domain comprising a biotin; and, (ii) a second domain comprising a reactive group capable of covalently binding to an amino acid, wherein the chimeric labeling reagent comprises at least one isotope;
(b) providing at least two samples comprising polypeptides;
(c) attaching covalently the differential small molecule tags to amino acids of the polypeptides;
(d) isolating the tagged polypeptides on a biotin-binding column by binding tagged polypeptides to the column, washing non-bound materials off the column, and eluting tagged polypeptides off the column;
(e) determining the protein concentrations of each sample in a tandem mass spectrometer; and,
(f) comparing relative protein concentrations of each sample.
